(19) United States
(12) Patent Application Publication
(10) Pub. No.: US 2021/0024483 A1
(43) Pub. Date: Jan. 28, 2021

(54) HETEROCYLCOALKENYL DERIVATIVES USEFUL AS AGONISTS OF THE GPR120 AND / OR GPR40

(71) Applicant: Janssen Pharmaceutica NV, Beerse (BE)

(72) Inventors: James C. Lanter, Cambridge, MA (US); Mark Wall, Lansdale, PA (US); Zhihua Sui, Norristown, PA (US)

(21) Appl. No.: 16/977,832

(22) PCT Filed: Mar. 5, 2019
(86) PCT No.: PCT/IB2019/051780

§ 371 Date: Sep. 3, 2020

Related U.S. Application Data
(60) Provisional application No. 62/639,029, filed on Mar. 6, 2018.

(51) Int. Cl. C07D 309/22; C07D 313/04

(52) U.S. Cl. CPC C07D 309/22; C07D 313/04

(57) ABSTRACT

The present invention is directed to heterocycloalkenyl derivatives, pharmaceutical compositions containing them and their use in the treatment of disorders and conditions modulated by the GPR120 and/or GPR40 receptors. More particularly, the compounds of the present invention are agonists of GPR120 and/or GPR40, useful in the treatment of, for example, obesity, Type II Diabetes Mellitus, dyslipidemia, etc.

### CROSS REFERENCE TO RELATED APPLICATIONS

This application claims priority to U.S. Provisional Patent Application No. 62/639,029, filed Mar. 6, 2018, the disclosure of which is hereby incorporated by reference in its entirety.

### FIELD OF THE INVENTION

The present invention is directed to heterocycloalkenyl derivatives, pharmaceutical compositions containing them and their use in the treatment of disorders and conditions modulated by the GPR120 and/or GPR40 receptors. More particularly, the compounds of the present invention are agonists of GPR120 and/or GPR40, useful in the treatment of, for example, obesity, Type II Diabetes Mellitus, dyslipidemia, etc.

### BACKGROUND OF THE INVENTION

A diabetes mellitus epidemic is unfolding across the globe with the World Health Organization (WHO) reporting a worldwide prevalence of 177 million patients with diabetes. It is estimated that the incidence of all forms of diabetes totals approximately 2.8% of the world population. The number of newly diagnosed diabetic patients is increasing by 4-5% per year. The total number of people with diabetes worldwide is projected to rise to 366 million (4.4% prevalence) in 2030. Type 2 diabetes accounts for approximately 95% of all diabetes cases. Long-term complications of type 2 diabetes include atherosclerosis, heart disease, stroke, end-stage renal disease, retinopathy leading to blindness, nerve damage, sexual dysfunction, frequent infections, and difficult-to-treat foot ulcers, sometimes resulting in lower limb amputation. Diabetics are twice as likely to develop cardiovascular disease or have a stroke, 2 to 6 times more likely to have transient ischemic attacks, and 15 to 40 times more likely to require lower-limb amputation compared with the general population. The total estimated cost of diabetes in 2007 in the US was $174 billion, including $116 billion in medical expenditures. The largest components of medical expenditures attributed to diabetes are hospital inpatient care (50% of total cost), diabetes medication and supplies (12%), retail prescriptions to treat complications of diabetes (11%), and physician office visits (9%). This may be related to the lack of durable efficacy of current drug therapies for Type 2 diabetes (>50% Type 2 patients are not reaching the targeted blood glucose control with current oral medications after 5 years of treatment). There is a general consensus that a considerable need exists for improved awareness, diagnosis and new, more effective, drug therapies for diabetes.

Agents that reduce hepatic glucose production, the so-called biguanides, such as metformin or phenformin, are generally preferred as the first-line of treatment for newly-diagnosed patients. Glitazones, such as rosiglitazone and pioglitazone function as insulin sensitizers (i.e., enhance insulin action) through the activation of peroxisome proliferator-activated receptor-gamma. (PPAR-gamma). These agents can provide the benefit of enhanced insulin action in tissues such as muscle, liver and adipose, but their use is frequently accompanied by increased weight and edema. In addition, rosiglitazone has recently been linked to heart attacks and its use has subsequently been more restricted. The insulin secretagogue sulfonylureas (such as tolbutamide, chlorpropamide, glipizide or glyburide) enhance insulin secretion from functional beta cells and are often combined with biguanide or glitazone therapy. However, because their effects on stimulating insulin release are independent of glucose levels, the sulfonylureas bear the risk of inducing incidences of hypoglycemia. Weight gain is also a common side-effect from this compound class.

Dipeptidyl-peptidase-4 (DPP-4) inhibitors (the so-called, “gliptins” such as sitagliptin, saxagliptin, linagliptin, vildagliptin, anagliptin or alogliptin) inhibit the metabolic degradation of endogenous incretins and thereby provide indirect increases in insulin secretion in response to elevations in circulating glucose levels.

GLP-1 is secreted from specific cells in the colon according to a meal and is a key regulator of glucose homeostasis, linking the gut, brain and pancreas. GLP-1 potentiates insulin secretion, reduces glucagon secretion and preserves β-cell function whilst also improving satiety. Levels of post-prandial GLP-1 are reduced in type 2 diabetics and dramatically elevated according to gastric by-pass surgery, contributing to the amelioration of type 2 diabetes in these patients. Approaches that prolong the half-life of GLP-1 (JANUVIA (Merck), GALVUS (Novartis)) or activate the GLP-1 receptor (BYETTA (Amylin)) have been recently approved for use in type 2 diabetes.

Hyperinsulinemia in patients with type 2 diabetes mellitus results from peripheral insulin resistance, coupled with inadequate pancreatic insulin secretion and elevated glucagon levels. There is a strong correlation between obesity and peripheral insulin resistance and hyperinsulinemia. Accumulation of free fatty acids in insulin responsive tissues other than fat (i.e. muscle and liver) results in tissue insulin resistance. Additionally, free fatty acids have a direct effect on the pancreas and in the colon and further stimulate glucose-dependent insulin secretion and GLP-1 release with acute exposure whereas chronic exposure of free fatty acids impairs insulin secretion and becomes toxic to the β-cell. In the liver, hyperinsulinemia per se has been linked to exacerbating insulin resistance by increasing liver fatty acid accumulation and hepatic glucose output creating a vicious cycle of disease progression. Current therapeutic strategies only partially address the complex pathology of free fatty acids in the exacerbation of diabetes. Agents that target both liver and pancreas function, directly or indirectly via GLP-1 release, either individually or in combination with current treatment, could significantly improve blood glucose control while maintaining β-cell function. Agents that potentiate GLP-1 release also have the ability to reduce weight, providing additional benefits.

A decade ago, the discovery of the G-protein coupled receptor GPR40 as a fatty acid receptor specifically expressed in beta cells and which stimulates glucose-dependent insulin secretion, sparked interest in the pharmaceutical industry as a potential therapeutic target to enhance insulin secretion in type 2 diabetes. The recognition of GPR40 as a receptor whose activation enhances glucose-dependent insulin secretion has led to the search for selective agonists for this putative therapeutic target. GPR40, also known as free fatty acid receptor 1 (FFR1), is one of a family of G-protein coupled receptors that, through receptor deorphanization studies, was shown to be endogenously activated by medium- to long-chain saturated and unsaturated fatty acids (˜C₁₂₋₂₀) (BRISCO, et al., J. Biol. Chem., 2003, pp 11303-11311, Vol. 278; ITOH, et al., Nature, 2003, pp 173-176, Vol. 422: KOTARSKY, et al., Biochem. Biophys. Res. Commun., 2003, pp 406-410, Vol. 301). In humans and rodents, although present in brain and enteroendocrine cells, its expression is particularly high in pancreatic beta cells and enteroendocrine cells in the gut. Operating primarily through GC signaling, GPR40 activation of the beta cell leads to an increase in intracellular calcium levels, which in the presence of glucose, ultimately results in augmented insulin secretion. In enteroendocrine cells, GPR40 activation by fatty acids leads to stimulation of incretin secretion (EDFALK, et al., Diabetes, 2008, pp 2280-2287, Vol. 57). Thus, in addition to directly promoting GSIS from islet beta cells, GPR40 activation in enteroendocrine cells provides an indirect means of stimulating GSIS through the actions of released incretins.

Because of the hyperglycemic dependency of GPR40-mediated effects on insulin secretion, activation (including selective activation) of this receptor provides a unique potential therapeutic mechanism by which to treat the diabetic state with minimal risk of hypoglycemic incidents. Given the relatively restricted tissue expression pattern of GPR40, selective GPR40 agonists may offer the additional advantage of providing an improved safety profile relative to the other therapeutic agents. Thus, GPR40 receptor agonists may provide a therapeutic benefit for the treatment of diabetes, particularly Type II Diabetes Mellitus, as well as related disorders and conditions such as obesity, impaired glucose tolerance, insulin resistance, metabolic syndrome (also known as Syndrome X), etc.

GPR120 is a seven transmembrane g-protein coupled receptor (GPCR) that is predominantly expressed in the intestine and adipose. GPR120 functions as a receptor for long chain free fatty acids (FFAs). Acute FFA stimulation of GPR120 in GLP-1 expressing cell-lines amplifies GLP-1 release. Administration of α-linolenic acid into the colon of mice increases GLP-1 and potentiates insulin release according to a glucose challenge. In contrast to agonists of GPR40, the existing literature suggests that a GPR120 agonist would potentiate insulin secretion and reduce glucagon indirectly via GLP-1 release. GPR120 is also expressed in adipose, with expression induced during differentiation. Inhibition of GPR120 expression in 3T3-L1 adipocytes has been shown to reduce adipocyte differentiation. The role of the receptor in the adipose or in the taste cells of the tongue where it has also been found remains unclear.

GPR120 is a Gq coupled GPCR that acts a receptor for long chain fatty acids. It belongs to a family of lipid binding GPCRs that include GPR 40, 41, 43. Functionally, GPR120s closest homolog is GPR40. The cloned rat and mouse GPR120 receptors have been cloned and have >85% homology with the human receptor. GPR120 signals through Gq to elevate intracellular Ca+2 levels as well as activate MAP kinase signal transduction cascades. GPR120's activation of calcium flux and PKC activation is most likely how FFAs contribute to the release GLP-1 in the L-cell.

Although relatively little is known about GPR120 due to lack of potent, selective pharmacological tools or a documented metabolic phenotype of GPR120 knockout mice, the potential to elevate GLP-1 from a small-molecule perspective is attractive as a novel approach to unmet medical need in the treatment of type 2 diabetes mellitus and related disorders. The beneficial effects of elevating GLP-1 are already well validated in the clinic and in addition to improved glucose homeostasis, offer the potential of weight loss. Thus it is theorized that GPR120 agonists may be complementary to existing diabetes therapies that affect liver insulin sensitivity and those that preserve β-cell function.

KUMAR, S., et al., in PCT Patent Publication WO2016/012965 A2, published Jan. 28, 2016 describe substituted phenyl alkanoic acid compounds as GPR120 agonists and uses thereof.

MALM, J., et al., in U.S. Pat. No. 7,005,538 B1, Issued Feb. 28, 2006 describe thyroid receptor ligands, preferably antagonists, useful for the treatment of cardiac arrhythmias, thyrotoxicosis and subclinical hyperthyroidism.

MOELLER, H. M., et al., in U.S. Pat. No. 4,939,171, issued Jul. 3, 1990 describe alkyl and alkenyl aryl ether derivatives useful as sebosuppressive agents in cosmetic or pharmaceutical preparations for topical application to the hair and to the skin.

There remains a need for GPR120, GPR40 and dual GPR120/GPR40 agonists for the treatment of disorders including, but not limited to obesity, Type II Diabetes Mellitus, dyslipidemia, and the like.

### SUMMARY OF THE INVENTION

The present invention is directed to compounds of formula (I)

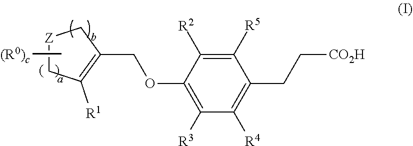

wherein

a is an integer from 1 to 2;

b is an integer from 1 to 2;

Z is selected from the group consisting of —O—, —S— and —SO₂—;

provided that when a is 1 and b is 2, or when a is 2 and b is 1 or when a is 2 and b is 2, then Z is —O—;

c is an integer from 0 to 4;

each R⁰ is independently selected from the group consisting of halogen, oxo, hydroxy, cyano, C₁₋₄alkyl, fluorinated C₁₋₂alkyl, C₁₋₄alkoxy and fluorinated C₁₋₂alkoxy;

R¹ is selected from the group consisting of C₁₋₆alkyl, C₂₋₆alkynyl, C₃₋₆cycloalkyl, —(C₁₋₂alkyl)-C₃₋₆cycloalkyl, C₅₋₆cycloalkenyl bicyclo[3.1.0]hexy-2-yl, (1S,4S)-2-methyl-bicyclo[2.2.1]hept-2-yl, (1R,2R,4S)-bicyclo[2.2.1]heptan-2-yl, 2,2-difluoro-benzo[d][1,3]dioxol-4-yl, phenyl, —(C₁₋₂alkyl)-phenyl, —C(═CH)-phenyl, —C(O)-phenyl, tetrahydropyranyl, furanyl, pyrimidinyl, pyridyl, thienyl, thiazolyl, —(C₁₋₂alkyl)-thiazolyl, 3,6-dihydro-pyran-4-yl and 1-methyl-imidazol-4-yl;

wherein the C₃₋₆cycloalkyl or C₅₋₆cycloalkenyl, whether alone or as part of a substituent group is optionally substituted with one or more (preferably one to two) substituents independently selected from the group consisting of halogen, C₁₋₄alkyl and fluorinated C₁₋₂alkyl;

and wherein the phenyl, furanyl, pyrimidinyl, pyridyl, thienyl, thiazolyl, or 3,6-dihydro-pyran-4-yl, whether alone or as part of a substituent group is optionally substituted with one or more (preferably one to three) substituents independently selected from the group consisting of halogen, hydroxy, cyano, C₁₋₄alkyl, fluorinated C₁₋₂alkyl, C₁₋₄alkoxy, fluorinated C₁₋₂alkoxy, —S(C₁₋₂alkyl), —SO—(C₁₋₂alkyl), —SO₂—(C₁₋₂alkyl), nitro, —NR^{A}R^{B}, —NH—C(O)—(C₁₋₄alkyl) and phenyl; wherein R^{A} and R^{B} are each independently selected from the group consisting of hydrogen and C₁₋₄alkyl;

R² is selected from the group consisting of hydrogen, halogen, C₁₋₄alkyl, fluorinated C₁₋₂alkyl, —(C₁₋₄alkyl)-S—(C₁₋₂alkyl), —(C₁₋₄alkyl)-SO—(C₁₋₂alkyl) and —(C₁₋₄alkyl)-SO₂—(C₁₋₂alkyl);

R³ is selected from the group consisting of hydrogen, halogen, C₁₋₄alkyl and fluorinated C₁₋₂alkyl;

R⁴ is selected from the group consisting of hydrogen, halogen, C₁₋₄alkyl and fluorinated C₁₋₂alkyl;

R⁵ is selected from the group consisting of hydrogen, cyano, halogen, C₁₋₄alkyl, fluorinated C₁₋₂alkyl, vinyl, halogen substituted vinyl, ethynyl, hydroxy substituted C₁₋₂alkyl, C₁₋₄alkoxy, fluorinated C₁₋₂alkoxy, cyclopropyl, cyclopropyl-methyl- and phenyl;

alternatively, R² and R⁵ or R³ and R⁴ are taken together with the carbon atoms to which they are bound to form cyclopenten-1-yl;

and pharmaceutically acceptable salts thereof.

The present invention is further directed to processes for the preparation of the compounds of formula (I). The present invention is further directed to a compound prepared according to any of the process(es) described herein.

Illustrative of the invention is a pharmaceutical composition comprising a pharmaceutically acceptable carrier and the compound prepared according to the process described herein. An illustration of the invention is a pharmaceutical composition made by mixing the compound prepared according to the process described herein and a pharmaceutically acceptable carrier. Illustrating the invention is a process for making a pharmaceutical composition comprising mixing the compound prepared according to the process described herein and a pharmaceutically acceptable carrier.

Exemplifying the invention are methods of treating a disorder modulated by GPR120 such as obesity, obesity induced inflammation, impaired glucose tolerance, elevated fasting glucose, insulin resistance, hyperglycemia, hyperinsulinemia, Type II Diabetes Mellitus, metabolic syndrome (also known as Syndrome X), gestational diabetes, diabetic retinopathy, kidney disease, ketoacidosis, diabetic nephropathy, dyslipidemia, elevated LDL, hyperipidemia, hyperlipoproteinemia, hypertriglyceridemia (i.e. elevated triglycerides), non-alcoholic steatohepatitis (NASH), non-alcoholic fatty liver disease (NAFLD) or liver fibrosis, comprising administering to a subject in need thereof a therapeutically effective amount of any of the compounds or pharmaceutical compositions described above.

In an embodiment, the present invention is directed to a compound of formula (I) for use as a medicament. In another embodiment, the present invention is directed to a compound of formula (I) for use in the treatment of a disorder modulated by GPR120 such as obesity, obesity induced inflammation, impaired glucose tolerance, elevated fasting glucose, insulin resistance, hyperglycemia, hyperinsulinemia, Type II Diabetes Mellitus, metabolic syndrome (also known as Syndrome X), gestational diabetes, diabetic retinopathy, kidney disease, ketoacidosis, diabetic nephropathy, dyslipidemia, elevated LDL, hyperipidemia, hyperipoproteinemia, hypertriglyceridemia (i.e. elevated triglycerides), non-alcoholic steatohepatitis (NASH), non-alcoholic fatty liver disease (NAFLD) or liver fibrosis.

In another embodiment, the present invention is directed to a composition comprising a compound of formula (I) for the treatment of a disorder modulated by GPR120 such as obesity, obesity induced inflammation, impaired glucose tolerance, elevated fasting glucose, insulin resistance, hyperglycemia, hyperinsulinemia, Type II Diabetes Mellitus, metabolic syndrome (also known as Syndrome X), gestational diabetes, diabetic retinopathy, kidney disease, ketoacidosis, diabetic nephropathy, dyslipidemia, elevated LDL, hyperipidemia, hyperlipoproteinemia, hypertriglyceridemia (i.e. elevated triglycerides), non-alcoholic steatohepatitis (NASH), non-alcoholic fatty liver disease (NAFLD) or liver fibrosis.

Another example of the invention is the use of any of the compounds described herein in the preparation of a medicament for treating: (a) obesity, (b) obesity induced inflammation, (c) impaired glucose tolerance, (d) elevated fasting glucose, (e) insulin resistance, (f) hyperglycemia, (g) hyperinsulinemia, (h) Type II Diabetes Mellitus, (j) metabolic syndrome (also known as Syndrome X), (j) gestational diabetes, (k) diabetic retinopathy, (l) kidney disease, (m) ketoacidosis, (n) diabetic nephropathy, (o) dyslipidemia, (p) elevated LDL, (q) hyperlipidemia, (r) hyperlipoproteinemia, (s) hypertriglyceridemia (i.e. elevated triglycerides), (t) non-alcoholic steatohepatitis (NASH), (u) non-alcoholic fatty liver disease (NAFLD) and (v) liver fibrosis, in a subject in need thereof.

In another example, the present invention is directed to a compound as described herein for use in a methods for treating a disorder selected from the group consisting of obesity, obesity induced inflammation, impaired glucose tolerance, elevated fasting glucose, insulin resistance, hyperglycemia, hyperinsulinemia, Type II Diabetes Mellitus, metabolic syndrome (also known as Syndrome X), gestational diabetes, diabetic retinopathy, kidney disease, ketoacidosis, diabetic nephropathy, dyslipidemia, elevated LDL, hyperipidemia, hyperlipoproteinemia, hypertriglyceridemia (i.e. elevated triglycerides), non-alcoholic steatohepatitis (NASH), non-alcoholic fatty liver disease (NAFLD) and liver fibrosis, in a subject in need thereof.

Exemplifying the invention are methods of treating a disorder modulated by GPR40 such as obesity, obesity induced inflammation, impaired glucose tolerance, elevated fasting glucose, insulin resistance, hyperglycemia, hyperinsulinemia, Type II Diabetes Mellitus, metabolic syndrome (also known as Syndrome X), gestational diabetes, diabetic retinopathy, kidney disease, ketoacidosis, diabetic nephropathy, dyslipidemia, elevated LDL, hyperlipidemia, hyperlipoproteinemia, hypertriglyceridemia (i.e. elevated triglycerides), non-alcoholic steatohepatitis (NASH), non-alcoholic fatty liver disease (NAFLD), liver fibrosis or cardiovascular disorders (including but not limited to hypertension, atherosclerosis, thrombotic disorders, and cardiac fibrosis), comprising administering to a subject in need thereof a therapeutically effective amount of any of the compounds or pharmaceutical compositions described above.

In an embodiment, the present invention is directed to a compound of formula (I) for use as a medicament. In another embodiment, the present invention is directed to a compound of formula (I) for use in the treatment of a disorder modulated by GPR40 such as obesity, obesity induced inflammation, impaired glucose tolerance, elevated fasting glucose, insulin resistance, hyperglycemia, hyperinsulinemia, Type II Diabetes Mellitus, metabolic syndrome (also known as Syndrome X), gestational diabetes, diabetic retinopathy, kidney disease, ketoacidosis, diabetic nephropathy, dyslipidemia, elevated LDL, hyperlipidemia, hyperlipoproteinemia, hypertriglyceridemia (i.e. elevated triglycerides), non-alcoholic steatohepatitis (NASH), non-alcoholic fatty liver disease (NAFLD), liver fibrosis or cardiovascular disorders (including but not limited to hypertension, atherosclerosis, thrombotic disorders, and cardiac fibrosis).

In another embodiment, the present invention is directed to a composition comprising a compound of formula (I) for the treatment of a disorder modulated by GPR40 such as obesity, obesity induced inflammation, impaired glucose tolerance, elevated fasting glucose, insulin resistance, hyperglycemia, hyperinsulinemia, Type II Diabetes Mellitus, metabolic syndrome (also known as Syndrome X), gestational diabetes, diabetic retinopathy, kidney disease, ketoacidosis, diabetic nephropathy, dyslipidemia, elevated LDL, hyperlipidemia, hyperlipoproteinemia, hypertriglyceridemia (i.e. elevated triglycerides), non-alcoholic steatohepatitis (NASH), non-alcoholic fatty liver disease (NAFLD), liver fibrosis or cardiovascular disorders (including but not limited to hypertension, atherosclerosis, thrombotic disorders, and cardiac fibrosis).

Another example of the invention is the use of any of the compounds described herein in the preparation of a medicament for treating: (a) obesity, (b) obesity induced inflammation, (c) impaired glucose tolerance, (d) elevated fasting glucose, (e) insulin resistance, (f) hyperglycemia, (g) hyperinsulinemia, (h) Type II Diabetes Mellitus, (i) metabolic syndrome (also known as Syndrome X), (j) gestational diabetes, (k) diabetic retinopathy, (l) kidney disease, (m) ketoacidosis, (n) diabetic nephropathy, (o) dyslipidemia, (p) elevated LDL, (q) hyperlipidemia, (r) hyperlipoproteinemia, (s) hypertriglyceridemia (i.e. elevated triglycerides), (t) non-alcoholic steatohepatitis (NASH), (u) non-alcoholic fatty liver disease (NAFLD), (v) liver fibrosis and (w) cardiovascular disorders (including but not limited to hypertension, atherosclerosis, thrombotic disorders, and cardiac fibrosis), in a subject in need thereof.

In another example, the present invention is directed to a compound as described herein for use in a methods for treating a disorder selected from the group consisting of obesity, obesity induced inflammation, impaired glucose tolerance, elevated fasting glucose, insulin resistance, hyperglycemia, hyperinsulinemia, Type II Diabetes Mellitus, metabolic syndrome (also known as Syndrome X), gestational diabetes, diabetic retinopathy, kidney disease, ketoacidosis, diabetic nephropathy, dyslipidemia, elevated LDL, hyperipidemia, hyperlipoproteinemia, hypertriglyceridemia (i.e. elevated triglycerides), non-alcoholic steatohepatitis (NASH), non-alcoholic fatty liver disease (NAFLD), liver fibrosis and cardiovascular disorders (including but not limited to hypertension, atherosclerosis, thrombotic disorders, and cardiac fibrosis), in a subject in need thereof.

Exemplifying the invention are methods of treating a disorder which responds to dual agonism of the GPR120 and GP40 receptors such as obesity, obesity induced inflammation, impaired glucose tolerance, elevated fasting glucose, insulin resistance, hyperglycemia, hyperinsulinemia, Type II Diabetes Mellitus, metabolic syndrome (also known as Syndrome X), gestational diabetes, diabetic retinopathy, kidney disease, ketoacidosis, diabetic nephropathy, dyslipidemia, elevated LDL, hyperipidemia, hyperipoproteinemia, hypertriglyceridemia (i.e. elevated triglycerides), non-alcoholic steatohepatitis (NASH), non-alcoholic fatty liver disease (NAFLD), liver fibrosis or cardiovascular disorders (including but not limited to hypertension, atherosclerosis, thrombotic disorders, and cardiac fibrosis), comprising administering to a subject in need thereof a therapeutically effective amount of any of the compounds or pharmaceutical compositions described above.

In an embodiment, the present invention is directed to a compound of formula (I) for use as a medicament. In another embodiment, the present invention is directed to a compound of formula (I) for use in the treatment of a disorder which responds to dual agonism of the GPR120 and GP40 receptors such as obesity, obesity induced inflammation, impaired glucose tolerance, elevated fasting glucose, insulin resistance, hyperglycemia, hyperinsulinemia, Type II Diabetes Mellitus, metabolic syndrome (also known as Syndrome X), gestational diabetes, diabetic retinopathy, kidney disease, ketoacidosis, diabetic nephropathy, dyslipidemia, elevated LDL, hyperlipidemia, hyperlipoproteinemia, hypertriglyceridemia (i.e. elevated triglycerides), non-alcoholic steatohepatitis (NASH), non-alcoholic fatty liver disease (NAFLD), liver fibrosis or cardiovascular disorders (including but not limited to hypertension, atherosclerosis, thrombotic disorders, and cardiac fibrosis).

In another embodiment, the present invention is directed to a composition comprising a compound of formula (I) for the treatment of a disorder which responds to dual agonism of the GPR120 and GP40 receptors such as obesity, obesity induced inflammation, impaired glucose tolerance, elevated fasting glucose, insulin resistance, hyperglycemia, hyperinsulinemia, Type II Diabetes Mellitus, metabolic syndrome (also known as Syndrome X), gestational diabetes, diabetic retinopathy, kidney disease, ketoacidosis, diabetic nephropathy, dyslipidemia, elevated LDL, hyperlipidemia, hyperlipoproteinemia, hypertriglyceridemia (i.e. elevated triglycerides), non-alcoholic steatohepatitis (NASH), non-alcoholic fatty liver disease (NAFLD), liver fibrosis or cardiovascular disorders (including but not limited to hypertension, atherosclerosis, thrombotic disorders, and cardiac fibrosis).

Another example of the invention is the use of any of the compounds described herein in the preparation of a medicament for treating: (a) obesity, (b) obesity induced inflammation, (c) impaired glucose tolerance, (d) elevated fasting glucose, (e) insulin resistance, (f) hyperglycemia, (g) hyperinsulinemia, (h) Type II Diabetes Mellitus, (i) metabolic syndrome (also known as Syndrome X), (j) gestational diabetes, (k) diabetic retinopathy, (l) kidney disease, (m) ketoacidosis, (n) diabetic nephropathy, (o) dyslipidemia, (p) elevated LDL, (q) hyperlipidemia, (r) hyperlipoproteinemia, (s) hypertriglyceridemia (i.e. elevated triglycerides), (u) non-alcoholic steatohepatitis (NASH), (u) non-alcoholic fatty liver disease (NAFLD), (v) liver fibrosis and (w) cardiovascular disorders (including but not limited to hypertension, atherosclerosis, thrombotic disorders, and cardiac fibrosis), in a subject in need thereof.

In another example, the present invention is directed to a compound as described herein for use in a methods for treating a disorder selected from the group consisting of obesity, obesity induced inflammation, impaired glucose tolerance, elevated fasting glucose, insulin resistance, hyperglycemia, hyperinsulinemia, Type II Diabetes Mellitus, metabolic syndrome (also known as Syndrome X), gestational diabetes, diabetic retinopathy, kidney disease, ketoacidosis, diabetic nephropathy, dyslipidemia, elevated LDL, hyperipidemia, hyperlipoproteinemia, hypertriglyceridemia (i.e. elevated triglycerides), non-alcoholic steatohepatitis (NASH), non-alcoholic fatty liver disease (NAFLD), liver fibrosis and cardiovascular disorders (including but not limited to hypertension, atherosclerosis, thrombotic disorders, and cardiac fibrosis), in a subject in need thereof.

### DETAILED DESCRIPTION OF THE INVENTION

The present invention is directed to compounds of formula (I)

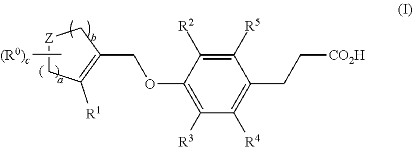

wherein a, b, Z, c, R⁰, R¹, R², R³, R⁴ and R⁵ are as herein defined. The compounds of the present invention are agonists of the GPR120 and/or GPR40 receptors, useful in the treatment of disorders and diseases which are modulated by said receptors, including for example, obesity, obesity induced inflammation, impaired glucose tolerance, elevated fasting glucose, insulin resistance, hyperglycemia, hyperinsulinemia, Type II Diabetes Mellitus, metabolic syndrome (also known as Syndrome X), gestational diabetes, diabetic retinopathy, kidney disease, ketoacidosis, diabetic nephropathy, dyslipidemia, elevated LDL, hyperipidemia, hyperipoproteinemia, hypertriglyceridemia (i.e. elevated triglycerides), non-alcoholic steatohepatitis (NASH), non-alcoholic fatty liver disease (NAFLD), liver fibrosis and cardiovascular disorders (including but not limited to hypertension, atherosclerosis, thrombotic disorders, and cardiac fibrosis). Preferably, the disorder or disease is selected from the group consisting of obesity, impaired glucose tolerance, elevated fasting glucose, insulin resistance, hyperglycemia, hyperinsulinemia, Type II Diabetes Mellitus, metabolic syndrome (also known as Syndrome X), diabetic retinopathy, diabetic nephropathy, dyslipidemia, elevated LDL, hyperlipidemia, hypertriglyceridemia (i.e. elevated triglycerides), non-alcoholic steatohepatitis (NASH) and non-alcoholic fatty liver disease (NAFLD). More preferably, the disorder or disease is selected from the group consisting of obesity, Type II diabetes, metabolic syndrome (also known as Syndrome X), dyslipidemia. hypertriglyceridemia (i.e. elevated triglycerides), non-alcoholic steatohepatitis (NASH) and non-alcoholic fatty liver disease (NAFLD). More preferably, the disorder or disease is selected from the group consisting of obesity, Type II diabetes, metabolic syndrome (also known as Syndrome X), dyslipidemia and hypertriglyceridemia.

In an embodiment, the present invention is directed to compounds of formula (A)

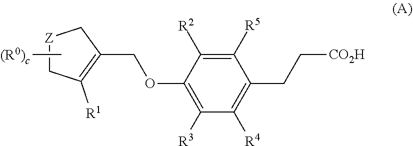

(compounds of formula (I) wherein a is 1 and b is 1 and wherein Z is selected from the group consisting of —O—, —S— and —SO₂—), and pharmaceutically acceptable salts thereof.

In another embodiment, the present invention is directed to compounds of formula (B)

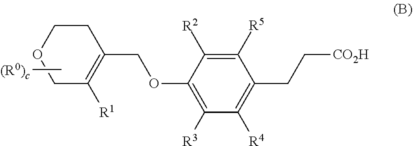

(compounds of formula (I) wherein a is 1 and b is 2), and pharmaceutically acceptable salts thereof.

In another embodiment, the present invention is directed to compounds of formula (C)

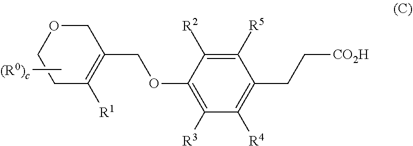

(compounds of formula (I) wherein a is 2 and b is 1), and pharmaceutically acceptable salts thereof.

In another embodiment, the present invention is directed to compounds of formula (D)

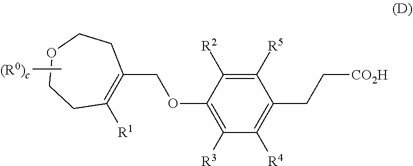

(compounds of formula (I) wherein a is 2 and b is 2), and pharmaceutically acceptable salts thereof.

In certain embodiments, the present invention is directed to compounds of formula (I) wherein a is 1 and b is 1. In certain embodiments, the present invention is directed to compounds of formula (I) wherein a is 2 and b is 1. In certain embodiments, the present invention is directed to compounds of formula (I) wherein a is 1 and b is 2. In certain embodiments, the present invention is directed to compounds of formula (I) wherein a is 2 and b is 2.

In certain embodiments, the present invention is directed to compounds of formula (I) wherein a is an integer from 1 to 2; and b is an integer from 1 to 2. In certain embodiments, the present invention is directed to compounds of formula (I) wherein a is an integer from 1 to 2 and b is 2; or wherein a is 2 and b is an integer from 1 to 2. In certain embodiments, the present invention is directed to compounds of formula (I) wherein a is 1 and b is 2; or wherein a is 2 and b is 1. In certain embodiments, the present invention is directed to compounds of formula (I) wherein a is 1 and b is 1; or a is 2 and b is 1; or a is 2 and b is 2.

In certain embodiments, the present invention is directed to compounds of formula (I) wherein Z is selected from the group consisting of —O—, —S—, —SO— and —SO₂—; provided that when a is 1 and b is 2, or when a is 2 and b is 1 or when a is 2 and b is 2, then Z is —O—. In certain embodiments, the present invention is directed to compounds of formula (I) wherein Z is selected from the group consisting of —O— and —S—. In certain embodiments, the present invention is directed to compounds of formula (I) wherein Z is —O—.

In certain embodiments, the present invention is directed to compounds of formula (I) wherein c is an integer from 0 to 2. In certain embodiments, the present invention is directed to compounds of formula (I) wherein c is an integer from 0 to 1. In certain embodiments, the present invention is directed to compounds of formula (I) wherein c is 0. In certain embodiments, the present invention is directed to compounds of formula (I) wherein c is 2. In certain embodiments, the present invention is directed to compounds of formula (I) wherein c is 2.

In certain embodiments, the present invention is directed to compounds of formula (I) wherein each R⁰ is independently selected from the group consisting of halogen, oxo, hydroxy, C₁₋₄alkyl, fluorinated C₁₋₂alkyl, C₁₋₂alkoxy and fluorinated C₁₋₂alkoxy. In certain embodiments, the present invention is directed to compounds of formula (I) wherein each R⁰ is independently selected from the group consisting of C₁₋₄alkyl. In certain embodiments, the present invention is directed to compounds of formula (I) wherein each R⁰ is methyl. In certain embodiments, the present invention is directed to compounds of formula (I) wherein each R⁰ is 6-methyl.

In certain embodiments, the present invention is directed to compounds of formula (I) wherein R¹ is selected from the group consisting of C₁₋₆alkyl, C₃₋₆cycloalkyl, —(C₁₋₂alkyl)-C₃₋₆cycloalkyl, C₅₋₆cycloalkenyl, 2,2-difluoro-benzo[d][1,3]dioxol-4-yl, phenyl, —(C₁₋₂alkyl)-phenyl, —C(═CH)-phenyl, —C(O)-phenyl, pyrimidinyl, pyridyl, thienyl, and 1-methyl-imidazol-4-yl; wherein the C₃₋₆cycloalkyl or C₅₋₆cycloalkenyl, whether alone or as part of a substituent group is optionally substituted with one to two substituents independently selected from the group consisting of halogen, C₁₋₂alkyl and fluorinated C₁₋₂alkyl; and wherein the phenyl, pyrimidinyl or pyridyl, whether alone or as part of a substituent group is optionally substituted with one to three substituents independently selected from the group consisting of halogen, hydroxy, C₁₋₄ alkyl, fluorinated C₁₋₂alkyl, C₁₋₄alkoxy, fluorinated C₁₋₂alkoxy, and phenyl;

In certain embodiments, the present invention is directed to compounds of formula (I) wherein R¹ is selected from the group consisting of C₁₋₆alkyl, —C(═CH)-phenyl, C₃₋₆cycloalkyl, C₅₋₆cycloalkenyl, —(C₁₋₂alkyl)-C₅₋₆cycloalkyl, phenyl, —(C₁₋₂alkyl)-phenyl, pyrid-3-yl, and 2,2-difluoro-benzo[d][1,3]dioxol-4-yl; wherein the C₅₋₆cycloalkylenyl, is optionally substituted with one to two C₁₋₂alkyl; wherein the phenyl, whether alone or as part of a substituent group, is optionally substituted with one to three substituents independently selected from the group consisting of halogen, C₁₋₄alkyl, fluorinated C₁₋₂alkyl, C₁₋₄alkoxy, fluorinated C₁₋₂alkoxy and phenyl.

In certain embodiments, the present invention is directed to compounds of formula (I) wherein R¹ is selected from the group consisting of ethyl, isopropyl, n-butyl, 1-methyl-n-propyl, isobutyl, isopentyl, n-hexyl, cyclobutyl, cyclopentyl, cyclohexyl, cyclopent-2-en-1-yl, 5,5-dimethyl-cyclopenten-1-yl, cyclohexyl-methyl-, phenyl, 2-fluorophenyl, 4-fluorophennyl, 4-chlorophenyl, 4-methylphenyl, 2-methoxyphenyl, 3-methoxyphenyl, 4-methoxyphenyl, 2-isopropyloxyphenyl, 2-trifluoromethoxy-phenyl, 4-trifluoromethoxy-phenyl, 2,4-difluorophenyl, 3,4-difluorophenyl, 3,4-dichlorophenyl, 2-fluoro-4-chloro-phenyl, 2-fluoro-4-methyl-phenyl, 3-fluoro-4-chloro-phenyl, 3-fluoro-4-methyl-phenyl, 2-methyl-4-chloro-phenyl, 3-methyl-4-fluoro-phenyl, 3-methyl-4-chloro-phenyl, 2-isopropyl-4-methyl-phenyl, 3-isopropyl-4-methyl-phenyl, 2-methoxy-4-chloro-phenyl, 3-methoxy-4-chloro-phenyl, 2,4-dimethyl-phenyl, 2,6-dimethyl-phenyl, 3,4-dimethyl-phenyl, 2,4,5-trimethyl-phenyl, 2-phenyl-4-methyl-phenyl, 3-phenyl-4-methyl-phenyl, benzyl, 4-chloro-benzyl, 4-methyl-benzyl, 4-methoxy-benzyl, 1-phenyl-ethyl-, 1-phenyl-vinyl-, 1-(4-fluorophenyl)-vinyl-, 1-(2-chloro-phenyl)-vinyl-, 1-(4-trifluoromethyl-phenyl)-vinyl-, pyrid-3-yl, and 2,2-difluoro-benzo[d][1,3]dioxol-4-yl.

In certain embodiments, the present invention is directed to compounds of formula (I) wherein R¹ is selected from the group consisting of phenyl, 4-chlorophenyl, 4-fluorophenyl and cyclopentyl.

In certain embodiments, the present invention is directed to compounds of formula (I) wherein R¹ is selected from the group consisting of ethyl, isopropyl, n-butyl, 1-methyl-n-propyl, isobutyl, isopentyl, n-hexyl, 2-fluorophenyl, 4-fluorophennyl, 4-chlorophenyl, 4-methylphenyl, 2-methoxyphenyl, 3-methoxyphenyl, 4-methoxyphenyl, 2-isopropyloxyphenyl, 2-trifluoromethoxy-phenyl, 4-trifluoromethoxy-phenyl, 2,4-difluorophenyl, 3,4-difluorophenyl, 3,4-dichlforophenyl, 2-fluoro-4-chloro-phenyl, 2-fluoro-4-methyl-phenyl, 3-fluoro-4-chloro-phenyl, 3-fluoro-4-methyl-phenyl, 2-methyl-4-chloro-phenyl, 3-methyl-4-fluoro-phenyl, 3-methyl-4-chloro-phenyl, 2-methoxy-4-chloro-phenyl, 3-methoxy-4-chloro-phenyl, 2,6-dimethyl-phenyl, 3,4-dimethyl-phenyl, 2,4,5-trimethyl-phenyl, benzyl, 4-chloro-benzyl, 4-methyl-benzyl, 4-methoxy-benzyl, 1-phenyl-ethyl-, 1-phenyl-vinyl-, 1-(4-fluorophenyl)-vinyl-, 1-(2-chloro-phenyl)-vinyl-, 1-(4-trifluoromethyl-phenyl)-vinyl-, cyclobutyl, cyclopentyl, cyclohexyl, cyclopent-2-en-1-yl, 5,5-dimethyl-cyclopenten-1-yl, cyclohexyl-methyl-, pyrid-3-yl, and 2,2-difluoro-benzo[d][1,3]dioxol-4-yl.

In certain embodiments, the present invention is directed to compounds of formula (I) wherein R¹ is selected from the group consisting of phenyl, 4-chlorophenyl, 4-methylphenyl, 2-fluoro-4-methyl-phenyl, 3-fluoro-4-methyl-phenyl, 2,4-dimethyl-phenyl, 2-isopropyl-4-methyl-phenyl, 3-isopropyl-4-methyl-phenyl, 2-phenyl-4-methyl-phenyl, 3-phenyl-4-methyl-phenyl, and cyclopentyl.

In certain embodiments, the present invention is directed to compounds of formula (I) wherein R¹ is selected from the group consisting of 4-chlorophenyl, 4-methylphenyl, 3-fluoro-4-methyl-phenyl, and cyclopentyl.

In certain embodiments, the present invention is directed to compounds of formula (I) wherein R¹ is selected from the group consisting of ethyl, isopropyl, 1-methyl-n-propyl, n-butyl, isobutyl, isopentyl, cyclobutyl, cyclopentyl, cyclohexyl, cyclopent-2-en-1-yl, 4-trifluoromethoxy-phenyl, 3-fluoro-4-methyl-phenyl, benzyl, 4-chloro-benzyl, 4-methyl-benzyl, 4-methoxy-benzyl, 1-phenyl-vinyl, 1-(2-chloro-phenyl)-vinyl, 1-(4-fluoro-phenyl)-vinyl and 1-(4-trifluoromethyl-phenyl)-vinyl. In certain embodiments, the present invention is directed to compounds of formula (I) wherein R¹ is selected from the group consisting of isopropyl, n-butyl, isobutyl, cyclopentyl, cyclopent-2-en-1-yl, 4-chloro-benzyl, 4-methyl-benzyl, 4-methoxy-benzyl, 1-phenyl-vinyl, 1-(2-chloro-phenyl)-vinyl, 1-(4-fluoro-phenyl)-vinyl and 1-(4-trifluoromethyl-phenyl)-vinyl. In certain embodiments, the present invention is directed to compounds of formula (I) wherein R¹ is selected from the group consisting of isobutyl, cyclopentyl, cyclopent-2-en-1-yl, 4-chloro-benzyl, 1-phenyl-vinyl, 1-(2-chloro-phenyl)-vinyl, 1-(4-fluoro-phenyl)-vinyl and 1-(4-trifluoromethyl-phenyl)-vinyl. In certain embodiments, the present invention is directed to compounds of formula (I) wherein R¹ is selected from the group consisting of cyclopentyl, 1-phenyl-vinyl, 1-(2-chloro-phenyl)-vinyl, 1-(4-fluoro-phenyl)-vinyl and 1-(4-trifluoromethyl-phenyl)-vinyl.

In certain embodiments, the present invention is directed to compounds of formula (I) wherein R¹ is selected from the group consisting of cyclobutyl, cyclopentyl, phenyl, 4-chlorophenyl, 2-fluorophenyl, 4-fluorophenyl, 4-methylphenyl, 2,4-difluorophenyl, 2-fluoro-4-chloro-phenyl, 3,4-difluorophenyl, 3-fluoro-4-chloro-phenyl and 3-fluoro-4-methyl-phenyl. In certain embodiments, the present invention is directed to compounds of formula (I) wherein R¹ is selected from the group consisting of phenyl, 4-chlorophenyl, 2-fluorophenyl, 4-fluorophenyl, 4-methylphenyl, 2,4-difluorophenyl, 2-fluoro-4-chloro-phenyl, 3,4-difluorophenyl, 3-fluoro-4-chloro-phenyl and 3-fluoro-4-methyl-phenyl. In certain embodiments, the present invention is directed to compounds of formula (I) wherein R¹ is selected from the group consisting of 4-chlorophenyl, 4-methylphenyl and 3-fluoro-4-methyl-phenyl.

In certain embodiments, the present invention is directed to compounds of formula (I) wherein R² is selected from the group consisting of hydrogen, halogen, C₁₋₄alkyl and fluorinated C₁₋₂alkyl. In certain embodiments, the present invention is directed to compounds of formula (I) wherein R² is selected from the group consisting of hydrogen, halogen and C₁₋₄alkyl.

In certain embodiments, the present invention is directed to compounds of formula (I) wherein R² is selected from the group consisting of hydrogen, fluoro and methyl. In certain embodiments, the present invention is directed to compounds of formula (I) wherein R² is selected from the group consisting of fluoro and methyl. In certain embodiments, the present invention is directed to compounds of formula (I) wherein R² is hydrogen. In certain embodiments, the present invention is directed to compounds of formula (I) wherein R² is methyl.

In certain embodiments, the present invention is directed to compounds of formula (I) wherein R³ is selected from the group consisting of hydrogen, halogen, C₁₋₂alkyl and fluorinated C₁₋₂alkyl. In certain embodiments, the present invention is directed to compounds of formula (I) wherein R³ is selected from the group consisting of hydrogen and halogen. In certain embodiments, the present invention is directed to compounds of formula (I) wherein R³ is selected from the group consisting of hydrogen and fluoro. In certain embodiments, the present invention is directed to compounds of formula (I) wherein R³ is hydrogen.

In certain embodiments, the present invention is directed to compounds of formula (I) wherein R⁴ is selected from the group consisting of hydrogen, halogen, C₁₋₄alkyl and fluorinated C₁₋₂alkyl. In certain embodiments, the present invention is directed to compounds of formula (I) wherein R⁴ is selected from the group consisting of hydrogen, C₁₋₄alkyl and fluorinated C₁₋₂alkyl. In certain embodiments, the present invention is directed to compounds of formula (I) wherein R⁴ is selected from the group consisting of hydrogen, methyl and trifluoromethyl. In certain embodiments, the present invention is directed to compounds of formula (I) wherein R⁴ is selected from the group consisting of hydrogen and trifluoromethyl. In certain embodiments, the present invention is directed to compounds of formula (I) wherein R⁴ is selected from the group consisting of hydrogen and methyl. In certain embodiments, the present invention is directed to compounds of formula (I) wherein R⁴ is hydrogen.

In certain embodiments, the present invention is directed to compounds of formula (I) wherein R⁵ is selected from the group consisting of hydrogen, halogen, C₁₋₄alkyl, fluorinated C₁₋₂alkyl, hydroxy substituted C₁₋₂alkyl, C₁₋₄alkoxy, fluorinated C₁₋₂alkoxy and cyclopropyl-methyl-. In certain embodiments, the present invention is directed to compounds of formula (I) wherein R⁵ is selected from the group consisting of hydrogen, halogen, C₁₋₄alkyl, fluorinated C₁₋₂alkyl and cyclopropyl-methyl-.

In certain embodiments, the present invention is directed to compounds of formula (I) wherein R⁵ is selected from the group consisting of hydrogen, fluoro, chloro, methyl, difluoromethyl, trifluoromethyl and cyclopropyl-methyl-. In certain embodiments, the present invention is directed to compounds of formula (I) wherein R⁵ is selected from the group consisting of hydrogen, chloro, methyl, difluoromethyl, and trifluoromethyl. In certain embodiments, the present invention is directed to compounds of formula (I) wherein R⁵ is selected from the group consisting of hydrogen, fluoro, methyl and trifluoromethyl. In certain embodiments, the present invention is directed to compounds of formula (I) wherein R⁵ is selected from the group consisting of hydrogen, fluoro, methyl, trifluoromethyl, and cyclopropyl-methyl-. In certain embodiments, the present invention is directed to compounds of formula (I) wherein R⁵ is selected from the group consisting of hydrogen, fluoro, chloro, methyl and trifluoromethyl. In certain embodiments, the present invention is directed to compounds of formula (I) wherein R⁵ is selected from the group consisting of hydrogen, fluoro and methyl. In certain embodiments, the present invention is directed to compounds of formula (I) wherein R⁵ is selected from the group consisting of chloro and trifluoromethyl. In certain embodiments, the present invention is directed to compounds of formula (I) wherein R⁵ is trifluoromethyl. In certain embodiments, the present invention is directed to compounds of formula (I) wherein R⁵ is methyl.

In certain embodiments, the present invention is directed to compounds of formula (I) wherein R² and R⁵ or R³ and R⁴ are taken together with the carbon atoms to which they are bound to form cyclopenten-1-yl. In certain embodiments, the present invention is directed to compounds of formula (I) wherein R² and R⁵ are taken together with the carbon atoms to which they are bound to form cyclopenten-1-yl. In certain embodiments, the present invention is directed to compounds of formula (I) wherein R³ and R⁴ are taken together with the carbon atoms to which they are bound to form cyclopenten-1-y.

In certain embodiments, the present invention is directed to any one or more compounds of formula (I) selected from the group consisting of
3-[4-[[5-(4-chlorophenyl)-2,3,6,7-tetrahydrooxepin-4-yl]methoxy]-2,3-dimethyl-phenyl]propanoic acid;
3-[4-[[5-(p-tolyl)-2,3,6,7-tetrahydrooxepin-4-yl]methoxy]-2-(trifluoromethyl)phenyl]propanoic acid;
3-[4-[(5-cyclopentyl-2,3,6,7-tetrahydrooxepin-4-yl)methoxy]-2-(trifluoromethyl)phenyl]propanoic acid;
3-[4-[[5-(4-chlorophenyl)-3,6-dihydro-2H-pyran-4-yl]methoxy]-2,3-dimethyl-phenyl]propanoic acid;
3-[4-[[5-(4-chlorophenyl)-3,6-dihydro-2H-pyran-4-yl]methoxy]-2-(trifluoromethyl)phenyl]propanoic acid;
3-[4-[[5-(p-tolyl)-3,6-dihydro-2H-pyran-4-yl]methoxy]-2-(trifluoromethyl)phenyl]propanoic acid;
3-[4-[(5-phenyl-3,6-dihydro-2H-pyran-4-yl)methoxy]-2-(trifluoromethyl)phenyl]propanoic acid;
3-[4-[(5-cylopentyl-3,6-dihydro-2H-pyran-4-yl)methoxy]-2-(trifluoromethyl)phenyl]propanoic acid;
3-[4-[[5-(3-fluoro-4-methyl-phenyl)-3,6-dihydro-2H-pyran-4-yl]methoxy]-2,3-dimethyl-phenyl]propanoic acid;
3-[4-[[4-(4-chlorophenyl)-3,6-dihydro-2H-pyran-5-yl]methoxy]-2,3-dimethyl-phenyl]propanoic acid;
3-[4-[[4-(4-chlorophenyl)-3,6-dihydro-2H-pyran-5-yl]methoxy]-2-(trifluoromethyl)phenyl]propanoic acid;
3-[4-[(4-ethyl-3,6-dihydro-2H-pyran-5-yl)methoxy]-2-(trifluoromethyl)phenyl]propanoic acid;
3-[2-chloro-4-[(4-cylopentyl-3,6-dihydro-2H-pyran-5-yl)methoxy]phenyl]propanoic acid;
3-[4-[(4-cyclopentyl-3,6-dihydro-2H-pyran-5-yl)methoxy]-2-(trifluoromethyl)phenyl]propanoic acid;

and pharmaceutically acceptable salts thereof.

In certain embodiments, the present invention is directed to compounds of formula (I) wherein a is 2, b is 1 and at least one of R², R³, R⁴ or R⁵ is other than hydrogen.

In certain embodiment, the present invention is directed to compounds of formula (I) wherein a is 2, b is 1 and wherein R¹ is selected from the group consisting of C₁₋₆alkyl, C₂₋₆-alkynyl, C₁₋₆cycloalkyl, —(C₁₋₂alkyl)-C₃₋₆cycloalkyl, C₅₋₆cycloalkenyl, 5,5-dimethyl-cyclopenten-1-yl, bicyclo[3.1.0]hexy-2-yl, (1S,4S)-2-methyl-bicyclo[2.2.1]hept-2-yl, (1R,2R,4S)-bicyclo[2.2.1]heptan-2-yl, 2,2-difluoro-benzo[d][1,3]dioxol-4-yl, —(C₁₋₂alkyl)-phenyl, —C(═CH₂)-phenyl, —C(O)-phenyl, biphenyl, tetrahydropyranyl, furanyl, thiazolyl, —(C₁₋₂alkyl)-thiazolyl, 3,6-dihydro-pyran-4-yl and 1-methyl-imidazol-4-yl; wherein the C₃₋₆cycloalkyl or C₅₋₆cycloalkenyl, whether alone or as part of a substituent group is optionally substituted with one or more (preferably one to two) substituents independently selected from the group consisting of halogen, C₁₋₄alkyl and fluorinated C₁₋₂alkyl; and wherein the furanyl, thiazolyl, or 3,6-dihydro-pyran-4-yl, whether alone or as part of a substituent group is optionally substituted with one or more (preferably one to three) substituents independently selected from the group consisting of halogen, hydroxy, cyano, C₁₋₄alkyl, fluorinated C₁₋₂alkyl, C₁₋₄alkoxy, fluorinated C₁₋₂alkoxy, —S—(C₁₋₂alkyl), —SO—(C₁₋₂alkyl), —SO₂—(C₁₋₂alkyl), nitro, —NR^{A}R^{B} and —NH—C(O)—(C₁₋₄alkyl); wherein R^{A} and R^{B} are each independently selected from the group consisting of hydrogen and C₁₋₄alkyl.

In certain embodiment, the present invention is directed to compounds of formula (I) wherein a is 2, b is 1 and wherein R¹ is selected from the group consisting of C₁₋₆alkyl, C₂₋₆-alkynyl, C₃₋₆cycloalkyl, —(C₁₋₂alkyl)-C₃₋₆cycloalkyl, C₅₋₆cycloalkenyl, 5,5-dimethyl-cyclopenten-1-yl, bicyclo[3.1.0]hexy-2-yl, (1S,4S)-2-methyl-bicyclo[2.2.1]hept-2-yl, (1R,2R,4S)-bicyclo[2.2.1]heptan-2-yl, 2,2-difluoro-benzo[d][1,3]dioxol-4-yl, —(C₁₋₂alkyl)-phenyl, —C(═CH₂)-phenyl, —C(O)-phenyl and tetrahydropyranyl; wherein the C₃₋₆cycloalkyl or C₅₋₆cycloalkenyl, whether alone or as part of a substituent group is optionally substituted with one or more (preferably one to two) substituents independently selected from the group consisting of halogen, C₁₋₄alkyl and fluorinated C₁₋₂alkyl.

In certain embodiment, the present invention is directed to compounds of formula (I) wherein a is 2, b is 1 and wherein R¹ is selected from the group consisting of C₁₋₆alkyl, C₂₋₆alkynyl, —(C₁₋₂alkyl)-C₃₋₆cycloalkyl, C₅₋₆cycloalkenyl, 5,5-dimethyl-cyclopenten-1-yl, bicyclo[3.1.0]hexy-2-yl, (1S,4S)-2-methyl-bicyclo[2.2.1]hept-2-yl, (1R,2R,4S)-bicyclo[2.2.1]heptan-2-yl, 2,2-difluoro-benzo[d][1,3]dioxol-4-yl, —(C₁₋₂alkyl)-phenyl, —C(═CH₂)-phenyl, —C(O)-phenyl and tetrahydropyranyl; wherein the C₃₋₆cycloalkyl or C₅₋₆cycloalkenyl, whether alone or as part of a substituent group is optionally substituted with one or more (preferably one to two) substituents independently selected from the group consisting of halogen, C₁₋₄alkyl and fluorinated C₁₋₂alkyl.

In certain embodiment, the present invention is directed to compounds of formula (I) wherein a is 1 and b is 1; or a is 1 and b is 2; or a is 2 and b is 2; provided that when a is 1 and b is 2, or when a is 2 and b is 2, then Z is —O—.

In certain embodiments, the present invention is directed to compounds of formula (I) wherein R¹ is selected from the group consisting of C₁₋₆alkyl and C₂₋₆-alkynyl.

In certain embodiments, the present invention is directed to compounds of formula (I) wherein at least one of R², R³, R⁴ or R⁵ is fluorinated C₁₋₂alkyl. In certain embodiments, the present invention is directed to compounds of formula (I) wherein R² is fluorinated C₁₋₂alkyl. In certain embodiments, the present invention is directed to compounds of formula (I) wherein R³ is fluorinated C₁₋₂alkyl. In certain embodiments, the present invention is directed to compounds of formula (I) wherein R⁴ is fluorinated C₁₋₂alkyl. In certain embodiments, the present invention is directed to compounds of formula (I) wherein R⁵ is fluorinated C₁₋₂alkyl.

In certain embodiments, the present invention is directed to compounds of formula (I) wherein at least one of R² is selected from the group consisting of fluorinated C₁₋₂alkyl, —(C₁₋₄alkyl)-S—(C₁₋₂alkyl), —(C₁₋₄alkyl)-SO—(C₁₋₂alkyl) and —(C₁₋₄alkyl)-SO₂—(C₁₋₂alkyl). In certain embodiments, the present invention is directed to compounds of formula (I) wherein R⁵ is selected from the group consisting of fluorinated C₁₋₂alkyl, vinyl, halogen substituted vinyl, ethynyl, hydroxy substituted C₁₋₂alkyl, C₁₋₄alkoxy, fluorinated C₁₋₂alkoxy and cyclopropyl-methyl-.

Additional embodiments of the present invention, include those wherein the substituents selected for one or more of the variables defined herein (i.e. a, b, Z, c, R⁰, R¹, R², R³, R⁴, R⁵, etc.) are independently selected to be any individual substituent or any subset of substituents selected from the complete list as defined herein.

In another embodiment of the present invention is any single compound or subset of compounds selected from the representative compounds listed in Tables 1-4, below.

Representative compounds of the present invention are as listed in Tables 1-4, below. Unless otherwise noted, wherein a stereogenic center is present in the listed compound, the compound was prepared as a mixture of stereo-isomers.

TABLE 1
Representative Compounds of Formula (I)

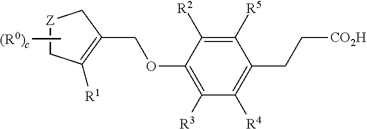

| ID | | | | | | | |
|---|---|---|---|---|---|---|---|
| No | Z | (R⁰)_{c} | R¹ | R² | R⁵ | R³ | R⁴ |
| 620 | O | c = 0 | phenyl | methyl | methyl | H | H |
| 621 | O | c = 0 | 4-chloro- | methyl | methyl | H | H |
| | | | phenyl | | | | |
| 625 | S | c = 0 | 4-fluoro- | methyl | H | H | H |
| | | | phenyl | | | | |
| 626 | S | c = 0 | 4-fluoro- | fluoro | H | fluoro | H |
| | | | phenyl | | | | |
| 627 | SO | c = 0 | 4-fluoro- | methyl | H | H | H |
| | | | phenyl | | | | |
| 628 | SO₂ | c = 0 | 4-fluoro- | fluoro | H | fluoro | H |
| | | | phenyl | | | | |
| 629 | SO₂ | c = 0 | 4-fluoro- | methyl | H | H | H |
| | | | phenyl | | | | |
| 630 | O | c = 0 | phenyl | methyl | H | H | H |
| 631 | O | c = 0 | cyclo- | H | trifluoro- | H | H |
| | | | pentyl | | methyl | | |
| 632 | O | c = 0 | cyclo- | H | H | H | H |
| | | | pentyl | | | | |
| 633 | O | c = 0 | cyclo- | H | methyl | H | H |
| | | | pentyl | | | | |
| 634 | O | c = 0 | cyclo- | H | fluoro | H | H |
| | | | pentyl | | | | |

TABLE 2
Representative Compounds of Formula (I)

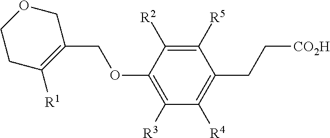

| ID No | (R⁰)_{c} | R¹ | R² | R⁵ | R³ | R⁴ |
|---|---|---|---|---|---|---|
| 640 | 6,6- | 4-chloro- | methyl | methyl | H | H |
| | dimethyl | phenyl | | | | |
| 641 | c = 0 | 4-chloro- | methyl | methyl | H | H |
| | | phenyl | | | | |
| 642 | c = 0 | 4-chloro- | fluoro | H | fluoro | H |
| | | phenyl | | | | |
| 645 | c = 0 | 4-chloro- | methyl | H | H | H |
| | | phenyl | | | | |
| 646 | c = 0 | 4-chloro | H | trifluoro- | H | H |
| | | phenyl | | methyl | | |
| 647 | c = 0 | 4-methoxy- | methyl | methyl | H | H |
| | | phenyl | | | | |
| 650 | c = 0 | 4-fluoro-phenyl | methyl | methyl | H | H |
| 651 | c = 0 | 2-fluoro-phenyl | methyl | methyl | H | H |
| 654 | c = 0 | pyrid-3-yl | methyl | methyl | H | H |
| 655 | c = 0 | 4-methyl- | methyl | methyl | H | H |
| | | phenyl | | | | |
| 656 | c = 0 | 3-methoxy- | methyl | methyl | H | H |
| | | phenyl | | | | |
| 658 | c = 0 | 3,4-difluoro- | methyl | methyl | H | H |
| | | phenyl | | | | |
| 659 | c = 0 | 3-fluoro-4- | methyl | methyl | H | H |
| | | methyl-phenyl | | | | |
| 660 | c = 0 | 2,4-difluoro- | methyl | methyl | H | H |
| | | phenyl | | | | |
| 661 | c = 0 | 3-methyl-4- | methyl | methyl | H | H |
| | | fluoro-phenyl | | | | |
| 662 | c = 0 | 2-methoxy-4- | methyl | methyl | H | H |
| | | chloro-phenyl | | | | |
| 663 | c = 0 | 3-fluoro-4- | methyl | methyl | H | H |
| | | chloro-phenyl | | | | |
| 664 | c = 0 | 3-methoxy-4- | methyl | methyl | H | H |
| | | chloro-phenyl | | | | |
| 665 | c = 0 | 4-methyl- | H | difluoro- | H | H |
| | | phenyl | | methyl | | |
| 666 | c = 0 | 4-methyl- | H | trifluoro- | H | trifluoro- |
| | | phenyl | | methyl | | methyl |
| 667 | c = 0 | 2-fluoro-4- | methyl | methyl | H | H |
| | | chloro-phenyl | | | | |
| 668 | c = 0 | 4-methyl- | H | trifluoro- | H | H |
| | | phenyl | | methyl | | |
| 669 | c = 0 | 2,6-dimethyl- | H | chloro | H | H |
| | | phenyl | | | | |
| 670 | c = 0 | 5,5-dimethyl- | H | trifluoro- | H | H |
| | | cyclo-penten- | | methyl | | |
| | | 1-yl | | | | |
| 671 | c = 0 | 2,6-dimethyl- | H | trifluoro- | H | H |
| | | phenyl | | methyl | | |
| 673 | c = 0 | 2,2-difluoro- | H | trifluoro- | H | H |
| | | benzo[d] | | methyl | | |
| | | [1,3]dioxol-4-yl | | | | |
| 674 | c = 0 | 2-trifluoro- | H | trifluoro- | H | H |
| | | methoxy- | | methyl | | |
| | | phenyl | | | | |
| 676 | c = 0 | 2-methoxy- | H | trifluoro- | H | H |
| | | phenyl | | methyl | | |
| 677 | c = 0 | 2-isopropyl- | H | trifluoro- | H | H |
| | | oxy-phenyl | | methyl | | |
| 678 | c = 0 | benzyl | H | trifluoro- | H | H |
| | | | | methyl | | |
| 679 | c = 0 | 3-methyl-4- | H | trifluoro- | H | H |
| | | chloro-phenyl | | methyl | | |
| 680 | c = 0 | 3,4-dichloro- | H | trifluoro- | H | H |
| | | phenyl | | methyl | | |
| 681 | c = 0 | 2,4,5-trimethyl- | H | trifluoro- | H | H |
| | | phenyl | | methyl | | |
| 682 | c = 0 | 2-methyl-4- | H | trifluoro- | H | H |
| | | chloro-phenyl | | methyl | | |
| 683 | c = 0 | 3,4-dimethyl- | H | trifluoro- | H | H |
| | | phenyl | | methyl | | |
| 684 | c = 0 | 2-fluoro-4- | H | trifluoro- | H | H |
| | | methyl-phenyl | | methyl | | |
| 685 | c = 0 | 3-fluoro-4- | H | trifluoro- | H | H |
| | | methyl-phenyl | | methyl | | |
| 686 | c = 0 | 4-trifluoro- | H | trifluoro- | H | H |
| | | methoxy- | | methyl | | |
| | | phenyl | | | | |
| 687 | c = 0 | 1-phenyl-vinyl | H | trifluoro- | H | H |
| | | | | methyl | | |
| 688 | c = 0 | benzyl | H | H | H | H |
| 691 | c = 0 | 4-methyl- | H | H | H | H |
| | | benzyl | | | | |
| 692 | c = 0 | 4-methyl- | H | trifluoro- | H | H |
| | | benzyl | | methyl | | |
| 695 | c = 0 | 4-chloro- | H | H | H | H |
| | | benzyl | | | | |
| 696 | c = 0 | 4-chloro- | H | trifluoro- | H | H |
| | | benzyl | | methyl | | |
| 697 | c = 0 | 4-methoxy- | H | H | H | H |
| | | benzyl | | | | |
| 698 | c = 0 | 4-methoxy- | H | trifluoro- | H | H |
| | | benzyl | | methyl | | |
| 699 | c = 0 | 1-phenyl-vinyl | H | H | H | H |
| 701 | c = 0 | 1-phenyl-ethyl | H | trifluoro- | H | H |
| | | | | methyl | | |
| 702 | c = 0 | 1-(4-fluoro- | H | H | H | H |
| | | phenyl)-vinyl | | | | |
| 703 | c = 0 | 1-(4-fluoro- | H | trifluoro- | H | H |
| | | phenyl)-vinyl | | methyl | | |
| 705 | c = 0 | 1-(4-trifluoro- | H | trifluoro- | H | H |
| | | methyl- | | methyl | | |
| | | phenyl)-vinyl | | | | |
| 706 | c = 0 | 1-(2-chloro- | H | trifluoro- | H | H |
| | | phenyl)-vinyl | | methyl | | |
| 707 | c = 0 | n-butyl | H | trifluoro- | H | H |
| | | | | methyl | | |
| 709 | c = 0 | 4-chloro- | H | chloro | H | H |
| | | benzyl | | | | |
| 713 | c = 0 | benzyl | H | chloro | H | H |
| 714 | c = 0 | benzyl | fluoro | H | fluoro | H |
| 716 | c = 0 | ethyl | H | trifluoro- | H | H |
| | | | | methyl | | |
| 717 | c = 0 | isobutyl | H | trifluoro- | H | H |
| | | | | methyl | | |
| 718 | c = 0 | cyclo-hexyl- | H | trifluoro- | H | H |
| | | methyl | | methyl | | |
| 719 | c = 0 | n-hexyl | H | trifluoro- | H | H |
| | | | | methyl | | |
| 720 | c = 0 | 1-phenyl-vinyl | H | chloro | H | H |
| 721 | c = 0 | 4-chloro- | fluoro | H | fluoro | H |
| | | benzyl | | | | |
| 722 | c = 0 | isopentyl | H | trifluoro- | H | H |
| | | | | methyl | | |
| 723 | c = 0 | 1-phenyl-vinyl | fluoro | H | H | H |
| 724 | c = 0 | cyclo-butyl | methyl | methyl | H | H |
| 725 | c = 0 | cyclo-butyl | H | chloro | H | H |
| 727 | c = 0 | cyclo-pentyl | H | chloro | H | H |
| 729 | c = 0 | cyclo-hexyl | H | trifluoro- | H | H |
| | | | | methyl | | |
| 731 | c = 0 | cyclo-pentyl | H | trifluoro- | H | H |
| | | | | methyl | | |
| 732 | c = 0 | isobutyl | H | chloro | H | H |
| 733 | c = 0 | isobutyl | fluoro | H | fluoro | H |
| 734 | c = 0 | 1-methyl-n- | H | trifluoro- | H | H |
| | | propyl | | methyl | | |
| 735 | c = 0 | isopropyl | H | trifluoro- | H | H |
| | | | | methyl | | |
| 736 | c = 0 | cyclo-pentyl | fluoro | H | fluoro | H |
| 737 | c = 0 | cyclo-pent-2- | H | chloro | H | H |
| | | en-1-yl | | | | |
| 738 | c = 0 | cyclo-pentyl | H | H | H | H |
| 739 | c = 0 | cyclo-pent-2- | H | trifluoro- | H | H |
| | | en-1-yl | | methyl | | |
| 740 | c = 0 | cyclo-pent-2- | H | H | H | H |
| | | en-1-yl | | | | |

TABLE 3
Representative Compounds of Formula (I)

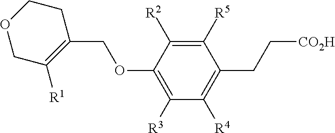

| ID | | | | | |
|---|---|---|---|---|---|
| No. | R¹ | R² | R⁵ | R³ | R⁴ |
| 745 | 4-chloro-phenyl | methyl | methyl | H | H |
| 752 | 4-chloro-phenyl | fluoro | H | fluoro | H |
| 753 | 4-chloro-phenyl | methyl | H | H | H |
| 754 | 4-chloro-phenyl | H | trifluoro- | H | H |
| | | | methyl | | |
| 755 | 4-chloro-phenyl | H | cyclo-propyl- | H | H |
| | | | methyl- | | |
| 756 | 4-methyl-phenyl | H | trifluoro- | H | H |
| | | | methyl | | |
| 757 | 2-phenyl-4-methyl- | H | trifluoro- | H | H |
| | phenyl | | methyl | | |
| 758 | 3-phenyl-4-methyl- | H | trifluoro- | H | H |
| | phenyl | | methyl | | |
| 759 | 2-fluoro-4-methyl- | H | trifluoro- | H | H |
| | phenyl | | methyl | | |
| 760 | 2,4-dimethyl-phenyl | H | trifluoro- | H | H |
| | | | methyl | | |
| 761 | 3-isopropyl-4- | H | trifluoro- | H | H |
| | methyl-phenyl | | methyl | | |
| 762 | 2-isopropyl-4- | H | trifluoro- | H | H |
| | methyl-phenyl | | methyl | | |
| 763 | phenyl | H | trifluoro- | H | H |
| | | | methyl | | |
| 764 | cyclo-pentyl | H | H | H | H |
| 765 | cyclo-pentyl | H | trifluoro- | H | H |
| | | | methyl | | |
| 766 | 3-fluoro-4-methyl- | methyl | methyl | H | H |
| | phenyl | | | | |
| 767 | 3-fluoro-4-methyl- | fluoro | H | fluoro | H |
| | phenyl | | | | |
| 768 | 3-fluoro-4-methyl- | fluoro | fluoro | H | H |
| | phenyl | | | | |
| 769 | 3-fluoro-4-methyl- | H | H | H | H |
| | phenyl | | | | |

TABLE 4
Representative Compounds of Formula (I)

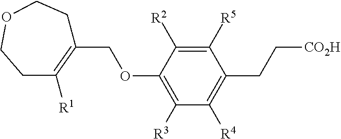

| ID | | | | | |
|---|---|---|---|---|---|
| No | R¹ | R² | R⁵ | R³ | R⁴ |
| 775 | 4-chloro-phenyl | methyl | methyl | H | H |
| 776 | 4-chloro-phenyl | H | trifluoro- | H | H |
| | | | methyl | | |
| 777 | 4-methyl-phenyl | H | trifluoro- | H | H |
| | | | methyl | | |
| 778 | 3-fluoro-4-methyl- | fluoro | fluoro | H | H |
| | phenyl | | | | |
| 779 | 3-fluoro-4-methyl- | H | trifluoro- | H | H |
| | phenyl | | methyl | | |
| 780 | cyclo-pentyl | H | H | H | H |
| 781 | 3-fluoro-4-methyl- | fluoro | H | H | methyl |
| | phenyl | | | | |
| 783 | 3-fluoro-4-methyl- | H | H | H | H |
| | phenyl | | | | |
| 784 | cyclo-pentyl | H | trifluoro- | H | H |
| | | | methyl | | |
| 785 | 3-fluoro-4-methyl- | fluoro | H | fluoro | H |
| | phenyl | | | | |
| 786 | cyclo-pentyl | H | chloro | H | H |
| 787 | 3-fluoro-4-methyl- | methyl | methyl | H | H |
| | phenyl | | | | |

In an embodiment, the present invention is directed to a compound of formula (I); wherein the compound of formula exhibits an EC₅₀ against GPR120, measured as described in the Biological Examples, which follow herein, of less than about 1.0 NM, preferably less than about 0.500 μM, more preferably less than about 0.250 μM, more preferably less than about 0.100 μM, more preferably less than about 0.050 μM, more preferably less than about 0.025 μM.

In an embodiment, the present invention is directed to a compound of formula (I); wherein the compound of formula exhibits an EC₅₀ against GPR40, measured as described in the Biological Examples, which follows herein, of less than about 1.0 μM, preferably less than about 0.500 μM, more preferably less than about 0.250 μM, more preferably less than about 0.100 μM, more preferably less than about 0.050 μM.

### Definitions

As used herein, unless otherwise noted, the term “halogen” shall mean chlorine, bromine, fluorine and iodine.

As used herein, unless otherwise noted, the term “oxo” shall mean an oxygen atom bound through a double bond (i.e ═O).

As used herein, unless otherwise noted, the term “alkyl” whether used alone or as part of a substituent group, include straight and branched chains. For example, alkyl radicals include methyl, ethyl, propyl, isopropyl, butyl, isobutyl, sec-butyl, t-butyl, pentyl and the like. The term “C_{X-Y}alkyl” wherein X and Y are integers, whether used alone or as part of a substituent group, include straight and branched chains of between X and Y carbon atoms. For example, the term “C₁₋₄alkyl” includes straight and branched chains of between 1 and 4 carbon atoms, including methyl, ethyl, n-propyl, isopropyl, n-butyl, isobutyl and tert-butyl.

One skilled in the art will recognize that the term “—(C_{X-Y}alkyl)-” shall denote any C₁₋₄alkyl carbon chain as herein defined, wherein said C_{X-Y}alkyl chain is divalent and is further bound through two points of attachment, preferably through two terminal carbon atoms.

As used herein, unless otherwise noted, the term “fluorinated C_{X-Y}alkyl” shall mean any C_{X-Y}alkyl group as defined above substituted with at least one fluoro atom. For example, the term fluorinated C₁₋₄alkyl shall mean any C₁₋₄alkyl group as defined above substituted with at least one, preferably one to five, more preferably one to three fluoro atoms. Suitable examples of fluorinated C_{X-Y}alkyl groups include, but are not limited to —CF₃, —CH₂—CF₃, —CF₂CF₃, —CF₂—CF₂—CF₂—CF₃, and the like.

As used herein, unless otherwise noted, the term “hydroxy substituted C_{X-Y}alkyl” shall mean a C_{X-Y}alkyl group as defined above substituted with at least one hydroxy group. Preferably, the C_{X-Y}alkyl group is substituted with one hydroxy group. Preferably, the C_{X-Y}alkyl group is substituted with a hydroxy group at the terminal carbon. Suitable examples include, but are not limited to, —CH₂(OH), —CH₂—CH₂(OH), —CH₂—CH(OH)—CH₂, and the like.

As used herein, unless otherwise noted, the term “halogen substituted vinyl” shall mean a vinyl (i.e —CH═CH₂) group substituted with one or more, preferably one to two, more preferably one independently selected halogen atoms as defined above. Preferably, the halogen substituted vinyl is substituted with one fluoro, chloro, bromo or iodo group. More preferably the halogen substituted vinyl is a bromo substituted vinyl.

As used herein, unless otherwise noted, the term “C_{X-Y}alkynyl” shall mean a straight or branched chain of between X and Y carbon atoms, wherein the straight or branched chain contains as least one, preferably one, unsaturated triple bond. For example, the term “C₂₋₆alkynyl” includes straight and branched chains of between 2 and 6 carbon atoms containing at least one, preferably one, unsaturated triple bond such as ethynyl, n-propyn-1-yl, n-pentyn-1-yl, pentyn-2-yl, and the like.

As used herein, unless otherwise noted, “alkoxy” shall denote an oxygen ether radical of the above described straight or branched chain alkyl groups. For example, methoxy, ethoxy, n-propoxy, sec-butoxy, t-butoxy, n-hexyloxy and the like. The term “C_{X-Y}alkoxy” wherein X and Y are integers, whether used alone or as part of a substituent group, shall denote an oxygen ether radical of the above described straight or branched chain of between X and Y carbon atoms. For example, the term “C₁₋₄alkoxy” includes oxygen ether radicals of straight and branched chains of between 1 and 4 carbon atoms, including methoxy, ethoxy, n-propoxy, isopropoxy, n-butoxy, isobutoxy and tert-butoxy.

As used herein, unless otherwise noted, the term “fluorinated C_{X-Y}alkoxy” shall mean any C_{X-Y}alkoxy group as defined above substituted with at least one fluoro atom. For example, the term fluorinated C₁₋₄alkoxy shall mean any C₁₋₄alkoxy group as defined above substituted with at least one, preferably one to five, more preferably one to three fluoro atoms. Suitable examples of fluorinated C_{X-Y}alkoxy groups include, but are not limited to —OCF₃, —OCH₂—CF₃, —CF₂CF₃, —OCF₂—CF₂—CF₂—CF₃, and the like.

As used herein, unless otherwise noted, the term “C₃₋₆cycloalkyl” shall mean any stable 3-8 membered monocyclic, saturated ring system, for example cyclopropyl, cyclobutyl, cyclopentyl, cyclohexyl, cycloheptyl and cyclooctyl. Similarly, the term “C₅₋₆cycloalkyl” shall mean any stable 5-6 membered monocyclic, saturated ring system, more particularly cyclopentyl and cyclohexyl.

As used herein, unless otherwise noted, the term “C₅₋₆cycloalkenyl” shall mean any stable 5-6 membered monocyclic, partially unsaturated ring system, wherein the partially unsaturated ring system contains one to two, preferably one unsaturated double bond. Suitably examples include, but are not limited to cyclopentenyl and cyclohexenyl.

When a particular group is “substituted” (e.g., alkyl, cycloalkyl, phenyl, pyridyl, etc.), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

As used herein, the notation “*” shall denote the presence of a stereogenic center.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, wherein the compound is present as a diastereomer, the diastereomer is present at an diastereomeric excess of greater than or equal to about 80%, more preferably, at an diastereomeric excess of greater than or equal to about 90%, more preferably still, at an diastereomeric excess of greater than or equal to about 95%, more preferably still, at an diastereomeric excess of greater than or equal to about 98%, most preferably, at an diastereomeric excess of greater than or equal to about 99%.

Furthermore, some of the crystalline forms for the compounds of the present invention may exist as polymorphs and as such are intended to be included in the present invention. In addition, some of the compounds of the present invention may form solvates with water (i.e., hydrates) or common organic solvents, and such solvates are also intended to be encompassed within the scope of this invention.

As used herein, unless otherwise noted, the term “isotopologues” shall mean molecules that differ only in their isotopic composition. More particularly, an isotopologue of a molecule differs from the parent molecule in that it contains at least one atom which is an isotope (i.e. has a different number of neutrons from its parent atom).

For example, isotopologues of water include, but are not limited to, “light water” (HOH or H₂O), “semi-heavy water” with the deuterium isotope in equal proportion to protium (HDO or ¹H²HO), “heavy water” with two deuterium isotopes of hydrogen per molecule (D₂O or ²H₂O), “super-heavy water” or tritiated water (T₂O or ³H₂O), where the hydrogen atoms are replaced with tritium (³H) isotopes, two heavy-oxygen water isotopologues (H₂¹⁸O and H₂¹⁷O) and isotopologues where the hydrogen and oxygen atoms may each independently be replaced by isotopes, for example the doubly labeled water isotopologue D₂¹⁸O.

It is intended that within the scope of the present invention, any one or more element(s), in particular when mentioned in relation to a compound of formula (I), shall comprise all isotopes and isotopic mixtures of said element(s), either naturally occurring or synthetically produced, either with natural abundance or in an isotopically enriched form. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O. The isotopes may be radioactive or non-radioactive. Radiolabelled compounds of formula (I) may comprise one or more radioactive isotope(s) selected from the group of ³H, ¹¹C, ¹⁸F, ¹²²I, ¹²³I, ¹²⁵I, ¹³¹I, ⁷⁵Br, ⁷⁶Br, ⁷⁷Br and ⁸²Br. Preferably, the radioactive isotope is selected from the group of ³H, ¹¹C and ¹⁸F.

Under standard nomenclature used throughout this disclosure, the terminal portion of the designated side chain is described first, followed by the adjacent functionality toward the point of attachment. Thus, for example, a “phenylC₁-C₆alkylaminocarbonylC₁-C₆alkyl” substituent refers to a group of the formula

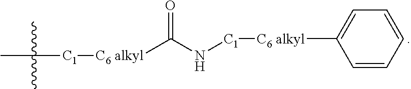

Throughout the specification, wherein the

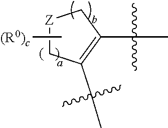

(heterocycloalkenyl) group is named or listed as a separate substituent group—as a substituent group named independent of the groups bound to the carbon atoms of the heterocycloalkenyl double bond, (e.g. in the listing of the heterocycloalkenyl group in the tables of compounds, embodiments, etc.)—said heterocycloalkenyl group shall be named using standard IUPAC heterocycle nomenclature. For example, wherein the heterocycloalkenyl group is listed or named as 3,6-dihydro-2H-pyranyl, the ring carbon positions shall be numbered as indicated below:

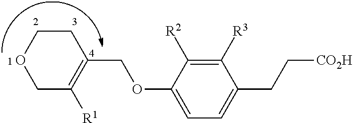

wherein the 1-position is defined as the position of the heteroatom and the numbering proceeds in a direction such that the carbon atom bound to the —CH₂—O— portion of the compound of formula (I) has priority in numbering over the carbon bound to the R¹ group.

One skilled in the art will recognize that when the complete compound of formula (I) containing said heterocycloalkenyl group is named according to standard naming convention, the numbering (defined position of the heteroatom or any R⁰ group(s)) may be different from the numbering used in naming the heterocycloalkenyl group independently, as a result of the presence and chemical structure of the R¹ substituent group, which may change the numbering priority.

Abbreviations used in the specification, particularly the Schemes and Examples, are as follows:

| | | |
|---|---|---|
| ADDP | = | 1,1′-(Azodicarbonyl)dipiperidine |
| aq. | = | Aqueous |
| BF₃ • EtO₂ | = | Boron Trifluoride Diethyl Etherate |
| BSA | = | Bovine Serum Albumin |
| Bu₃P or n-Bu₃P | = | Tri(n-butyl)phosphine |
| DCM | = | Dichloromethane |
| DEAD | = | Diethylazodicarboxylate |
| DIAD | = | Diisopropylazodicarboxylate |
| DIBAL or DIBAL-H | = | Diisobutylaluminum hydride |
| DIO | = | Diet-induced obese/obesity |
| DIPEA or DIEA | = | Diisopropylethylamine |
| DME | = | Dimethoxyethane |
| DMEM | = | Dulbecco's Modified Eagle Medium |
| DMF | = | N,N-Dimethylformamide |
| DMSO | = | Dimethylsulfoxide |
| dppf | = | 1,1′-Ferrocenediyl-bis(diphenylphosphine) |
| EA | = | Ethyl Acetate |
| ESI | = | Electrospray ionization |
| EtOAc | = | Ethyl acetate |
| FBS | = | Fetal Bovine Serum |
| FLIPR | = | Fluorometric imaging Plate Reader |
| HBSS | = | Hank's Balanced Salt Solution |
| HDL | = | High Density Lipoproetin |
| HEPES | = | 4-(2-Hydroxyethyl)-1-Piperizine |
| | | Ethane Sulfonic Acid |
| ¹H NMR | = | ¹Hydrogen Nuclear Magnetic Resonance |
| HPLC | = | High Pressure Liquid Chromatography |
| i.p. | = | Intra-peritoneal |
| IPGTT | = | Intra-peritoneal Glucose Tolerance Test |
| LAH | = | Lithium Aluminum Hydride |
| LDL | = | Low Density Lipoprotein |
| MeOH | = | Methanol |
| Mesyl | = | Methylsulfonyl |
| MS | = | Mass Spectroscopy |
| MTBE | = | Methyl t-Butyl Ether |
| NASH | = | Non-alcoholic Steatohepatitis |
| NAFLD | = | Non-alcoholic Fatty Liver Disease |
| OGTT | = | Oral Glucose Tolerance Test |
| OTf | = | Triflate (i.e CF₃SO₃-) |
| Pd₂(OAc)₂ | = | Palladium(II)acetate |
| Pd₂(dba)₃ | = | Tris(dibenzylidene acetone)dipalladium(0) |
| Pd(dppf)Cl₂ | = | [1,1′-Bis(diphenylphosphino)ferrocene] |
| | | dichloropalladium(II) |
| Pd(PPh₃)₄ | = | Tetrakistriphenylphosphine palladium (0) |
| Pd(PPh₃)₂Cl₂ | = | Bis(triphenylphosphine)palladium (II) chloride |
| PPh₃ or TPP | = | Triphenyl Phosphine |
| Rochelle's salt | = | Potassium sodium tartrate tetrahydrate |
| RuPhos | = | 2-Dicyclohexylphosphino-2′,6′- |
| | | diisopropoxybiphenyl |
| sat. | = | saturated |
| SPhos | = | 2-Dicyclohexylphosphino-2′,6′- |
| | | dimethoxybiphenyl |
| t-BOC or Boc | = | Tert-Butoxycarbonyl |
| TEA | = | Triethylamine |
| THP | = | Tetrahydropyranyl |
| TFA | = | Trifluoroacetic Acid |
| TfO₂ | = | Triflic Anhydride |
| THF | = | Tetrahydrofuran |
| TLC | = | Thin Layer Chromatography |
| TMS | = | Trismethylsilyl |
| Tosyl | = | p-Toluenesulfonyl |

As used herein, unless otherwise noted, the term “isolated form” shall mean that the compound is present in a form which is separate from any solid mixture with another compound(s), solvent system or biological environment. In an embodiment of the present invention, the compound of formula (I) is present in an isolated form.

As used herein, unless otherwise noted, the term “substantially pure form” shall mean that the mole percent of impurities in the isolated compound is less than about 5 mole percent, preferably less than about 2 mole percent, more preferably, less than about 0.5 mole percent, most preferably, less than about 0.1 mole percent. In an embodiment of the present invention, the compound of formula (I) is present as a substantially pure form.

As used herein, unless otherwise noted, the term “substantially free of a corresponding salt form(s)” when used to described the compound of formula (I) shall mean that mole percent of the corresponding salt form(s) in the isolated base of formula (I) is less than about 5 mole percent, preferably less than about 2 mole percent, more preferably, less than about 0.5 mole percent, most preferably less than about 0.1 mole percent. In an embodiment of the present invention, the compound of formula (I) is present in a form which is substantially free of corresponding salt form(s).

As used herein unless otherwise noted, the term “cardiovascular disorders” shall mean any cardiovascular disease, disorder or condition in which obesity and/or diabetes (preferably, Type II Diabetes Mellitus) has a role in the initiation or exacerbation of said disorder or condition. Suitable examples include, but are not limited to, hypertension, atherosclerosis, and cardiac fibrosis.

For purposes of the present invention, the term “modulated by the GPR120 receptor” is used to refer to the condition of being affected by the modulation of the GPR120 receptor, including but not limited to, the state of being mediated by activation or agonism of the GPR120 receptor.

For purposes of the present invention, the term “modulated by the GPR40 receptor” is used to refer to the condition of being affected by the modulation of the GPR40 receptor, including but not limited to, the state of being mediated by activation or agonism of the GPR40 receptor.

As used herein, unless otherwise noted, the term “disorder modulated by the GPR120 receptor” shall mean any disease, disorder or condition characterized in that at least one of its characteristic symptoms is alleviated or eliminated upon treatment with a GPR120 receptor agonist. Suitably examples include, but are not limited to obesity, obesity induced inflammation, impaired glucose tolerance, elevated fasting glucose, insulin resistance, hyperglycemia, hyperinsulinemia, Type II Diabetes Mellitus, metabolic syndrome (also known as Syndrome X), gestational diabetes, diabetic retinopathy, kidney disease, ketoacidosis, diabetic nephropathy, dyslipidemia, elevated LDL, hyperipidemia, hyperlipoproteinemia, hypertriglyceridemia (i.e. elevated triglycerides), non-alcoholic steatohepatitis (NASH), non-alcoholic fatty liver disease (NAFLD) and liver fibrosis.

Preferably, the disorder modulated by the GPR120 receptor is selected from the group consisting of obesity, impaired glucose tolerance, elevated fasting glucose, insulin resistance, hyperglycemia, hyperinsulinemia, Type II Diabetes Mellitus, metabolic syndrome (also known as Syndrome X), diabetic retinopathy, diabetic nephropathy, dyslipidemia, elevated LDL, hyperipidemia, hypertriglyceridemia (i.e. elevated triglycerides), non-alcoholic steatohepatitis (NASH) and non-alcoholic fatty liver disease (NAFLD). More preferably, the disorder modulated by the GPR120 receptor is selected from the group consisting of obesity, Type II diabetes, metabolic syndrome (also known as Syndrome X), dyslipidemia. hypertriglyceridemia (i.e. elevated triglycerides), non-alcoholic steatohepatitis (NASH) and non-alcoholic fatty liver disease (NAFLD). More preferably, the disorder modulated by the GPR120 receptor is selected from the group consisting of obesity, Type II diabetes, metabolic syndrome (also known as Syndrome X), dyslipidemia and hypertriglyceridemia.

As used herein, unless otherwise noted, the term “disorder modulated by the GPR40 receptor” shall mean any disease, disorder or condition characterized in that at least one of its characteristic symptoms is alleviated or eliminated upon treatment with a GPR40 receptor agonist. Suitably examples include, but are not limited to obesity, obesity induced inflammation, impaired glucose tolerance, elevated fasting glucose, insulin resistance, hyperglycemia, hyperinsulinemia, Type II Diabetes Mellitus, metabolic syndrome (also known as Syndrome X), gestational diabetes, diabetic retinopathy, kidney disease, ketoacidosis, diabetic nephropathy, dyslipidemia, elevated LDL, hyperipidemia, hyperlipoproteinemia, hypertriglyceridemia (i.e. elevated triglycerides), non-alcoholic steatohepatitis (NASH), non-alcoholic fatty liver disease (NAFLD), liver fibrosis and cardiovascular disorders (including but not limited to hypertension, atherosclerosis, thrombotic disorders, and cardiac fibrosis).

Preferably, the disorder modulated by the GPR40 receptor is selected from the group consisting of obesity, impaired glucose tolerance, elevated fasting glucose, insulin resistance, hyperglycemia, hyperinsulinemia, Type II Diabetes Mellitus, metabolic syndrome (also known as Syndrome X), diabetic retinopathy, diabetic nephropathy, dyslipidemia, elevated LDL, hyperipidemia, hypertriglyceridemia (i.e. elevated triglycerides), non-alcoholic steatohepatitis (NASH) and non-alcoholic fatty liver disease (NAFLD). More preferably, the disorder modulated by the GPR40 receptor is selected from the group consisting of obesity, Type II diabetes, metabolic syndrome (also known as Syndrome X), dyslipidemia. hypertriglyceridemia (i.e. elevated triglycerides), non-alcoholic steatohepatitis (NASH) and non-alcoholic fatty liver disease (NAFLD). More preferably, the disorder modulated by the GPR40 receptor is selected from the group consisting of obesity, Type II diabetes, metabolic syndrome (also known as Syndrome X), dyslipidemia and hypertriglyceridemia

For purposes of the present invention, the terms “modulated by the GPR120 and GPR40 receptor”, “responds to dual agonism of the GPR120 and GPR40 receptors” and “responds to agonism of both the GPR120 and GPR40 receptors” are used to refer to the condition of being affected by the modulation of both the GPR120 and the GPR40 receptor, including but not limited to, the state of being mediated by the activation or agonism of both the GPR120 and the GPR40 receptor.

As used herein, unless otherwise noted, the terms “modulated by the GPR120 and GPR40 receptor”, “responds to dual agonism of the GPR120 and GPR40 receptors” and “responds to agonism of both the GPR120 and GPR40 receptors” shall mean any disease, disorder or condition characterized in that at least one of its characteristic symptoms is alleviated or eliminated upon treatment with a dual GPR120 and GPR40 receptor agonist. Suitably examples include, but are not limited to obesity, obesity induced inflammation, impaired glucose tolerance, elevated fasting glucose, insulin resistance, hyperglycemia, hyperinsulinemia, Type II Diabetes Mellitus, metabolic syndrome (also known as Syndrome X), gestational diabetes, diabetic retinopathy, kidney disease, ketoacidosis, diabetic nephropathy, dyslipidemia, elevated LDL, hyperlipidemia, hyperlipoproteinemia, hypertriglyceridemia (i.e. elevated triglycerides), non-alcoholic steatohepatitis (NASH), non-alcoholic fatty liver disease (NAFLD), liver fibrosis and cardiovascular disorders (including but not limited to hypertension, atherosclerosis, thrombotic disorders, and cardiac fibrosis).

Preferably, the disorder modulated by dual agonism of the GPR120 and GPR40 receptors is selected from the group consisting of obesity, impaired glucose tolerance, elevated fasting glucose, insulin resistance, hyperglycemia, hyperinsulinemia, Type II Diabetes Mellitus, metabolic syndrome (also known as Syndrome X), diabetic retinopathy, diabetic nephropathy, dyslipidemia, elevated LDL, hyperlipidemia, hypertriglyceridemia (i.e. elevated triglycerides), non-alcoholic steatohepatitis (NASH) and non-alcoholic fatty liver disease (NAFLD). More preferably, the disorder modulated by dual agonism of the GPR120 and GPR40 receptors is selected from the group consisting of obesity, Type II diabetes, metabolic syndrome (also known as Syndrome X), dyslipidemia. hypertriglyceridemia (i.e. elevated triglycerides), non-alcoholic steatohepatitis (NASH) and non-alcoholic fatty liver disease (NAFLD). More preferably, the disorder modulated by dual agonism of the GPR120 and GPR40 receptors is selected from the group consisting of obesity, Type II diabetes, metabolic syndrome (also known as Syndrome X), dyslipidemia and hypertriglyceridemia

In certain embodiments of the present invention, the disorder modulated by a dual GPR120 and GPR40 agonist is selected from the group consisting of obesity, hyperglycemia, impaired glucose tolerance, elevated fasting glucose, insulin resistance, hyperinsulinemia, Type II Diabetes Mellitus, metabolic syndrome (also known as Syndrome X), dyslipidemia, hyperlipoproteinemia, hyperlipidemia, elevated LDL, hypertriglyceridemia (i.e. elevated triglycerides), kidney disease, ketoacidosis, diabetic neuropathy and diabetic retinopathy.

In certain embodiments of the present invention, the disorder modulated by a dual GPR120 and GPR40 agonist is selected from the group consisting of obesity, hyperglycemia, impaired glucose tolerance, elevated fasting glucose, insulin resistance, hyperinsulinemia, Type II Diabetes Mellitus, metabolic syndrome (also known as Syndrome X), kidney disease, ketoacidosis, diabetic neuropathy and diabetic retinopathy. Preferably, the disorder modulated by a dual GPR120 and GPR40 agonist is selected from the group consisting of hyperglycemia, impaired glucose tolerance, elevated fasting glucose, insulin resistance, hyperinsulinemia and Type II Diabetes Mellitus.

As used herein, unless otherwise noted, the terms “treating”, “treatment” and the like, shall include the management and care of a subject or patient (preferably mammal, more preferably human) for the purpose of combating a disease, condition, or disorder and includes the administration of a compound of the present invention to prevent the onset of the symptoms or complications, alleviate the symptoms or complications (including, to reduce the frequency or severity of one or more symptoms), or eliminate the disease, condition, or disorder.

As used herein, unless otherwise noted, the term “prevention” shall include (a) the delay or avoidance of the development of additional symptoms; and/or (b) delay or avoidance of the development of the disorder or condition along a known development pathway.

One skilled in the art will recognize that wherein the present invention is directed to methods of prevention, a subject in need of thereof (i.e. a subject in need of prevention) shall include any subject or patient (preferably a mammal, more preferably a human) who has experienced or exhibited at least one symptom of the disorder, disease or condition to be prevented. A subject in need thereof may additionally be a subject (preferably a mammal, more preferably a human) who has not exhibited any symptoms of the disorder, disease or condition to be prevented, but who has been deemed by a physician, clinician or other medical profession to be at risk of developing said disorder, disease or condition. For example, the subject may be deemed at risk of developing a disorder, disease or condition (and therefore in need of prevention or preventive treatment) as a consequence of the subject's medical history, including, but not limited to, family history, pre-disposition, co-existing (comorbid) disorders or conditions, genetic testing, and the like.

The term “subject” as used herein, refers to an animal, preferably a mammal, most preferably a human, who has been the object of treatment, observation or experiment. Preferably, the subject has experienced and/or exhibited at least one symptom of the disease or disorder to be treated and/or prevented.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

As more extensively provided in this written description, terms such as “reacting” and “reacted” are used herein in reference to a chemical entity that is any one of: (a) the actually recited form of such chemical entity, and (b) any of the forms of such chemical entity in the medium in which the compound is being considered when named.

One skilled in the art will recognize that, where not otherwise specified, the reaction step(s) is performed under suitable conditions, according to known methods, to provide the desired product. One skilled in the art will further recognize that, in the specification and claims as presented herein, wherein a reagent or reagent class/type (e.g. base, solvent, etc.) is recited in more than one step of a process, the individual reagents are independently selected for each reaction step and may be the same of different from each other. For example wherein two steps of a process recite an organic or inorganic base as a reagent, the organic or inorganic base selected for the first step may be the same or different than the organic or inorganic base of the second step. Further, one skilled in the art will recognize that wherein a reaction step of the present invention may be carried out in a variety of solvents or solvent systems, said reaction step may also be carried out in a mixture of the suitable solvents or solvent systems.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein.

Examples of suitable solvents, bases, reaction temperatures, and other reaction parameters and components are provided in the detailed descriptions which follow herein. One skilled in the art will recognize that the listing of said examples is not intended, and should not be construed, as limiting in any way the invention set forth in the claims which follow thereafter.

As used herein, unless otherwise noted, the term “aprotic solvent” shall mean any solvent that does not yield a proton. Suitable examples include, but are not limited to DMF, 1,4-dioxane, THF, acetonitrile, pyridine, dichloroethane, dichloromethane, MTBE, toluene, acetone, and the like.

As used herein, unless otherwise noted, the term “leaving group” shall mean a charged or uncharged atom or group which departs during a substitution or displacement reaction. Suitable examples include, but are not limited to, Br, Cl, I, mesylate, tosylate, and the like.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis. John Wiley & Sons, 1991. The protecting groups may be removed at a convenient subsequent stage using methods known from the art.

As used herein, unless otherwise noted, the term “nitrogen protecting group” shall mean a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, t-butyl, benzyl, phenylethyl, CH₂═CH—CH₂—, and the like; amides—groups of the formula —C(O)—R′ wherein R′ is for example methyl, phenyl, trifluoromethyl, and the like; N-sulfonyl derivatives—groups of the formula —SO₂—R″ wherein R″ is for example tolyl, phenyl, trifluoromethyl, 2,2,5,7,8-pentamethylchroman-6-yl-, 2,3,6-trimethyl-4-methoxybenzene, and the like. Other suitable nitrogen protecting groups may be found in texts such as T. W. Greene & P. G. M. Wuts, Protective Groups in Organic Synthesis. John Wiley & Sons, 1991.

As used herein, unless otherwise noted, the term “oxygen protecting group” shall mean a group which may be attached to a oxygen atom to protect said oxygen atom from participating in a reaction and which may be readily removed following the reaction. Suitable oxygen protecting groups include, but are not limited to, acetyl, benzoyl, t-butyl-dimethylsilyl, trimethylsilyl (TMS), MOM, THP, and the like. Other suitable oxygen protecting groups may be found in texts such as T. W. Greene & P. G. M. Wuts, Protective Groups in Organic Synthesis, John Wiley & Sons, 1991.

One skilled in the art will recognize that wherein a reaction step of the present invention may be carried out in a variety of solvents or solvent systems, said reaction step may also be carried out in a mixture of the suitable solvents or solvent systems.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

Additionally, chiral HPLC against a standard may be used to determine percent enantiomeric excess (% ee). The enantiomeric excess may be calculated as follows

[(Rmoles−Smoles)/(Rmoles+Smoles)]×100%

where Rmoles and Smoles are the R and S mole fractions in the mixture such that Rmoles+Smoles=1. The enantiomeric excess may alternatively be calculated from the specific rotations of the desired enantiomer and the prepared mixture as follows:

ee=([α−obs]/[α−max])×100.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids which may be used in the preparation of pharmaceutically acceptable salts include, but are not limited to, the following: acids including acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cycamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hipuric acid, hydrobromic acid, hydrochloric acid, (+)-L-lactic acid, (±)-DL-lactic acid, lactobionic acid, maleic acid, (−)-L-malic acid, malonic acid, (t)-DL-mandelic acid, methanesulfonic acid, naphthalene-2-sulfonic acid, naphthalene-1,5-disulfonic acid, 1-hydroxy-2-naphthoic acid, nicotinc acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid.

Representative bases which may be used in the preparation of pharmaceutically acceptable salts include, but are not limited to, the following: bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

### General Synthesis Schemes

Compounds of formula (I) wherein a is an integer from 1 to 2 and wherein b is an integer from 1 to 2 may be prepared according to the process outlined in Scheme 1.

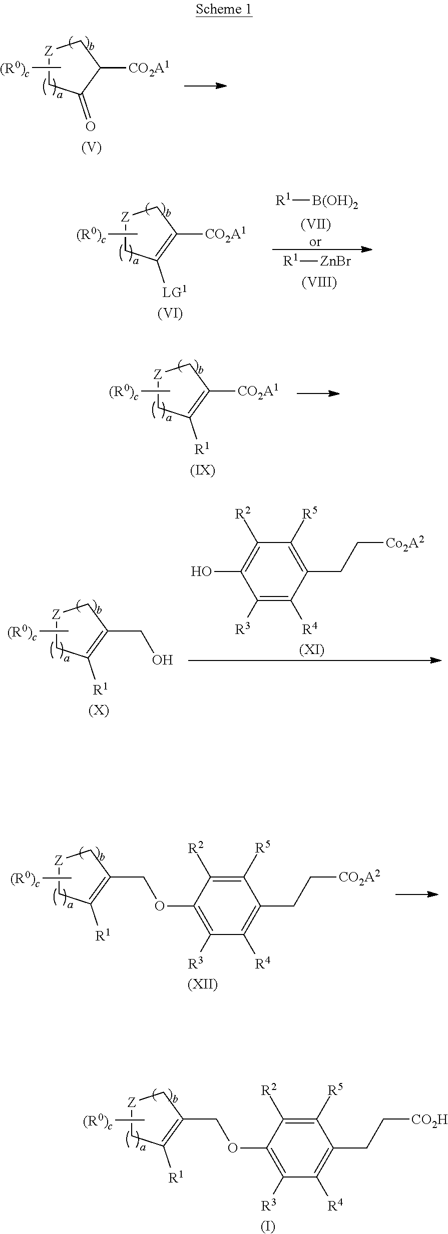

Accordingly, a suitably substituted compound of formula (V), wherein A¹ is selected from the group consisting of C₁₋₄alkyl, preferably methyl or ethyl, a known compound or compound prepared by known methods, is reacted with a suitably selected triflating agent such as triflic anhydride, nonaflate (i.e. SO₂C₄F₉), and the like, a known compound; in the presence of a suitably selected base such as NaH, TEA, DIPEA and the like; in a suitably selected solvent (other than THF) such as diethyl ether, MTBE, di-t-butyl ether, DCM, and the like; to yield the corresponding compound of formula (VI), wherein LG¹ is —OTf. Alternatively, the compound of formula (V) is reacted with N-phenyl-bis(trifluoromethanesulfonimide), a known compound; in the presence of a suitably selected base such as NaH, DIPEA, TEA, and the like; in a suitably selected solvent (other than THF) such as DMF, MTBE, diethyl ether, di(t-butyl)ether, and the like; to yield the corresponding compound of formula (VI), wherein LG¹ is —OTf.

The compound of formula (VI) is reacted with a suitably substituted boronic acid, a compound of formula (VII), a known compound or compound prepared by known methods; in the presence of a suitably selected catalyst such as Pd(OAc)₂, Pd(PPh₃)₄, Pd₂(dba)₃.CH₂Cl₂, and the like; in the presence of a suitably selected ligand such as SPhos, PPh₃, dppf, and the like; in the presence of a suitably selected base such as Cs₂CO₃, K₂CO₃, K₃PO₄, and the like; in a suitably selected organic solvent such as 1,4-dioxane, toluene, DME, THF, and the like; to yield the corresponding compound of formula (IX).

Alternatively, the compound of formula (VI) is reacted with a suitably substituted zinc bromide, a compound of formula (VIII); in the presence of a suitably selected coupling catalyst such as Pd(PPh₃)₄, Pd₂(dba)₃, Pd(OAc)₂; in a suitably selected organic solvent such as THF, 1,4-dioxane, toluene, and the like; to yield the corresponding compound of formula (IX).

The compound of formula (IX), is reacted with a suitably selected reducing agent such as LAH, DIBAL-H, and the like; in a suitably selected solvent such as THF, DCM, toluene, and the like; to yield the corresponding compound of formula (X).

The compound of formula (X), is reacted with a suitably substituted phenol, a compound of formula (XI), wherein A² is selected from the group consisting of C₁₋₄alkyl, preferably methyl or ethyl, a known compound or compound prepared by known methods; in the presence of a suitably selected coupling agent such as DEAD, ADDP, DIAD, and the like; in the presence of a suitably selected ligand such as triphenylphosphine (TPP), Bu₃P, and the like; in a suitably selected solvent such as DCM, THF, and the like; to yield the corresponding compound of formula (XII).

The compound of formula (XII) is reacted with a suitably selected base such as KOH, NaOH, LiOH, and the like; in a suitably selected solvent or mixture of solvents such as a mixture of THF:methanol:water, methanol, ethanol, and the like; to yield the corresponding compound of formula (I).

One skilled in the art will recognize that the compounds of formula (V) are known compounds or compounds prepared by known methods, for example as disclosed in KOLCHIN, et al., Zhumal Obshchei Khimii, 1956, pp 3731-3734, Vol. 32; and GIANTURCO, M. A., et al., Tetrahedron, 1964, pp 1763-1772, Vol. 20.

Compounds of formula (I), wherein a is 2 and b is 1 or wherein b is 2 and a is 1 (such as for example compounds of formula (I) comprising a 4-(R¹ substituted)-3,4-dihydropyranyl substituent group) may alternatively be prepared as described in Scheme 2, below.

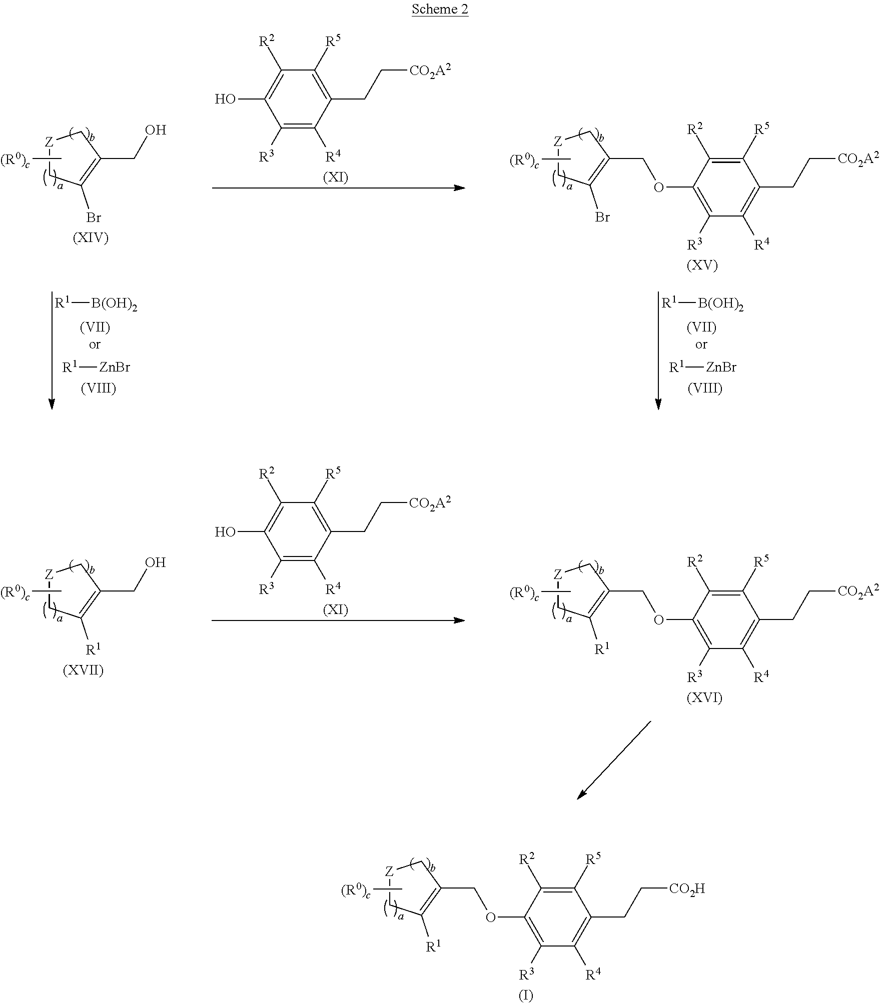

Accordingly, a suitably substituted compound of formula (XIV), a known compound or compound prepared by known methods, is reacted with a suitably substituted compound of formula (XI), wherein A² is selected from the group consisting of C₁₋₄alkyl, preferably methyl or ethyl, a known compound or compound prepared by known methods; in the presence of a suitably selected coupling agent such as DEAD, ADDP, DIAD, and the like; in the presence of a suitably selected ligand such as triphenylphosphine, Bu₃P, and the like; in a suitably selected solvent such as DCM, THF, and the like; to yield the corresponding compound of formula (XV).

The compound of formula (XV) is reacted with a suitably substituted compound of formula (VI), a known compound or compound prepared by known methods; in the presence of a suitably selected catalyst such as Pd(OAc)₂, Pd(PPh₃)₄, Pd₂(dba)₃.CH₂Cl₂, and the like; in the presence of a suitably selected ligand such as SPhos, PPh₃, dppf, and the like; in the presence of a suitably selected base such as Cs₂CO₃, K₂CO₃, K₃PO₄, and the like; in a suitably selected organic solvent such as 1,4-dioxane, toluene, DME, THF, and the like; to yield the corresponding compound of formula (XVI).

Alternatively, the compound of formula (XV) is reacted with a suitably substituted zinc bromide, a compound of formula (VIII); in the presence of a suitably selected coupling catalyst such as Pd(PPh₃)₄, Pd₂(dba)₃, Pd(OAc)₂; in a suitably selected organic solvent such as THF, 1,4-dioxane, toluene, and the like; to yield the corresponding compound of formula (XVI).

The compound of formula (XVI) is reacted with a suitably selected base such as KOH, NaOH, and the like; in a suitably selected solvent or mixture of solvents such as a mixture of THF:methanol:water, methanol, ethanol, and the like; to yield the corresponding compound of formula (I).

Alternatively, the compound of formula (XIV) is reacted with a suitably substituted compound of formula (VII), a known compound or compound prepared by known methods; in the presence of a suitably selected catalyst such as Pd(OAc)₂, Pd(PPh₃)₄, Pd₂(dba)₃.CH₂Cl₂, and the like; in the presence of a suitably selected ligand such as SPhos, PPh₃, dppf, and the like; in the presence of a suitably selected base such as Cs₂CO₃, K₂CO₃, K₃PO₄, and the like; in a suitably selected organic solvent such as 1,4-dioxane, toluene, DME, THF, and the like; to yield the corresponding compound of formula (XVII).

Alternatively, the compound of formula (XIV) is reacted with a suitably substituted zinc bromide, a compound of formula (VIII); in the presence of a suitably selected coupling catalyst such as Pd(PPh₃)₄, Pd₂(dba)₃, Pd(OAc)₂; in a suitably selected organic solvent such as THF, 1,4-dioxane, toluene, and the like; to yield the corresponding compound of formula (XVII).

The compound of formula (XVII) is reacted with a suitably substituted compound of formula (XI) wherein A² is selected from the group consisting of C₁₋₄alkyl, preferably methyl or ethyl, a known compound or compound prepared by known methods; in the presence of a suitably selected coupling agent such as DEAD, ADDP, DIAD, and the like; in the presence of a suitably selected ligand such as triphenyl phosphine, Bu₃P, and the like; in a suitably selected solvent such as DCM, THF, and the like; to yield the corresponding compound of formula (XVI).

The compound of formula (XVI) is reacted with a suitably selected base such as KOH, NaOH, and the like; in a suitably selected solvent or mixture of solvents such as a mixture of THF:methanol:water, methanol, ethanol, and the like; to yield the corresponding compound of formula (I).

Compounds of formula (XIV), wherein a is 2 and b is 1 or wherein a is 1 and b is 2 may be prepared as described in Scheme 3, below.

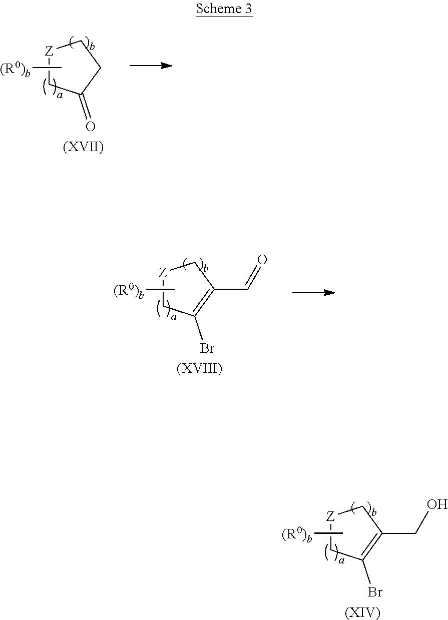

Accordingly, a suitably substituted compound of formula (XVII), a known compound or compound prepared by known methods, is reacted with a suitably selected brominating agent such as PBr₃, and the like; in the presence of DMF, neat (using DMF as the solvent); to yield the corresponding compound of formula (XVIII).

The compound of formula (XVIII) is reacted with a suitably selected reducing agent such as NaBH₄, in a solvent such as methanol, ethanol, and the like or DIBAL in a solvent such as DCM, toluene, THF, and the like; to yield the corresponding compound of formula (XIV).

Compounds of formula (I) wherein a is 1 and b is 2 (i.e. compounds of formula (I) comprising a 5-(R substituted)-3,4-dihydropyranyl substituent group) and wherein c is 0, may alternatively be prepared as described in Scheme 4, below.

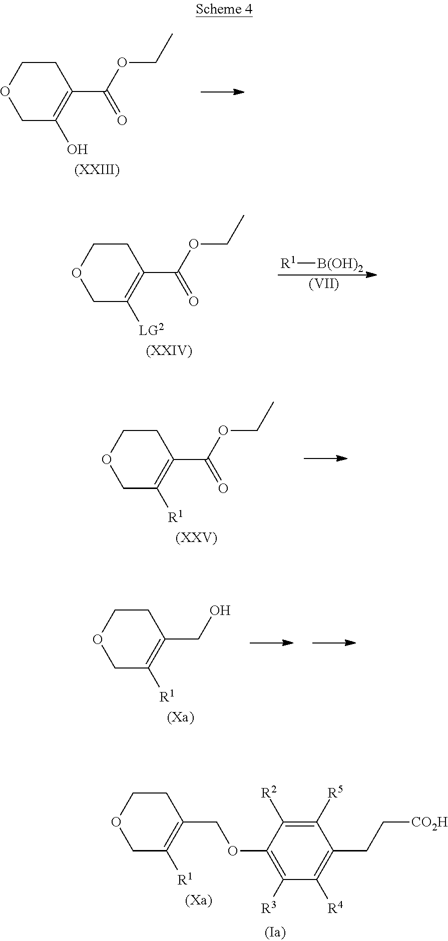

Accordingly, the compound of formula (XXIII), a known compound, is reacted with a suitably selected triflating agent such as triflic anhydride, bis(trifluoromethanesulfonimide), and the like, a known compound; in the presence of a suitably selected base such as NaH, DIPEA, TEA, and the like; in a suitably selected solvent such as THF, DCM, and the like; to yield the corresponding compound of formula (XXIV), wherein LG² is —OTf.

The compound of formula (XXIV) is reacted with a suitably substituted compound of formula (VI), a known compound or compound prepared by known methods; in the presence of a suitably selected catalyst such as Pd(dppf)Cl₂, Pd(OAc)₂, Pd(PPh₃)₄, Pd₂(dba)₃.CH₂Cl₂, and the like; in the presence of a suitably selected ligand such as SPhos, PPh₃, dppf, and the like; in the presence of a suitably selected base such as Cs₂CO₃, K₂CO₃, K₃PO₄, and the like; in a suitably selected organic solvent such as 1,4-dioxane, toluene, DME, THF, and the like; to yield the corresponding compound of formula (XXV).

The compound of formula (XXV) is reacted with a suitably selected reducing agent such as DIBAL, LiAlH₄, and the like; in a suitably selected organic solvent such as DCM, toluene, THF, and the like; at a reduced temperature of about −78° C.; to yield the corresponding compound of formula (Xa).

The compound of formula (Xa) is then substituted for the compound of formula (X), in Scheme 1, and reacted as described in Scheme 1 to yield the desired, corresponding compound of formula (Ia).

One skilled in the art will recognize that additional compounds of formula (I) wherein the

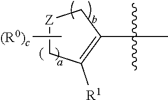

portion of the compound of formula (I) is other then

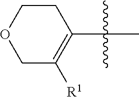

may be similarly prepared as described in Scheme 4 above, by selecting and substituting a suitably substituted compound of formula (XXI)

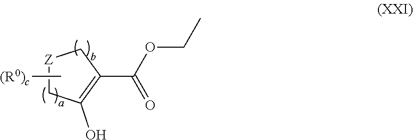

for the compound of formula (XXIII) and reacting as described in detail in Scheme 4.

One skilled in the art will recognize that compounds of formula (I) wherein X is —SO₂— may be prepared by reacting the corresponding compound of formula (I) wherein X is S with for example, oxone, in a suitably selected solvent or mixture of solvent such as a mixture of ethyl acetate and water.

### Pharmaceutical Compositions

The present invention further comprises pharmaceutical compositions containing one or more compounds of formula (I) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.01 mg to about 1000 mg or any amount or range therein, and may be given at a dosage of from about 0.01 mg/kg/day to about 300 mg/kg/day, or any amount or range therein, preferably from about 0.1 mg/kg/day to about 100 mg/kg/day, or any amount or range therein, preferably from about 0.5 mg/kg/day to about 50 mg/kg/day, preferably from about 1.0 mg/kg/day to about 25 mg/kg/day, or any amount or range therein. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from about 0.01 mg to about 1,000 mg, or any amount or range therein, of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method(s) of treating disorders as described herein may also be carried out using a pharmaceutical composition comprising any of the compounds as defined herein and a pharmaceutically acceptable carrier. The pharmaceutical composition may contain between about 0.01 mg and about 1000 mg of the compound, or any amount or range therein; preferably from about 1.0 mg to about 500 mg of the compound, or any amount or range therein, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixers, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methylcellulose and the like. For parenteral administration, sterile suspensions and solutions are desired. Isotonic preparations which generally contain suitable preservatives are employed when intravenous administration is desired.

To prepare a pharmaceutical composition of the present invention, a compound of formula (I) as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets. Second Edition. Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of this invention may be administered in any of the foregoing compositions and according to dosage regimens established in the art whenever treatment of disorders, as described herein, is required.

The daily dosage of the products may be varied over a wide range from about 0.01 mg to about 1,000 mg per adult human per day, or any amount or range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the patient to be treated. An effective amount of the drug is ordinarily supplied at a dosage level of from about 0.01 mg/kg to about 300 mg/kg of body weight per day, or any amount or range therein. Preferably, the range is from about 0.1 to about 100.0 mg/kg of body weight per day, or any amount or range therein. More preferably, from about 0.5 to about 50.0 mg/kg of body weight per day, or any amount or range therein. More preferably, from about 1.0 to about 25.0 mg/kg of body weight per day, or any amount or range therein. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

### SYNTHESIS EXAMPLES

The Examples are set forth to aid in the understanding of the invention, and are not intended and should not be construed to limit in any way the invention set forth in the claims which follow thereafter.

In the Examples which follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

### Example 1

### 3-[4-[[5-(4-chlorophenyl)-2,3,6,7-tetrahydrooxepin-4-yl]methoxy]-2,3-dimethyl-phenyl]propanoic acid

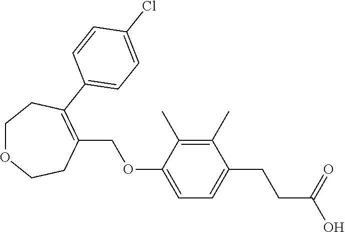 [SMILES: Cc1c(CCC(=O)O)ccc(OCC2=C(c3ccc(Cl)cc3)CCOCC2)c1C]

### Step 1. Ethyl 5-oxooxepane-4-carboxylate

Into a 50-mL round-bottom flask purged and maintained with an inert atmosphere of nitrogen, was placed a solution of oxan-4-one (500 mg, 4.99 mmol, 1.00 equiv) in dichloromethane (30 mL), ethyl diazoethanoate (1.00 mL, 1.50 equiv). This was followed by the addition of BF₃.Et₂O (625 mg, 4.40 mmol, 0.88 equiv) dropwise with stirring at −78° C. The resulting solution was stirred for 30 min at −78° C. in a dry ice bath. The reaction was then quenched by the addition of aqueous sodium carbonate (50 mL). The resulting solution was extracted with DCM (3×50 mL) and the organic layers combined and dried over anhydrous sodium sulfate. The resulting mixture was concentrated under vacuum and the residue was applied onto a silica gel column with ethyl acetate/petroleum ether (1:15) to yield ethyl 5-oxooxepane-4-carboxylate as light yellow oil.

### Step 2. Ethyl 5-[(trifluoromethane)sulfonyloxy]-2,3,6,7-tetrahydrooxepine-4-carboxylate

Into a 100-mL round-bottom flask, was placed a solution of ethyl 5-oxooxepane-4-carboxylate (700 mg, 3.76 mmol, 1.00 equiv) in diethyl ether (30 mL). Sodium hydride (181 mg, 7.54 mmol, 2.01 equiv) was added at 0° C. The resulting solution was stirred for 2 h at 20° C. This was followed by the addition of Tf₂O (1.60 g, 5.67 mmol, 1.51 equiv) at 0° C. The resulting solution was stirred for 2 h at 20° C. The reaction was then quenched by the addition of ice water (50 mL). The resulting solution was extracted with ethyl acetate (3×50 mL) and the organic layers combined. The resulting mixture was washed with aqueous sodium chloride (3×50 mL) and dried over anhydrous sodium sulfate. The resulting mixture was concentrated under vacuum to yield ethyl 5-[(trifluoromethane)sulfonyloxy]-2,3,6,7-tetrahydrooxepine-4-carboxylate as a yellow oil.

### Step 3. Ethyl 5-(4-chlorophenyl)-2,3,6,7-tetrahydrooxepine-4-carboxylate

Into a 100-mL round-bottom flask purged and maintained with an inert atmosphere of nitrogen, was placed a solution of ethyl 5-[(trifluoromethane)sulfonyloxy]-2,3,6,7-tetrahydrooxepine-4-carboxylate (500 mg, 1.57 mmol, 1.00 equiv) in 1,4-dioxane (30 mL), (4-chlorophenyl)boronic acid (245 mg, 1.57 mmol, 1.00 equiv), Pd(PPh₃)₄ (91 mg, 0.080 mmol, 0.05 equiv), K₃PO₄ (667 mg, 3.14 mmol, 2.00 equiv). The resulting solution was stirred for 16 h at 80° C. in an oil bath. The resulting mixture was concentrated under vacuum. The residue was applied onto a silica gel column with ethyl acetate/petroleum ether (1:5) to yield ethyl 5-(4-chlorophenyl)-2,3,6,7-tetrahydrooxepine-4-carboxylate as yellow oil.

### Step 4. [5-(4-chlorophenyl)-2,3,6,7-tetrahydrooxepin-4-yl]methanol

Into a 50-mL 3-necked round-bottom flask purged and maintained with an inert atmosphere of nitrogen, was added a solution of ethyl 5-(4-chlorophenyl)-2,3,6,7-tetrahydrooxepine-4-carboxylate (300 mg, 1.07 mmol, 1.00 equiv) in dichloromethane (20 mL). This was followed by the addition of DIBAL (2.1 mL) dropwise with stirring at −78° C. The resulting solution was stirred for 2 h at −78° C. in a dry ice bath. The reaction was then quenched by the addition of aqueous NH₄Cl (30 mL). The resulting solution was extracted with DCM (3×100 mL) and the organic layers combined and dried over anhydrous sodium sulfate. The residue was applied onto a silica gel column with ethyl acetate/petroleum ether (1:1) to yield [5-(4-chlorophenyl)-2,3,6,7-tetrahydrooxepin-4-yl]methanol as light yellow oil.

### Step 5. Ethyl 3-(4-[[5-(4-chlorophenyl)-2,3,6,7-tetrahydrooxepin-4-yl]methoxy]-2,3-dimethylphenyl) propanoate

Into a 50-mL 3-necked round-bottom flask purged and maintained with an inert atmosphere of nitrogen, was added a solution of [5-(4-chlorophenyl)-2,3,6,7-tetrahydrooxepin-4-yl]methanol (100 mg, 0.42 mmol, 1.00 equiv) in dichloromethane (20 mL), ethyl 3-(4-hydroxy-2,3-dimethylphenyl)propanoate (93 mg, 0.42 mmol, 1.00 equiv), PPh₃ (220 mg, 0.840 mmol, 2.00 equiv). The resulting solution was stirred for 10 min at 0° C. DIAD (170 mg, 0.84 mmol, 2.01 equiv) was then added. The resulting solution was stirred for 16 h at 25° C. The resulting mixture was concentrated under vacuum. The residue was applied onto a silica gel column with ethyl acetate/petroleum ether (1:10) to yield ethyl 3-(4-[[5-(4-chlorophenyl)-2,3,6,7-tetrahydrooxepin-4-yl]methoxy]-2,3-dimethylphenyl)propanoate as light yellow oil.

### Step 6. 3-(4-[[5-(4-chlorophenyl)-2,3,6,7-tetrahydrooxepin-4-yl]methoxy]-2,3-dimethylphenyl)propanoic acid

Into a 50-mL round-bottom flask, was added a solution of ethyl 3-(4-[[5-(4-chlorophenyl)-2,3,6,7-tetrahydrooxepin-4-yl]methoxy]-2,3-dimethylphenyl)propanoate (120 mg, 0.27 mmol, 1.0 equiv) in tetrahydrofuran (10 mL), LiOH (120 mg, 5.01 mmol, 18.5 equiv), water (5 mL). The resulting solution was stirred for 16 h at 25° C. The pH value of the solution was adjusted to 2 with hydrogen chloride (2N). The resulting solution was extracted with ethyl acetate (3×50 mL) and the organic layers combined. The resulting mixture was washed with aqueous sodium chloride (3×50 mL). The mixture was dried over anhydrous sodium sulfate. The solids were filtered out. The resulting mixture was concentrated under vacuum. The residue was washed by CH₃CN and dried by vacuum to yield 3-(4-[[5-(4-chlorophenyl)-2,3,6,7-tetrahydrooxepin-4-yl]methoxy]-2,3-dimethylphenyl)propanoic acid as a white solid.

¹H NMR (300 MHz, CDCl₃) δ: 7.27 (d, J=8.4 Hz, 2H), 7.07 (d, J=8.4 Hz, 2H), 6.87 (d, J=8.4 Hz, 1H), 6.38 (d, J=8.4 Hz, 1H), 4.26 (s, 2H), 3.76-3.80 (m, 4H), 2.88-2.93 (m, 2H), 2.66-2.75 (m, 4H), 2.53-2.59 (m, 2H), 2.21 (s, 3H), 2.16 (s, 3H). Mass spectrum (ESI, m/z): Calculated for C₂₄H₂₇ClO₄, 413.2 [M−H], Measured 413.1.

The following compound was similarly prepared according to the procedure as described in Example 1 above, selecting and substituting suitably substituted reactants, as would be readily recognized by those skilled in the art.

### Example 2

### 3-[4-[[5-(4-chlorophenyl)-2,3,6,7-tetrahydrooxepin-4-yl]methoxy]-2-(trifluoromethyl)phenyl]propanoic acid

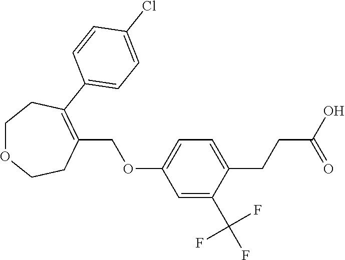 [SMILES: O=C(O)CCc1ccc(OCC2=C(c3ccc(Cl)cc3)CCOCC2)cc1C(F)(F)F]

¹H NMR (300 MHz, CD₃OD) δ: 7.28-7.39 (m, 3H), 7.13-7.17 (m, 2H), 6.97 (s, 1H), 6.86-6.91 (m, 1H), 4.41 (s, 2H), 3.76 (t, J=4.8 Hz, 2H), 3.69 (t, J=4.8 Hz, 2H), 2.99 (t, J=7.5 Hz, 2H), 2.74 (t, J=4.8 Hz, 2H), 2.65 (t, J=4.8 Hz, 2H), 2.58 (t, J=7.2 Hz, 2H). ¹⁹F NMR (300 MHz, CD₃OD) δ: −61.26. Mass spectrum (ESI, m/z): Calculated for C₂₃H₂₂ClF₃O₄, 453.1 [M−H], Measured 453.1.

### Example 3

### 3-[4-[[5-(p-tolyl)-2,3,6,7-tetrahydrooxepin-4-yl]methoxy]-2-(trifluoromethyl)phenyl]propanoic acid

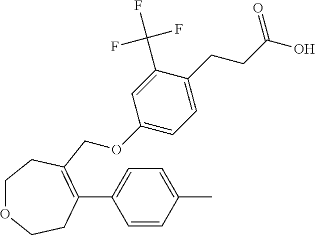 [SMILES: Cc1ccc(C2=C(COc3ccc(CCC(=O)O)c(C(F)(F)F)c3)CCOCC2)cc1]

### Step 1. Ethyl 5-(4-methylphenyl)-2,3,6,7-tetrahydrooxepine-4-carboxylate

Into a 100-mL round-bottom flask purged and maintained with an inert atmosphere of nitrogen, was added a solution of ethyl 5-[(trifluoromethane)sulfonyloxy]-2,3,6,7-tetrahydrooxepine-4-carboxylate (500 mg, 1.570 mmol, 1.00 equiv) in 1,4-dioxane (30 ml), (4-methylphenyl)boronic acid (257 mg, 1.890 mmol, 1.20 equiv), Pd(PPh₃)₄ (91 mg, 0.080 mmol, 0.05 equiv), and K₃PO₄ (667 mg, 3.140 mmol, 2.00 equiv). The resulting solution was stirred for 16 h at 90° C. in an oil bath. The resulting mixture was concentrated under vacuum and the residue was applied onto a silica gel column with ethyl acetate/petroleum ether (1:5) to yield ethyl 5-(4-methylphenyl)-2,3,6,7-tetrahydrooxepine-4-carboxylate as colorless oil.

### Step 2. [5-(4-methylphenyl)-2,3,6,7-tetrahydrooxepin-4-yl]methanol

Into a 50-mL 3-necked round-bottom flask purged and maintained with an inert atmosphere of nitrogen was added a solution of ethyl 5-(4-methylphenyl)-2,3,6,7-tetrahydrooxepine-4-carboxylate (320 mg, 1.230 mmol, 1.00 equiv) in dichloromethane (15 mL). The resulting solution was stirred at −78° C. and DIBAL (2.5 mL) was then added dropwise at this temperature. The mixture was warmed to 20° C. gradually and stirred for 2 h. The resulting mixture was quenched by methanol and concentrated under vacuum. The residue was applied onto a silica gel column with ethyl acetate/petroleum ether (1:3) to yield [5-(4-methylphenyl)-2,3,6,7-tetrahydrooxepin-4-yl]methanol as colorless oil.

### Step 3. Ethyl 3-(4-[[5-(4-methylphenyl)-2,3,6,7-tetrahydrooxepin-4-yl]methoxy]-2-(trifluoromethyl) phenyl) propanoate

Into a 50-mL 3-necked round-bottom flask purged and maintained with an inert atmosphere of nitrogen was added a solution of [5-(4-methylphenyl)-2,3,6,7-tetrahydrooxepin-4-yl]methanol (80 mg, 0.370 mmol, 1.00 equiv) in dichloromethane (10 mL), ethyl 3-[4-hydroxy-2-(trifluoromethyl)phenyl]propanoate (98 mg, 0.370 mmol, 1.02 equiv), and PPh₃ (192 mg, 0.730 mmol, 2.00 equiv). The mixture was cooled to 0° C. and stirred for 10 min. DIAD (146 mg, 0.720 mmol, 1.97 equiv) was then added dropwise. The resulting solution was stirred for 16 h at 20° C. The resulting mixture was quenched with aqueous NH₄Cl (20 mL). The resulting solution was extracted with ethyl acetate (3×50 mL) and the organic layers combined. The resulting mixture was washed with aqueous sodium chloride (3×50 mL). The mixture was dried over anhydrous sodium sulfate, concentrated under vacuum and the residue was applied onto a silica gel column with ethyl acetate/petroleum ether (1:6) to yield ethyl 3-(4-[[5-(4-methylphenyl)-2,3,6,7-tetrahydrooxepin-4-yl]methoxy]-2-(trifluoromethyl)phenyl)propanoate as colorless oil.

### Step 4. 3-(4-[[5-(4-methylphenyl)-2,3,6,7-tetrahydrooxepin-4-yl]methoxy]-2-(trifluoromethyl)phenyl) propanoic acid

Into a 50-mL round-bottom flask was added a solution of ethyl 3-(4-[[5-(4-methylphenyl)-2,3,6,7-tetrahydrooxepin-4-yl]methoxy]-2-(trifluoromethyl)phenyl)propanoate (80 mg, 0.170 mmol, 1.00 equiv) in tetrahydrofuran/H₂O (5 ml, 3:2), and lithium hydroxide (80 mg, 3.340 mmol, 19.31 equiv). The resulting solution was stirred for 16 h at 20° C. The pH value of the solution was adjusted to 3 with hydrogen chloride (2N). The resulting solution was extracted with ethyl acetate (3×50 mL) and the organic layers combined. The resulting mixture was washed with aqueous sodium chloride (3×50 mL) and dried over anhydrous sodium sulfate. The resulting mixture was concentrated under vacuum and the residue was purified by reversed-phase HPLC on a SunFire C18 column (19 mm×150 mm, 5 μM) with a linear gradient of 50-90% acetonitrile in water (0.05% TFA) in 10 mins to yield 3-(4-[[5-(4-methylphenyl)-2,3,6,7-tetrahydrooxepin-4-yl]methoxy]-2-(trifluoromethyl)phenyl)propanoic acid as an off-white solid.

¹H NMR (300 MHz, CD₃OD) δ: 7.30 (d, J=8.4 Hz, 1H), 7.16 (d, J=8.1 Hz, 2H), 7.04 (d, J=8.1 Hz, 2H), 6.92 (s, 1H), 6.83-6.87 (m, 1H), 4.42 (s, 2H), 3.75 (t, J=4.8 Hz, 2H), 3.67 (t, J=4.8 Hz, 2H), 2.98 (t, J=7.5 Hz, 2H), 2.74 (t, J=4.8 Hz, 2H), 2.64 (t, J=4.8 Hz, 2H), 2.41 (t, J=7.8 Hz, 2H), 2.33 (s, 3H). ¹⁹F NMR (300 MHz, CD₃OD) δ: −61.17. Mass spectrum (ESI, m/z): Calculated. for C₂₄H₂₅F₃O₄, 433.2 [M−H], Measured 433.1.

The following compounds were similarly prepared according to the procedure as described in Example 3 above, selecting and substituting suitably substituted reactants, as would be readily recognized by those skilled in the art.

### Example 4

### 3-[4-[(5-cyclopentyl-2,3,6,7-tetrahydrooxepin-4-yl)methoxy]phenyl]propanoic acid

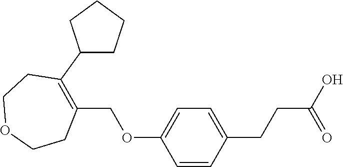 [SMILES: O=C(O)CCc1ccc(OCC2=C(C3CCCC3)CCOCC2)cc1]

¹H NMR (CHLOROFORM-d) δ: 7.12 (d, J=8.6 Hz, 2H), 6.85 (d, J=8.6 Hz, 2H), 4.51 (s, 2H), 3.61 (td, J=4.7, 1.3 Hz, 4H), 2.97-3.10 (m, 1H), 2.85-2.95 (m, 2H), 2.61-2.69 (m, 2H), 2.48-2.54 (m, 2H), 2.29-2.40 (m, 2H), 1.55-1.72 (m, 6H), 1.27-1.39 (m, 2H). Mass spectrum (ESI, m/z): Calculated. for C₂₁H₂₈O₄, 367.1, [M+Na], Measured 367.3.

### Example 5

### 3-[4-[(5-cyclopentyl-2,3,6,7-tetrahydrooxepin-4-yl)methoxy]-2-(trifluoromethyl)phenyl]propanoic acid

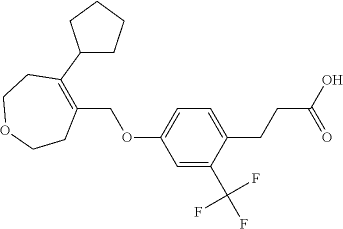 [SMILES: O=C(O)CCc1ccc(OCC2=C(C3CCCC3)CCOCC2)cc1C(F)(F)F]

¹H NMR (CHLOROFORM-d) δ: 7.23-7.29 (m, 1H), 7.17 (d, J=2.5 Hz, 1H), 7.01 (dd, J=8.6, 2.5 Hz, 1H), 4.55 (s, 2H), 3.57-3.67 (m, 4H), 2.94-3.14 (m, 3H), 2.60-2.70 (m, 2H), 2.46-2.54 (m, 2H), 2.30-2.40 (m, 2H), 1.55-1.71 (m, 6H), 1.28-1.41 (m, 2H). Mass spectrum (ESI, m/z): Calculated for C₂₃H₂₇F₃O₄, 435.1, [M+Na], Measured 435.3.

### Example 6

### 3-[4-[[5-(3-fluoro-4-methyl-phenyl)-2,3,6,7-tetrahydrooxepin-4-yl]methoxy]phenyl]propanoic acid

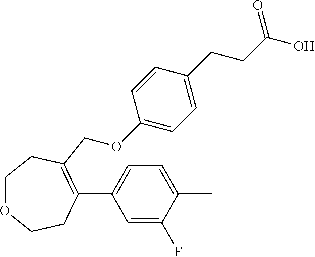 [SMILES: Cc1ccc(C2=C(COc3ccc(CCC(=O)O)cc3)CCOCC2)cc1F]

¹H NMR (CHLOROFORM-d) δ: 7.08-7.17 (m, 1H), 7.06 (d, J=8.6 Hz, 2H), 6.75-6.84 (m, 2H), 6.63-6.73 (m, 2H), 4.32 (s, 2H), 3.68-3.82 (m, 4H), 2.82-2.94 (m, 2H), 2.70-2.77 (m, 2H), 2.55-2.69 (m, 4H), 2.25 (d, J=1.5 Hz, 3H). Mass spectrum (ESI, m/z): Calculated for C₂₃H₂₅FO₄, 407.1, [M+Na], Measured 407.3.

### Example 7

### 3-[2-chloro-4-[(5-cyclopentyl-2,3,6,7-tetrahydrooxepin-4-yl)methoxy]phenyl]propanoic acid

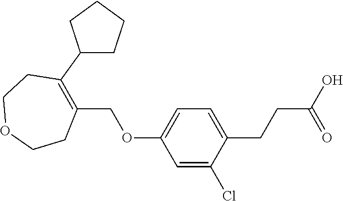 [SMILES: O=C(O)CCc1ccc(OCC2=C(C3CCCC3)CCOCC2)cc1Cl]

¹H NMR (CHLOROFORM-d) δ: 7.15 (d, J=8.6 Hz, 1H), 6.93 (d, J=2.5 Hz, 1H), 6.76 (dd, J=8.1, 2.5 Hz, 1H), 4.51 (s, 2H), 3.52-3.66 (m, 4H), 3.00 (br t, J=7.8 Hz, 3H), 2.66 (t, J=7.8 Hz, 2H), 2.44-2.53 (m, 2H), 2.31-2.39 (m, 2H), 1.50-1.76 (m, 6H), 1.21-1.40 (m, 2H). Mass spectrum (ESI, m/z): Calculated for C₂₁H₂₇ClO₄, 401.1, [M+Na], Measured 401.3.

### Example 8

### 3-[3,5-difluoro-4-[[5-(3-fluoro-4-methyl-phenyl)-2,3,6,7-tetrahydrooxepin-4-yl]methoxy]phenyl]propanoic acid

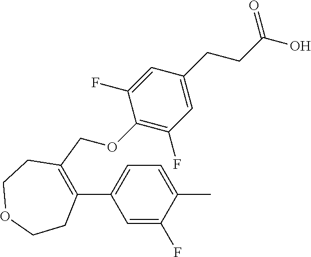 [SMILES: Cc1ccc(C2=C(COc3c(F)cc(CCC(=O)O)cc3F)CCOCC2)cc1F]

¹H NMR (CHLOROFORM-d) δ: 7.09 (t, J=7.8 Hz, 1H), 6.64-6.83 (m, 4H), 4.37 (s, 2H), 3.79-3.84 (m, 2H), 3.74-3.79 (m, 2H), 2.83-2.91 (m, 2H), 2.74-2.79 (m, 2H), 2.68-2.73 (m, 2H), 2.61-2.68 (m, 2H), 2.25 (d, J=2.0 Hz, 3H). Mass spectrum (ESI, m/z): Calculated for C₂₃H₂₃F₃O₄, 443.1, [M+Na], Measured 443.3.

### Example 9

### 3-[4-[[5-(3-fluoro-4-methyl-phenyl-2,3,6,7-tetrahydrooxepin-4-yl]methoxy]-2,3-dimethyl-phenyl]propanoic acid

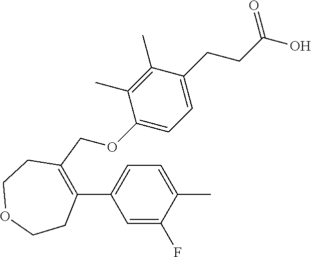 [SMILES: Cc1ccc(C2=C(COc3ccc(CCC(=O)O)c(C)c3C)CCOCC2)cc1F]

¹H NMR (CHLOROFORM-d) δ: 7.10 (t, J=7.8 Hz, 1H), 6.87 (d, J=8.1 Hz, 1H), 6.72-6.84 (m, 2H), 6.40 (d, J=8.1 Hz, 1H), 4.30 (s, 2H), 3.66-3.84 (m, 4H), 2.84-2.95 (m, 2H), 2.70-2.77 (m, 2H), 2.64-2.70 (m, 2H), 2.49-2.61 (m, 2H), 2.25 (d, J=1.5 Hz, 3H), 2.21 (s, 3H), 2.16 (s, 3H). Mass spectrum (ESI, m/z): Calculated for C₂₅H₂₉FO₄, 435.1, [M−H], Measured 435.3.

### Example 10

### 3-[5-fluoro-4-[[5-(3-fluoro-4-methyl-phenyl)-2,3,6,7-tetrahydrooxepin-4-yl]methoxy]-2-methyl-phenyl]propanoic acid

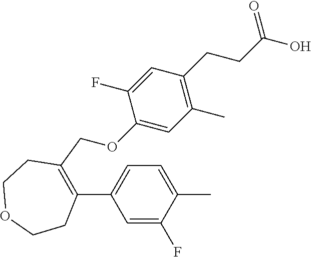 [SMILES: Cc1ccc(C2=C(COc3cc(C)c(CCC(=O)O)cc3F)CCOCC2)cc1F]

10 ¹H NMR (CHLOROFORM-d) δ: 7.13 (t, J=7.8 Hz, 1H), 6.74-6.87 (m, 3H), 6.46 (d, J=8.1 Hz, 1H), 4.40 (s, 2H), 3.67-3.84 (m, 4H), 2.79-2.88 (m, 2H), 2.70 (dt, J=16.4, 4.7 Hz, 4H), 2.54-2.63 (m, 2H), 2.27 (d, J=1.5 Hz, 3H), 2.18 (s, 3H). Mass spectrum (ESI, m/z): Calculated for C₂₄H₂₆F₂O₄, 439.1, [M+Na], Measured 439.3.

### Example 11

### 3-[2,3-difluoro-4-[[5-(3-fluoro-4-methyl-phenyl-2,3,6,7-tetrahydrooxepin-4-yl]methoxy]phenyl]propanoic acid

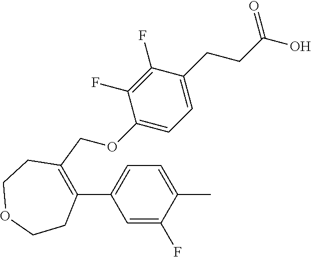 [SMILES: Cc1ccc(C2=C(COc3ccc(CCC(=O)O)c(F)c3F)CCOCC2)cc1F]

¹H NMR (CHLOROFORM-d) δ: 7.13 (t, J=7.8 Hz, 1H), 6.72-6.83 (m, 3H), 6.35-6.45 (m, 1H), 4.40 (s, 2H), 3.75 (dt, J=9.6, 4.8 Hz, 4H), 2.87-2.98 (m, 2H), 2.70-2.76 (m, 2H), 2.60-2.70 (m, 5H), 2.26 (d, J=1.5 Hz, 3H).). Mass spectrum (ESI, m/z): Calculated for C₂₃H₂₃F₃O₄, 443.1, [M+Na], Measured 443.3.

### Example 12

### 3-[4-[[5-(3-fluoro-4-methyl-phenyl)-2,3,6,7-tetrahydrooxepin-4-yl]methoxy]-2-(trifluoromethyl)phenyl]propanoic acid

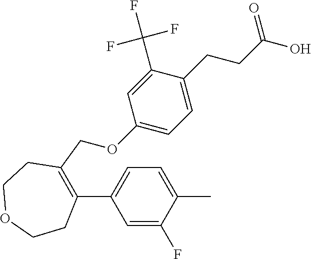 [SMILES: Cc1ccc(C2=C(COc3ccc(CCC(=O)O)c(C(F)(F)F)c3)CCOCC2)cc1F]

¹H NMR (CHLOROFORM-d) δ: 7.20 (d, J=8.6 Hz, 1H), 7.13 (t, J=7.8 Hz, 1H), 7.00 (d, J=2.5 Hz, 1H), 6.85 (dd, J=8.6, 2.5 Hz, 1H), 6.75-6.82 (m, 2H), 4.37 (s, 2H), 3.69-3.81 (m, 4H), 3.04 (t, J=7.8 Hz, 2H), 2.71-2.77 (m, 2H), 2.58-2.67 (m, 4H), 2.26 (d, J=2.0 Hz, 3H). Mass spectrum (ESI, m/z): Calculated for C₂₄H₂₄F₄O₄, 475.1, [M+Na], Measured 475.3.

### Example 13

### 3-[4-[[5-(4-chlorophenyl)-3,6-dihydro-2H-pyran-4-yl]methoxy]-2,3-dimethyl-phenyl]propanoic acid

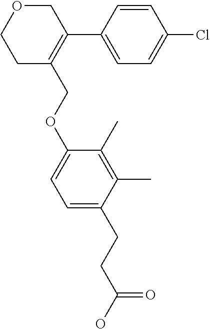 [SMILES: Cc1c(CCC(=O)O)ccc(OCC2=C(c3ccc(Cl)cc3)COCC2)c1C]

### Step A: ethyl 5-(((trifluoromethyl)sulfonyl)oxy)-3,6-dihydro-2H-pyran-4-carboxylate

To a solution of ethyl 3-oxotetrahydro-2H-pyran-4-carboxylate (4.40 g, 25.50 mmol) and DIEA (13.21 ml, 76.66 mmol) in 120 ml of DCM at 0° C. was added triflic anhydride (4.62 ml, 28.11 mmol) and the mixture was allowed to stir overnight at room temperature. DCM was removed under vacuum and the residue dissolved in EtOAc and washed with 1N HCl and then brine. The mixture was dried over anhydrous sodium sulfate and concentrated under vacuum. The residue was purified on a silica gel column eluting with 20% EtOAc/heptane to yield ethyl 5-(((trifluoromethyl)sulfonyl)oxy)-3,6-dihydro-2H-pyran-4-carboxylate as a light brown oil. Mass spectrum (ESI, m/z): Calculated for C₉H₁₁F₃O₆S, 305.2 (M+H), Measured 305.0.

### Step B: ethyl 5-(4-chlorophenyl)-3,6-dihydro-2H-pyran-4-carboxylate

To a solution of ethyl 5-(((trifluoromethyl)sulfonyl)oxy)-3,6-dihydro-2H-pyran-4-carboxylate (4.97 g, 16.34 mmol) in 109 ml of 25% water/1,4-dioxane was added 4-chlorophenylboronic acid (3.83 g, 24.50 mmol), Pd(dppf)Cl₂ (668 mg, 0.82 mmol), and potassium phosphate (13.87 g, 65.34 mmol) and the mixture was heated at 80° C. for 40 mins. The mixture was diluted with EtOAc and washed with water and brine, then dried over anhydrous sodium sulfate and concentrated under vacuum. The residue was purified on a silica gel column eluting with 20% EtOAc/heptane to yield ethyl 5-(4-chlorophenyl)-3,6-dihydro-2H-pyran-4-carboxylate as a colorless oil. Mass spectrum (ESI, m/z): Calculated for C₁₄H₁₅ClO₃, 267.7 (M+H), Measured 267.0.

### Step C: (5-(4-chlorophenyl)-3,6-dihydro-2H-pyran-4-yl)methanol

To a solution of ethyl 5-(4-chlorophenyl)-3,6-dihydro-2H-pyran-4-carboxylate (4.00 g, 15.00 mmol) in 25 mL of toluene at −40° C. was added diisobutylaluminum hydride (1M in toluene, 37.49 ml, 37.49 mmol) and the mixture was allowed to stir for 1 hr at −40° C. The reaction was quenched with sodium sulfate decahydrate and stirred for 1 hr at room temperature. To the mixture was then added diethyl ether (25 ml) and the mixture was filtered and concentrated. The residue was purified on a silica gel column eluting with 7-70% EtOAc/heptane step gradient to yield (5-(4-chlorophenyl)-3,6-dihydro-2H-pyran-4-yl)methanol as a colorless oil. Mass spectrum (ESI, m/z): Calculated for C₁₂H₁₃ClO₂, 249.7 (M+Na), Measured 249.0.

### Step D: ethyl 3-(4-((5-(4-chlorophenyl)-3,6-dihydro-2H-pyran-4-yl)methoxy)-2,3-dimethylphenyl)propanoate

To a solution of (5-(4-chlorophenyl)-3,6-dihydro-2H-pyran-4-yl)methanol (2.80 g, 12.46 mmol), ethyl 3-(4-hydroxy-2,3-dimethylphenyl)propanoate (2.77 g, 12.46 mmol) and 1,1′-(azodicarbonyl)dipiperidine (4.86 g, 18.69 mmol) in 62 ml of THF at room temperature was added tri-n-butylphosphine (4.76 mL, 18.69 mmol) and the mixture heated to 60° C. for 90 mins. The mixture was concentrated and then added 1:1 DCM/heptane to the residue (approx. 10 ml) and filtered the resulting precipitate. The filtrate was loaded on to a silica gel column and eluted with 20% EtOAc/hep to yield ethyl 3-(4-((5-(4-chlorophenyl)-3,6-dihydro-2H-pyran-4-yl)methoxy)-2,3-dimethylphenyl)propanoate as a colorless oil. Mass spectrum (ESI, m/z): Calculated for C₂₅H₂₉ClO₄, 451.9 (M+Na), Measured 451.3.

### STEP E: 3-(4-((5-(4-chlorophenyl)-3,6-dihydro-2H-pyran-4-yl)methoxy)-2,3-dimethylphenyl)propanoic acid

To a solution of ethyl 3-(4-((5-(4-chlorophenyl)-3,6-dihydro-2H-pyran-4-yl)methoxy)-2,3-dimethylphenyl)propanoate (4.48 g, 10.44 mmol) in 26 ml of 10% MeOH/THF was added 3M NaOH (6.96 mL, 20.89 mmol) and the mixture stirred for 8 hrs at room temperature. The mixture was then diluted with water (20 ml) and acidified to pH 2 with 3N HCl and concentrated to remove organics. A precipitate formed that was collected by filtration and washed with water and dried to yield 3-(4-((5-(4-chlorophenyl)-3,6-dihydro-2H-pyran-4-yl)methoxy)-2,3-dimethylphenyl)propanoic acid as a white solid.

¹H NMR (400 MHz, CHLOROFORM-d) δ: 7.30 (d, J=8.59 Hz, 2H), 7.13 (d, J=8.59 Hz, 2H), 6.89 (d, J=8.59 Hz, 1H), 6.44 (d, J=8.59 Hz, 1H), 4.31 (s, 2H), 4.28 (s, 2H), 3.95 (t, J=5.56 Hz, 2H), 2.85-2.97 (m, 2H), 2.53-2.62 (m, 2H), 2.43 (dt, J=5.31, 2.91 Hz, 2H), 2.22 (s, 3H), 2.17 (s, 3H). Mass spectrum (ESI, m/z): Calculated for C₂₃H₂₅ClO₄, 422.9 (M+Na), Measured 423.0.

The following compounds were similarly prepared according to the procedure as described in Example 13 above, selecting and substituting suitably substituted reactants, as would be readily recognized by those skilled in the art.

### Example 14

### 3-[4-[[5-(4-chlorophenyl)-3,6-dihydro-2H-pyran-4-yl]methoxy]-3,5-difluoro-phenyl]propanoic acid

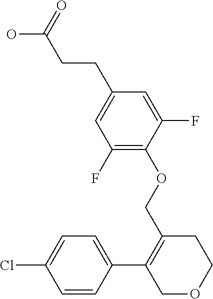

¹H NMR (400 MHz, CD₃OD) δ: 7.30 (d, J=8.0 Hz, 2H), 6.94 (d, J=8.6 Hz, 2H), 6.82 (d, J=8.6 Hz, 2H), 4.41 (s, 2H), 4.21 (s, 2H), 3.92 (t, J=7.8 Hz, 2H), 2.88 (t, J=7.8 Hz, 2H), 2.52 (t, J=7.8 Hz, 2H), 1.45 (s, 2H). ¹⁹F NMR (400 MHz, CD₃OD) δ: −129.81. Mass spectrum (ESI, m/z): Calculated for C₂₁H₁₉ClF₂O₄, 407.1 [M−H], Measured 407.0.

### Example 15

### 3-[4-[[5-(4-chlorophenyl)-3,6-dihydro-2H-pyran-4-yl]methoxy]-3-methyl-phenyl]propanoic acid

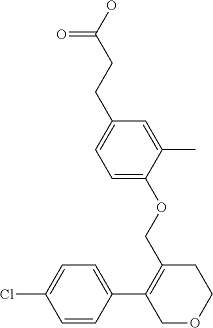 [SMILES: Cc1cc(CCC(=O)O)ccc1OCC1=C(c2ccc(Cl)cc2)COCC1]

¹H NMR (300 MHz, CD₃OD) δ: 7.31 (d, J=8.1 Hz, 2H), 7.17 (d, J=8.7 Hz, 2H), 6.94 (s, 1H), 6.84-6.87 (m, 1H), 6.47 (d, J=8.4 Hz, 1H), 4.32 (s, 2H), 4.24 (s, 2H), 3.88 (t, J=5.7 Hz, 2H), 2.77 (t, J=7.5 Hz, 2H), 2.50 (t, J=7.5 Hz, 2H), 2.36-2.39 (m, 2H), 2.14 (s, 3H). Mass spectrum (ESI, m/z): Calculated for C₂₂H₂₃ClO₄, 385.1 [M−H], Measured 385.1.

### Example 16

### 3-[4-[[5-(4-chlorophenyl)-3,6-dihydro-2H-pyran-4-yl]methoxy]-2-(trifluoromethyl)phenyl]propanoic acid

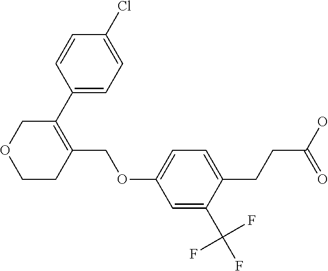 [SMILES: O=C(O)CCc1ccc(OCC2=C(c3ccc(Cl)cc3)COCC2)cc1C(F)(F)F]

¹H NMR (300 MHz, CD₃OD) δ: 7.26-7.40 (m, 3H), 7.15-7.23 (m, 2H), 7.00 (s, 1H), 6.89-6.93 (m, 1H), 4.41 (s, 2H), 4.24 (s, 2H), 3.87 (t, J=5.4 Hz, 2H), 2.96-3.01 (m, 2H), 2.50-2.57 (m, 2H), 2.32-2.36 (m, 2H). ¹⁹F NMR (300 MHz, CD₃OD) δ: −61.21, −77.59. Mass spectrum (ESI, m/z): Calculated for C₂₂H₂₀ClF₃O₄, 439.1 [M−H], Measured 439.0.

### Example 17

### 3-[4-[[5-(4-chlorophenyl)-3,6-dihydro-2H-pyran-4-yl]methoxy]-2-(cyclopropylmethyl)phenyl]propanoic acid

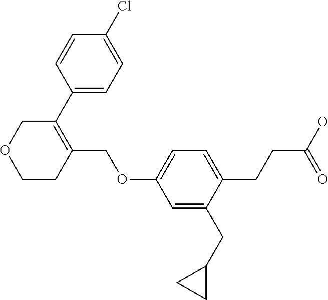 [SMILES: O=C(O)CCc1ccc(OCC2=C(c3ccc(Cl)cc3)COCC2)cc1CC1CC1]

¹H NMR (300 MHz, CD₃OD) δ: 7.36 (d, J=6.6 Hz, 2H), 7.20 (d, J=8.4 Hz, 2H), 7.02 (d, J=8.1 Hz, 1H), 6.72 (s, 1H), 6.53-6.57 (m, 1H), 4.37 (s, 2H), 4.27 (s, 2H), 3.90 (t, J=5.7 Hz, 2H), 2.86 (t, J=7.5 Hz, 2H), 2.47-2.51 (m, 4H), 2.37-2.41 (m, 2H), 0.88-0.94 (m, 1H), 0.47-0.53 (m, 2H), 0.13-0.18 (m, 2H). Mass spectrum (ESI, m/z): Calculated for C₂₅H₂₇ClO₄, 425.2 [M−H], Measured 425.1.

### Example 18

### 3-[4-[[5-(D-tolyl)-3,6-dihydro-2H-pyran-4-yl]methoxy]-2-(trifluoromethyl)phenyl]propanoic acid

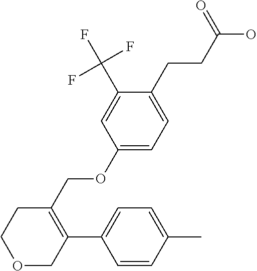

¹H NMR (300 MHz, CD₃OD) δ: 7.30 (d, J=8.7 Hz, 1H), 7.19 (d, J=8.4 Hz, 2H), 7.09 (d, J=8.4 Hz, 2H), 7.00 (s, 1H), 6.93 (d, J=8.7 Hz, 1H), 4.47 (s, 2H), 4.27 (s, 2H), 3.90 (t, J=5.4 Hz, 2H), 3.00 (t, J=7.5 Hz, 2H), 2.54 (t, J=8.4 Hz, 2H), 2.34-2.37 (m, 5H). ¹⁹F NMR (300 MHz, CD₃OD) δ: −61.32. Mass spectrum (ESI, m/z): Calculated for C₂₃H₂₃F₃O₄, 419.2 [M−H], Measured 419.1.

### Example 19

### 3-[4-[[5-(4-methyl-2-phenyl)-phenyl)-3,6-dihydro-2H-pyran-4-yl]methoxy]-2-(trifluoromethyl)phenyl]propanoic acid

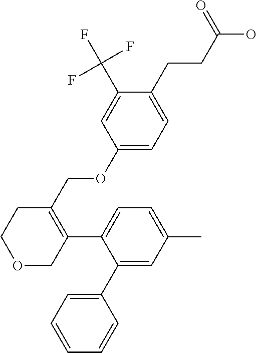

¹H NMR (300 MHz, CD₃OD) δ: 7.29-7.42 (m, 6H), 7.12-7.22 (m, 3H), 6.95 (s, 1H), 6.88 (d, J=8.4 Hz, 1H), 4.28-4.32 (m, 1H), 4.15-4.20 (m, 1H), 3.85-3.99 (m, 2H), 3.70-3.83 (m, 2H), 3.01 (t, J=7.8 Hz, 2H), 2.55 (t, J=7.8 Hz, 2H), 2.41 (s, 3H), 2.20-2.25 (m, 1H), 2.06-2.14 (m, 1H). ¹⁹F NMR (300 MHz, CD₃OD) δ: −61.31, −77.04. Mass spectrum (ESI, m/z): Calculated For C₃₀H₂₇F₄O, 495.2 [M-1], Measured 495.1.

### Example 20

### 3-[4-[[5-(4-methyl-3-phenyl-phenyl)-3,6-dihydro-2H-pyran-4-yl]methoxy]-2-(trifluoromethyl)phenyl]propanoic acid

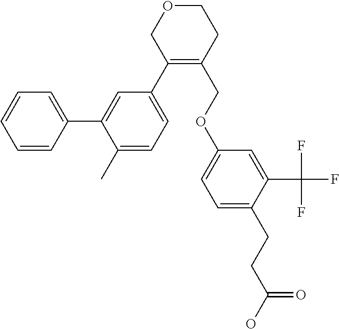

¹H NMR (300 MHz, CD₃OD) δ: 7.29-7.37 (m, 5H), 7.12-7.21 (m, 3H), 7.05-7.07 (m, 2H), 6.96-7.00 (m, 1H), 4.52 (5, 2H), 4.35 (5, 2H), 3.93 (t, J=5.4 Hz, 2H), 3.00-3.06 (m, 2H), 2.57 (t, J=8.4 Hz, 2H), 2.39-2.42 (m, 2H), 2.25 (5, 3H). ¹⁹F NMR (300 MHz, CD₃OD) δ: −61.28, −76.96. Mass spectrum (ESI, m/z): Calculated for C₂₉H₂₇F₄O₄, 495.2 [M−H], Measured 495.1.

### Example 21

### 3-[4-[[5-(2-fluoro-4-methyl-phenyl)-3,6-dihydro-2H-pyran-4-yl]methoxy]-2-(trifluoromethyl)phenyl]propanoic acid

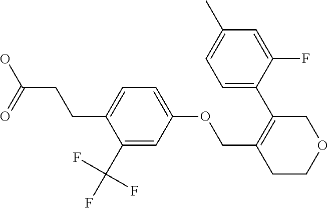

¹H NMR (300 MHz, CD₃OD) δ: 7.31 (d, J=8.1 Hz, 1H), 6.92-7.11 (m, 5H), 4.43 (s, 2H), 4.23 (s, 2H), 3.92 (t, J=5.7 Hz, 2H), 3.01 (t, J=7.8 Hz, 2H), 2.55 (t, J=8.4 Hz, 2H), 2.37-2.39 (m, 5H). ¹⁹F NMR (300 MHz, CD₃OD) δ: −62.89, −79.12, −119.38. Mass spectrum (ESI, m/z): Calculated for C₂₃H₂₂F₄O₄, 437.1 [M−H], Measured 436.9.

### Example 22

### 3-[4-[[5-(2,4-dimethylphenyl)-3,6-dihydro-2H-pyran-4-yl]methoxy]-2-(trifluoromethyl)phenyl]propanoic acid

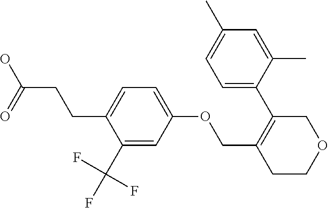

¹H NMR (300 MHz, CD₃OD) δ: 7.31 (d, J=8.7 Hz, 1H), 6.81-7.05 (m, 5H), 4.22-4.31 (m, 2H), 4.13 (s, 2H), 3.92 (t, J=7.8 Hz, 2H), 2.99 (t, J=7.8 Hz, 2H), 2.27-2.44 (m, 7H), 2.19 (s, 3H). ¹⁹F NMR (300 MHz, CD₃OD) δ: −61.26. Mass spectrum (ESI, m/z): Calculated for C₂₄H₂₅F₃O₄, 433.2 [M−H], Measured 433.1.

### Example 23

### 3-[4-[[5-(3-isopropyl-4-methyl-phenyl)-3,6-dihydro-2H-pyran-4-yl]methoxy]-2-(trifluoromethyl)phenyl]propanoic acid

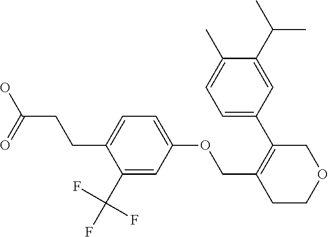

¹H NMR (300 MHz, CD₃OD) δ: 7.30 (d, J=8.4 Hz, 1H), 7.04-7.14 (m, 3H), 6.90-6.97 (m, 2H), 4.43 (s, 2H), 4.30 (s, 2H), 3.91 (t, J=5.4 Hz, 2H), 3.07-3.16 (m, 1H), 3.09 (t, J=6.9 Hz, 2H), 2.53 (t, J=7.8 Hz, 2H), 2.38-2.40 (m, 2H), 2.31 (s, 3H), 1.09 (d, J=6.9 Hz, 6H). ¹⁹F NMR (300 MHz, CD₃OD) δ: −61.33. Mass spectrum (ESI, m/z): Calculated for C₂₆H₂₉F₃O₄, 461.2 [M−H], Measured 461.0.

### Example 24

### 3-[4-[[5-(2-isopropyl-4-methyl-phenyl)-3,6-dihydro-2H-pyran-4-yl]methoxy]-2-(trifluoromethyl)phenyl]propanoic acid

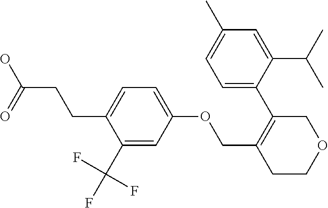

¹H NMR (300 MHz, CD₃OD) δ: 7.31 (d, J=8.7 Hz, 1H), 7.16 (s, 1H), 3.88-7.00 (m, 4H), 4.26 (s, 2H), 4.02-4.26 (m, 2H), 3.89-3.99 (m, 2H), 2.96-3.10 (m, 3H), 2.45-2.55 (m, 3H), 2.33-2.36 (m, 4H), 1.21-1.30 (m, 6H). ¹⁹F NMR (300 MHz, CD₃OD) δ: −61.28. Mass spectrum (ESI, m/z): Calculated for C₂₆H₂₉F₃O₄, 461.2 [M−H], Measured 461.1.

### Example 25

### 3-[4-[(5-phenyl-3,6-dihydro-2H-pyran-4-yl)methoxy]-2-(trifluoromethyl)phenyl]propanoic acid

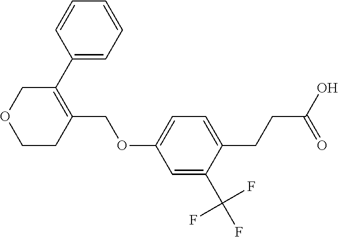 [SMILES: O=C(O)CCc1ccc(OCC2=C(c3ccccc3)COCC2)cc1C(F)(F)F]

¹H NMR (300 MHz, CD₃OD) δ: 7.30-7.40 (m, 4H), 7.20-7.26 (m, 2H), 7.01 (s, 1H), 6.93 (d, J=8.4 Hz, 1H), 4.46 (s, 2H), 4.29 (s, 2H), 3.91 (t, J=5.4 Hz, 2H), 2.99 (t, J=7.8 Hz, 2H), 2.53 (t, J=7.8 Hz, 2H), 2.35-2.39 (m, 2H). ¹⁹F NMR (300 MHz, CD₃OD) δ: −61.32, −77.48. Mass spectrum (ESI, m/z): Calculated for C₂₂H₂₁F₃O₄, 405.1 [M−H], Measured 405.1.

### Example 26

### 3-[4-[(5-cyclopentyl-3,6-dihydro-2H-pyran-4-yl)methoxy]phenyl]propanoic acid

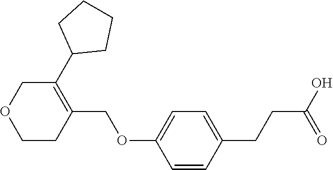 [SMILES: O=C(O)CCc1ccc(OCC2=C(C3CCCC3)COCC2)cc1]

¹H NMR (CHLOROFORM-d) δ: 7.13 (d, J=8.6 Hz, 2H), 6.87 (d, J=8.6 Hz, 2H), 4.49 (s, 2H), 4.13 (s, 2H), 3.82 (t, J=5.8 Hz, 2H), 2.91 (t, J=7.8 Hz, 3H), 2.55-2.73 (m, 2H), 2.27 (dt, J=5.3, 2.9 Hz, 2H), 1.45-1.81 (m, 6H), 1.28-1.45 (m, 2H). Mass spectrum (ESI, m/z): Calculated for C₂₀H₂₆O₄, 353.1 [M+Na], Measured 353.2.

### Example 27

### 3-[4-[(5-cyclopentyl-3,6-dihydro-2H-pyran-4-yl)methoxy]-2-(trifluoromethyl)phenyl]propanoic acid

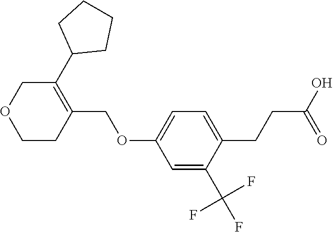 [SMILES: O=C(O)CCc1ccc(OCC2=C(C3CCCC3)COCC2)cc1C(F)(F)F]

¹H NMR (CHLOROFORM-d) δ: 7.26-7.30 (m, 2H), 7.19 (d, J=2.5 Hz, 1H), 7.03 (dd, J=8.1, 2.5 Hz, 1H), 4.54 (s, 2H), 4.14 (s, 2H), 3.81 (t, J=5.8 Hz, 2H), 3.08 (br t, J=7.8 Hz, 2H), 2.87-3.00 (m, 1H), 2.65 (t, J=7.8 Hz, 2H), 2.25 (dt, J=5.3, 2.9 Hz, 2H), 1.52-1.79 (m, 6H), 1.33-1.46 (m, 2H). Mass spectrum (ESI, m/z): Calculated for C₂₁H₂₅F₃O₄, 421.1 [M+Na], Measured 421.2.

### Example 28

### 3-[4-[[5-(3-fluoro-4-methyl-phenyl)-3,6-dihydro-2H-pyran-4-yl]methoxy]-2,3-dimethyl-phenyl]propanoic acid

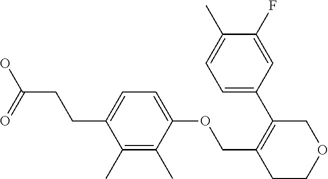

¹H NMR (CHLOROFORM-d) δ: 7.13 (s, 1H), 6.81-6.91 (m, 3H), 6.46 (d, J=8.6 Hz, 1H), 4.31-4.39 (m, 2H), 4.27 (s, 2H), 3.94 (t, J=5.6 Hz, 2H), 2.87-2.97 (m, 2H), 2.53-2.62 (m, 2H), 2.42 (dt, J=5.2, 2.7 Hz, 2H), 2.26 (d, J=1.5 Hz, 3H), 2.21 (s, 3H), 2.18 (s, 3H). Mass spectrum (ESI, m/z): Calculated for C₂₄H₂₇FO₄, 421.1 (M+Na), Measured 421.3.

### Example 29

### 3-[3,5-difluoro-4-[[5-(3-fluoro-4-methyl-phenyl)-3,6-dihydro-2H-pyran-4-yl]methoxy]phenyl]propanoic acid

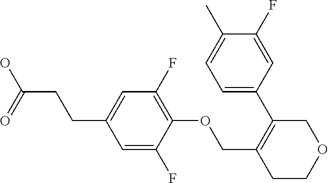

¹H NMR (CHLOROFORM-d) δ: 7.10 (t, J=7.8 Hz, 1H), 6.68-6.82 (m, 3H), 6.63 (dd, J=10.6, 1.5 Hz, 1H), 4.40 (s, 2H), 4.23 (s, 2H), 3.94 (t, J=5.6 Hz, 2H), 2.82-2.95 (m, 2H), 2.61-2.72 (m, 2H), 2.46-2.58 (m, 2H), 2.18-2.33 (m, 3H). Mass spectrum (ESI, m/z): Calculated for C₂₂H₂₁F₃O₄, 429.4 (M+Na), Measured 429.0.

### Example 30

### 3-[2,3-difluoro-4-[[5-(3-fluoro-4-methyl-phenyl)-3,6-dihydro-2H-pyran-4-yl]methoxy]phenyl]propanoic acid

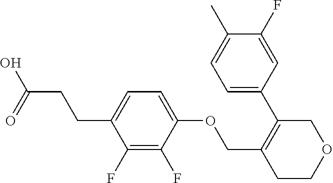 [SMILES: Cc1ccc(C2=C(COc3ccc(CCC(=O)O)c(F)c3F)CCOC2)cc1F]

¹H NMR (CHLOROFORM-d) δ: 7.15 (t, J=8.1 Hz, 1H), 6.71-6.91 (m, 3H), 6.38-6.51 (m, 1H), 4.43 (s, 2H), 4.26 (s, 2H), 3.93 (t, J=5.6 Hz, 2H), 2.92 (t, J=7.6 Hz, 2H), 2.60-2.73 (m, 2H), 2.41 (dt, J=5.3, 2.9 Hz, 2H), 2.27 (d, J=1.5 Hz, 3H). Mass spectrum (ESI, m/z): Calculated for C₂₂H₂₁F₃O₄, 429.4 (M+Na), Measured 429.0.

### Example 31

### 3-[4-[[5-(3-fluoro-4-methyl-phenyl-3,6-dihydro-2H-pyran-4-yl]methoxy]phenyl]propanoic acid

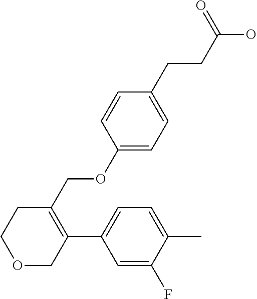

¹H NMR (CHLOROFORM-d) δ: 7.14 (t, J=7.8 Hz, 1H), 7.08 (d, J=8.6 Hz, 2H), 6.82-6.89 (m, 2H), 6.73 (d, J=8.6 Hz, 2H), 4.33-4.40 (m, 2H), 4.22-4.30 (m, 2H), 3.93 (t, J=5.6 Hz, 1H), 2.85-2.93 (m, 2H), 2.60-2.68 (m, 2H), 2.39 (dt, J=5.3, 2.9 Hz, 2H), 2.26 (d, J=1.5 Hz, 3H). Mass spectrum (ESI, m/z): Calculated for C₂₂H₂₃FO₄, 393.1 [M+Na], Measured 393.3.

### Example 32

### 3-[6-[[5-(3-fluoro-4-methyl-phenyl)-3,6-dihydro-2H-pyran-4-yl]methoxy]-2-methyl-3-pyridyl]propanoic acid

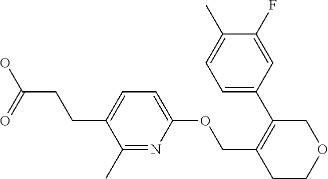

¹H NMR (CHLOROFORM-d) δ: 7.95 (d, J=8.6 Hz, 1H), 7.18 (t, J=7.6 Hz, 1H), 6.78-6.88 (m, 2H), 6.66 (d, J=9.1 Hz, 1H), 4.67 (s, 2H), 4.28 (s, 2H), 3.92 (t, J=5.6 Hz, 2H), 2.94 (t, J=6.8 Hz, 2H), 2.59-2.73 (m, 5H), 2.35 (br s, 2H), 2.27 (d, J=1.5 Hz, 3H). Mass spectrum (ESI, m/z): Calculated for C₂₂H₂₄FNO₄, 386.1 [M+H], Measured 386.3.

### Example 33

### 3-[4-[[4-(4-chlorophenyl)-3,6-dihydro-2H-pyran-5-yl]methoxy]-2,3-dimethyl-phenyl]propanoic acid

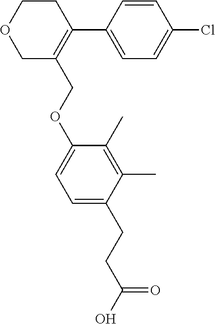 [SMILES: Cc1c(CCC(=O)O)ccc(OCC2=C(c3ccc(Cl)cc3)CCOC2)c1C]

### Step 1: Methyl 4-oxooxane-3-carboxylate

To a solution of dimethyl carbonate (22.50 g, 249.78 mmol, 5.00 equiv) in tetrahydrofuran (70 mL) at 0° C. was added sodium hydride (6.00 g, 150.00 mmol, 3.00 equiv, 60%). The mixture was stirred at 15° C. for 1 h and then the mixture was cooled to 0° C. and oxan-4-one (5.00 g, 49.940 mmol, 1.00 equiv) was added dropwise. The resulting solution was stirred for 2 h at 60° C. in an oil bath. The reaction was then quenched by the addition of water and the pH adjusted to 6 with hydrogen chloride (2N). The resulting solution was extracted with ethyl acetate (3×20 mL) and the organic layers combined and concentrated under vacuum. The residue was applied onto a silica gel column with ethyl acetate/petroleum ether (10/90). The collected fractions were combined and concentrated under vacuum to yield methyl 4-oxooxane-3-carboxylate as yellow oil.

### Step 2. Methyl 4-[(trifluoromethane)sulfonyloxy]-5,6-dihydro-2H-pyran-3-carboxylate

To a solution of methyl 4-oxooxane-3-carboxylate (550 mg, 3.480 mmol, 1.00 equiv) in diethyl ether (20 mL) at 0° C. was added sodium hydride (208 mg, 5.200 mmol, 1.50 equiv, 60%) and the mixture was stirred at 15° C. for 1 h. Triflic anhydride (1.50 g, 5.320 mmol, 1.53 equiv) was then added and resulting mixture was stirred overnight at 15° C. The reaction was quenched by the addition of water and the resulting solution was extracted with ethyl acetate (3×10 mL) and the organic layers combined and dried over anhydrous sodium sulfate and concentrated under vacuum to yield methyl 4-[(trifluoromethane)sulfonyloxy]-5,6-dihydro-2H-pyran-3-carboxylate as yellow oil.

### Step 3. Methyl 4-(4-chlorophenyl)-5,6-dihydro-2H-pyran-3-carboxylate

Into a 100-mL round-bottom flask purged and maintained with an inert atmosphere of nitrogen was added methyl 4-[(trifluoromethane)sulfonyloxy]-5,6-dihydro-2H-pyran-3-carboxylate (1.33 g, 4.580 mmol, 1.00 equiv), (4-chlorophenyl)boronic acid (2.86 g, 18.290 mmol, 3.99 equiv), Pd(PPh₃)₄ (530 mg, 0.460 mmol, 0.10 equiv), K₃PO₄ (3.80 g, 17.900 mmol, 3.91 equiv) and 1,4-dioxane (20 mL). The resulting solution was stirred overnight at 90° C. in an oil bath. The resulting mixture was concentrated under vacuum. The residue was applied onto a silica gel column with ethyl acetate/petroleum ether (20/80). The collected fractions were combined and concentrated under vacuum to yield methyl 4-(4-chlorophenyl)-5,6-dihydro-2H-pyran-3-carboxylate as a white solid.

### Step 4. [4-(4-chlorophenyl)-5,6-dihydro-2H-pyran-3-yl]methanol

To a solution of methyl 4-(4-chlorophenyl)-5,6-dihydro-2H-pyran-3-carboxylate (150 mg, 0.590 mmol, 1.00 equiv) in toluene (10 mL) at −70° C. was added DIBAL (1N, 1.78 mL) dropwise with stirring. The resulting solution was stirred for 2 h at 15° C. and then quenched by the addition of NH₄Cl/H₂O. The pH was adjusted to 6 with hydrogen chloride (2N) and the mixture was extracted with ethyl acetate (3×10 mL) and the organic layers combined and dried over anhydrous sodium sulfate. The residue was applied onto a silica gel column with ethyl acetate/petroleum ether (57/43) to yield [4-(4-chlorophenyl)-5,6-dihydro-2H-pyran-3-yl]methanol as colorless oil.

### Step 5. Ethyl 3-(4-[[4-(4-chlorophenyl)-5,6-dihydro-2H-pyran-3-yl]methoxy]-2,3-dimethylphenyl) propanoate

Into a 50-mL round-bottom flask purged and maintained with an inert atmosphere of nitrogen was added [4-(4-chlorophenyl)-5,6-dihydro-2H-pyran-3-yl]methanol (110 mg, 0.490 mmol, 1.00 equiv), ethyl 3-(4-hydroxy-2,3-dimethylphenyl)propanoate (130 mg, 0.580 mmol, 1.19 equiv), ADDP (312 mg, 1.250 mmol, 2.55 equiv), Bu₃P (151 mg, 0.750 mmol, 1.53 equiv) and toluene (4 mL). The resulting mixture was stirred overnight at 60° C. and then concentrated under vacuum. The residue was applied onto a silica gel column with ethyl acetate/petroleum ether (1/3). The collected fractions were combined and concentrated under vacuum to yield ethyl 3-(4-[[4-(4-chlorophenyl)-5,6-dihydro-2H-pyran-3-yl]methoxy]-2,3-dimethylphenyl)propanoate as colorless oil.

### Step 6. [[4-(4-chlorophenyl)-5,6-dihydro-2H-pyran-3-yl]methoxy]-2,3-dimethylphenyl)propanoic acid

Into a 50-mL round-bottom flask was added ethyl 3-(4-[[4-(4-chlorophenyl)-5,6-dihydro-2H-pyran-3-yl]methoxy]-2,3-dimethylphenyl)propanoate (95 mg, 0.220 mmol, 1.00 equiv), LiOH (95 mg, 3.970 mmol, 17.91 equiv), tetrahydrofuran (2 mL) and water (2 mL). The resulting solution was stirred overnight at 15° C. The pH was adjusted to 5 with hydrogen chloride (2N) and the resulting mixture was extracted with ethyl acetate (3×2 mL) and the organic layers combined and concentrated under vacuum. The residue was purified by reversed-phase HPLC to yield 3-(4-[[4-(4-chlorophenyl)-5,6-dihydro-2H-pyran-3-yl]methoxy]-2,3-dimethylphenyl)propanoic acid as a white solid.

¹H NMR (300 MHz, CDCl₃) δ: 7.30 (d, J=8.4 Hz, 2H), 7.16 (d, J=8.4 Hz, 2H), 6.88 (d, J=8.1 Hz, 1H), 6.43 (d, J=8.1 Hz, 1H), 4.39 (s, 2H), 4.31 (s, 2H), 3.94 (t, J=5.4 Hz, 2H), 2.91 (t, J=7.5 Hz, 2H), 2.57 (t, J=8.4 Hz, 2H), 2.40-2.45 (m, 2H), 2.21 (s, 3H), 2.01 (s, 3H). Mass spectrum (ESI, m/z): Calculated for C₂₃H₂₅ClO₄, 418.1 [M+NH₃], Measured 418.1.

The following compounds were similarly prepared according to the procedure as described in Example 33 above, selecting and substituting suitably substituted reactants, as would be readily recognized by those skilled in the art.

### Example 34

### 3-[4-[[4-(4-chlorophenyl)-3,6-dihydro-2H-pyran-5-yl]methoxy]-3,5-difluoro-phenyl]propanoic acid

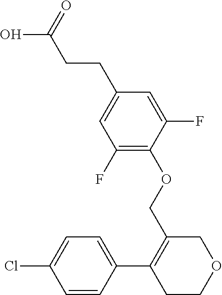 [SMILES: O=C(O)CCc1cc(F)c(OCC2=C(c3ccc(Cl)cc3)CCOC2)c(F)c1]

¹H NMR (300 MHz, CD₃OD) δ: 7.31 (d, J=8.4 Hz, 2H), 7.05 (d, J=9.0 Hz, 2H), 6.83 (d, J=9.6 Hz, 2H), 4.39-4.44 (m, 4H), 3.89 (t, J=5.4 Hz, 2H), 2.84 (t, J=8.1 Hz, 2H), 2.61 (t, J=8.1 Hz, 2H), 2.38-2.42 (m, 2H). ¹⁹F NMR (300 MHz, CD3OD) δ: −129.8. Mass spectrum (ESI, m/z): Calculated for C₂₁H₁₉ClF₂O₄, 407.1 [M−H], Measured 406.9.

### Example 35

### 3-[4-[[4-(4-chlorophenyl)-3,6-dihydro-2H-pyran-5-yl]methoxy]-3-methyl-phenyl]propanoic acid

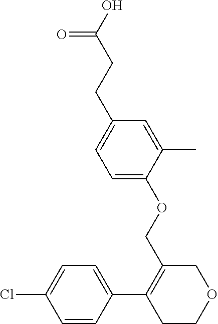 [SMILES: Cc1cc(CCC(=O)O)ccc1OCC1=C(c2ccc(Cl)cc2)CCOC1]

¹H NMR (300 MHz, CD₃OD) δ: 7.36 (d, J=8.1 Hz, 2H), 7.24 (d, J=8.1 Hz, 2H), 6.78 (s, 1H), 6.88-6.91 (m, 1H), 6.54 (d, J=8.1 Hz, 1H), 4.26 (s, 4H), 3.91 (t, J=5.7 Hz, 2H), 2.79 (t, J=7.5 Hz, 2H), 2.46-2.56 (m, 4H), 2.17 (s, 3H). Mass spectrum (ESI, m/z): Calculated for C₂₂H₂₃ClO₄, 385.1 [M−H], Measured 385.1.

### Example 36

### 3-[4-[[4-(4-chlorophenyl)-3,6-dihydro-2H-pyran-5-yl]methoxy]-2-(trifluoromethyl)phenyl]propanoic acid

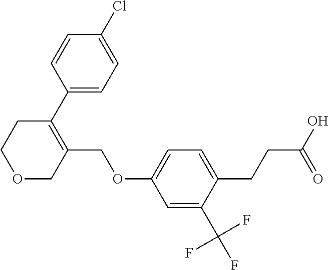 [SMILES: O=C(O)CCc1ccc(OCC2=C(c3ccc(Cl)cc3)CCOC2)cc1C(F)(F)F]

¹H NMR (300 MHz, CD₃OD) δ: 7.35-7.38 (m, 3H), 7.23 (d, J=6.3 Hz, 2H), 7.03 (s, 1H), 6.89-6.99 (m, 1H), 4.44 (s, 2H), 4.32 (s, 2H), 3.91 (t, J=5.7 Hz, 2H), 2.98-3.00 (m, 2H), 2.46-2.55 (m, 4H). Mass spectrum (ESI, m/z): Calculated for C₂₂H₂₀ClF₃O₄, 439.1 [M−H], Measured 439.0.

### Example 37

### 3-[4-[[4-(4-methoxyphenyl)-3,6-dihydro-2H-pyran-5-yl]methoxy]-2,3-dimethyl-phenyl]propanoic acid

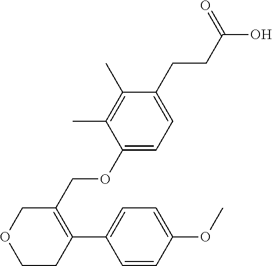 [SMILES: COc1ccc(C2=C(COc3ccc(CCC(=O)O)c(C)c3C)COCC2)cc1]

¹H NMR (300 MHz, CD₃OD) δ: 7.16 (d, J=8.7 Hz, 2H), 6.91-6.85 (m, 3H), 6.43 (d, J=8.4 Hz, 1H), 4.35-4.37 (m, 4H), 3.93 (t, J=7.6 Hz, 2H), 3.79 (s, 3H), 2.87 (t, J=7.6 Hz, 2H), 2.51-2.46 (m, 4H), 2.22 (s, 3H), 2.16 (s, 3H). Mass spectrum (ESI, m/z): Calculated for C₂₄H₂₈O₅, 395.2 [M−H], Measured 395.1.

### Example 38

### 3-[4-[[4-(4-fluorophenyl)-3,6-dihydro-2H-pyran-5-yl]methoxy]-2,3-dimethyl-phenyl]propanoic acid

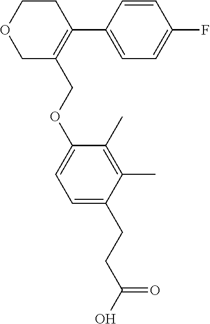 [SMILES: Cc1c(CCC(=O)O)ccc(OCC2=C(c3ccc(F)cc3)CCOC2)c1C]

¹H NMR (300 MHz, CD₃OD) δ: 7.22-7.28 (m, 2H), 7.03-7.11 (m, 2H), 6.86 (d, J=8.4 Hz, 1H), 6.43 (d, J=8.4 Hz, 1H), 4.36 (s, 2H), 4.34 (s, 2H), 3.92 (t, J=7.6 Hz, 2H), 2.87 (t, J=7.6 Hz, 2H), 2.44-2.50 (m, 4H), 2.21 (s, 3H), 2.14 (s, 3H). ¹⁹F NMR (300 MHz, CD₃OD) δ: −116.97. Mass spectrum (ESI, m/z): Calculated for C₂₃H₂₅FO₄, 383.2 [M−H], Measured 383.1.

### Example 39

### 3-[4-[[4-(2-fluorophenyl)-3,6-dihydro-2H-pyran-5-yl]methoxy]-2,3-dimethyl-phenyl]propanoic acid

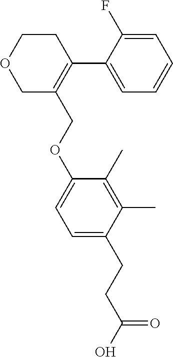 [SMILES: Cc1c(CCC(=O)O)ccc(OCC2=C(c3ccccc3F)CCOC2)c1C]

¹H NMR (300 MHz, CD₃OD) δ: 7.30-7.37 (m, 1H), 7.09-7.29 (m, 3H), 6.83 (d, J=8.7 Hz, 1H), 6.40 (d, J=8.7 Hz, 1H), 4.39 (s, 2H), 4.30 (s, 2H), 3.91 (t, J=5.4 Hz, 2H), 2.85 (t, J=7.6 Hz, 2H), 2.42-2.48 (m, 4H), 2.19 (s, 3H), 2.11 (s, 3H). ¹⁹F NMR (300 MHz, CD₃OD) δ: −117.48. Mass spectrum (ESI, m/z): Calculated for C₂₃H₂₅FO₄, 383.2 [M−H], Measured 383.1.

### Example 40

### 3-[2,3-dimethyl-4-[[4-(3-pyridyl)-3,6-dihydro-2H-pyran-5-yl]methoxy]phenyl]propanoic acid

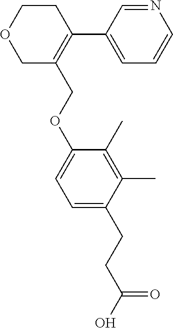 [SMILES: Cc1c(CCC(=O)O)ccc(OCC2=C(c3cccnc3)CCOC2)c1C]

¹H NMR (300 MHz, CD₃OD) δ: 8.72 (s, 2H), 8.37 (d, J=9.6 Hz, 1H), 7.94-7.99 (m, 1H), 6.88 (d, J=8.7 Hz, 1H), 6.50 (d, J=8.7 Hz, 1H), 4.41 (s, 2H), 4.36 (s, 2H), 3.97 (t, J=7.6 Hz, 2H), 2.86 (t, J=7.6 Hz, 2H), 2.44-2.54 (m, 4H), 2.23 (s, 3H), 2.01 (s, 3H). Mass spectrum (ESI, m/z): Calculated for C₂₂H₂₅NO₄, 368.2 [M+H], Measured 368.1.

### Example 41

### 3-[2,3-dimethyl-4-[[4-(p-tolyl)-3,6-dihydro-2H-pyran-5-yl]methoxy]phenyl]propanoic acid

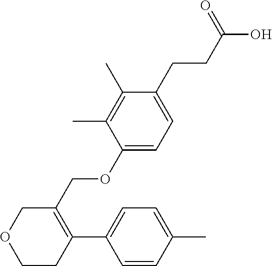 [SMILES: Cc1ccc(C2=C(COc3ccc(CCC(=O)O)c(C)c3C)COCC2)cc1]

¹H NMR (300 MHz, CD₃OD) δ: 7.09-7.12 (m, 4H), 6.82 (d, J=8.4 Hz, 1H), 6.40 (d, J=8.4 Hz, 1H), 4.35 (s, 4H), 3.90 (t, J=7.6 Hz, 2H), 2.86 (t, J=7.6 Hz, 2H), 2.45-2.49 (m, 4H), 2.33 (s, 3H), 2.20 (s, 3H), 2.14 (s, 3H). Mass spectrum (ESI, m/z): Calculated for C₂₄H₂₈O₄, 379.2 [M−H], Measured 379.1.

### Example 42

### 3-[4-[[4-(3-methoxyphenyl)-3,6-dihydro-2H-pyran-5-yl]methoxy]-2,3-dimethyl-phenyl]propanoic acid

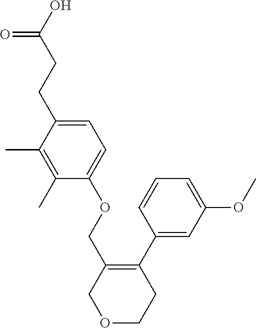

¹H NMR (300 MHz, CD₃OD) δ: 7.23-7.28 (m, 1H), 6.79-6.87 (m, 4H), 6.44 (d, J=8.4 Hz, 1H), 4.37 (s, 4H), 3.94 (t, J=7.6 Hz, 2H), 3.67 (s, 3H), 2.87 (t, J=7.6 Hz, 2H), 2.45-2.50 (m, 4H), 2.21 (s, 3H), 2.16 (s, 3H). Mass spectrum (ESI, m/z): Calculated for C₂₄H₂₈O₅, 395.2 [M−H], Measured 395.1.

### Example 43

### 3-[4-[[4-(3,4-difluorophenyl)-3,6-dihydro-2H-pyran-5-yl]methoxy]-2,3-dimethyl-phenyl]propanoic acid

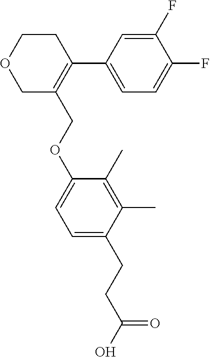 [SMILES: Cc1c(CCC(=O)O)ccc(OCC2=C(c3ccc(F)c(F)c3)CCOC2)c1C]

¹H NMR (300 MHz, CD₃OD) δ: 7.17-7.25 (m, 1H), 6.83-7.01 (m, 3H), 6.41 (d, J=8.4 Hz, 1H), 4.38 (s, 2H), 4.28 (s, 2H), 3.90 (t, J=5.4 Hz, 2H), 2.86 (t, J=7.5 Hz, 2H), 2.31-2.45 (m, 4H), 2.19 (s, 3H), 2.10 (s, 3H). ¹⁹F NMR (300 MHz, CD₃OD) δ: −112.75, −112.90. Mass spectrum (ESI, m/z): Calculated for C₂₃H₂₄F₂O₄, 401.2 [M−H], Measured 401.2.

### Example 44

### 3-[4-[[4-(3-fluoro-4-methyl-phenyl-3,6-dihydro-2H-pyran-5-yl]methoxy]-2,3-dimethyl-phenyl]propanoic acid

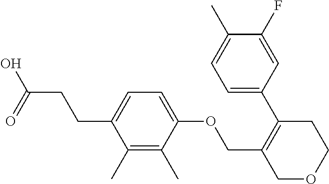 [SMILES: Cc1ccc(C2=C(COc3ccc(CCC(=O)O)c(C)c3C)COCC2)cc1F]

¹H NMR (300 MHz, CD₃OD) δ: 7.19 (t, J=8.1 Hz, 1H), 6.85-6.96 (m, 3H), 6.45 (d, J=8.4 Hz, 1H), 4.35 (s, 4H), 3.90 (t, J=5.4 Hz, 2H), 2.88 (t, J=7.5 Hz, 2H), 2.46-2.51 (m, 4H), 2.25 (s, 3H), 2.21 (s, 3H), 2.14 (s, 3H). ¹⁹F NMR (300 MHz, CD₃OD) δ: −77.04, −119.56. Mass spectrum (ESI, m/z): Calculated for C₂₄H₂₇FO₄, 397.2 [M-0.1CF₃COOH-H], Measured 397.2.

### Example 45

### 3-[4-[[4-(2,4-difluorophenyl)-3,6-dihydro-2H-pyran-5-yl]methoxy]-2,3-dimethyl-phenyl]propanoic acid

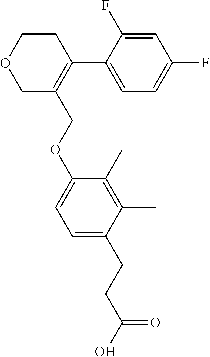 [SMILES: Cc1c(CCC(=O)O)ccc(OCC2=C(c3ccc(F)cc3F)CCOC2)c1C]

¹H NMR (300 MHz, CD₃OD) δ: 7.12-7.27 (m, 2H), 7.02-7.07 (m, 1H), 6.88 (d, J=8.4 Hz, 1H), 6.47 (d, J=8.4 Hz, 1H), 4.36 (s, 2H), 4.33 (s, 2H), 3.91 (t, J=5.4 Hz, 2H), 2.88 (t, J=7.5 Hz, 2H), 2.45-2.50 (m, 4H), 2.21 (s, 3H), 2.14 (s, 3H). Mass spectrum (ESI, m/z): Calculated for C_{23.4}H_{24.2}F_{2.6}O_{4.4}, 401.2 [M-0.2CF₃COOH-H], Measured 401.2.

### Example 46

### 3-[4-[[4-(4-fluoro-3-methyl-phenyl)-3,6-dihydro-2H-pyran-5-yl]methoxy]-2,3-dimethyl-phenyl]propanoic acid

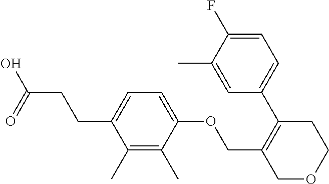 [SMILES: Cc1cc(C2=C(COc3ccc(CCC(=O)O)c(C)c3C)COCC2)ccc1F]

¹H NMR (300 MHz, CD₃OD) δ: 6.96-7.02 (m, 3H), 6.86 (d, J=8.4 Hz, 1H), 6.44 (d, J=8.4 Hz, 1H), 4.36 (s, 2H), 4.34 (s, 2H), 3.91 (t, J=7.5 Hz, 2H), 2.87 (t, J=7.6 Hz, 2H), 2.42-2.50 (m, 4H), 2.21 (s, 6H), 2.11 (s, 3H). ¹⁹F NMR (300 MHz, CD₃OD) δ: −121.47. Mass spectrum (ESI, m/z): Calculated for C₂₄H₂₇F₁O₄, 397.2 [M−H], Measured 397.1.

### Example 47

### 3-[4-[[4-(4-chloro-2-methoxy-phenyl)-3,6-dihydro-2H-pyran-5-yl]methoxy]-2,3-dimethyl-phenyl]propanoic acid

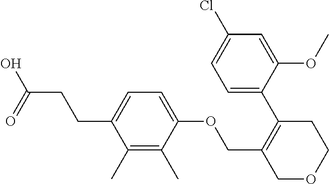 [SMILES: COc1cc(Cl)ccc1C1=C(COc2ccc(CCC(=O)O)c(C)c2C)COCC1]

¹H NMR (300 MHz, CD₃OD) δ: 7.02 (d, J=8.1 Hz, 1H), 6.93 (s, 1H), 6.92 (d, J=8.4 Hz, 1H), 6.83 (d, J=8.4 Hz, 1H), 6.14 (d, J=8.4 Hz, 1H), 4.25-4.35 (m, 2H), 4.25 (s, 2H), 3.89 (t, J=5.10 Hz, 2H), 3.78 (s, 3H), 2.85 (t, J=7.6 Hz, 2H), 2.40-2.49 (m, 4H), 2.19 (s, 3H), 2.13 (s, 3H). Mass spectrum (ESI, m/z): Calculated for C₂₄H₂₇ClO₅, 429.2 [M−H], Measured 428.9.

### Example 48

### 3-[4-[[4-(4-chloro-3-fluoro-phenyl)-3,6-dihydro-2H-pyran-5-yl]methoxy]-2,3-dimethyl-phenyl]propanoic acid

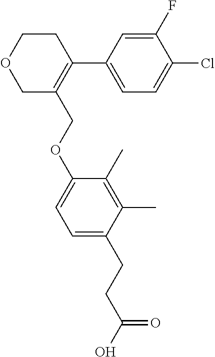 [SMILES: Cc1c(CCC(=O)O)ccc(OCC2=C(c3ccc(Cl)c(F)c3)CCOC2)c1C]

¹H NMR (300 MHz, D₃OD) δ: 7.43 (t, J=8.0 Hz, 1H), 7.03-7.15 (m, 2H), 6.87 (d, J=8.4 Hz, 1H), 6.45 (d, J=8.4 Hz, 1H), 4.36 (s, 2H), 4.33 (s, 2H), 3.91 (t, J=5.7 Hz, 2H), 2.87 (t, J=7.6 Hz, 2H), 2.45-2.50 (m, 4H), 2.21 (s, 3H), 2.14 (s, 3H). ¹⁹F NMR (300 MHz, CD₃OD) δ: −117.16. Mass spectrum (ESI, m/z): Calculated for C₂₃H₂₄ClFO₄, 417.1 [M−H], Measured 417.0.

### Example 49

### 3-[4-[[4-(4-chloro-3-methoxy-phenyl)-3,6-dihydro-2H-pyran-5-yl]methoxy]-2,3-dimethyl-phenyl]propanoic acid

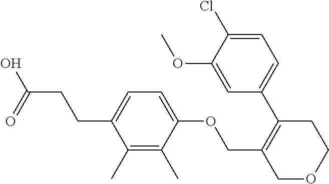 [SMILES: COc1cc(C2=C(COc3ccc(CCC(=O)O)c(C)c3C)COCC2)ccc1Cl]

¹H NMR (300 MHz, CD₃OD) δ: 7.02 (d, J=9.6 Hz, 1H), 6.94 (s, 1H), 6.91 (d, J=2.1 Hz, 1H), 6.83 (d, J=8.4 Hz, 1H), 6.14 (d, J=11.1 Hz, 1H), 4.38 (s, 2H), 4.29 (s, 2H), 3.91 (t, J=5.10 Hz, 2H), 3.80 (s, 3H), 2.86 (t, J=7.6 Hz, 2H), 2.40-2.49 (m, 4H), 2.19 (s, 3H), 2.14 (s, 3H). Mass spectrum (ESI, m/z): Calculated for C₂₄H₂₇ClO₅, 429.2 [M−H], Measured 429.1.

### Example 50

### 3-[2-(difluoromethyl)-4-[[4-(p-tolyl)-3,6-dihydro-2H-pyran-5-yl]methoxy]phenyl]propanoic acid

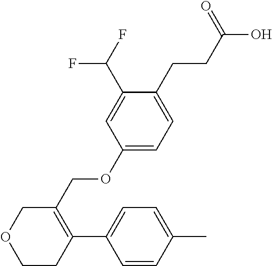 [SMILES: Cc1ccc(C2=C(COc3ccc(CCC(=O)O)c(C(F)F)c3)COCC2)cc1]

¹H NMR (CHLOROFORM-d) δ: 7.06-7.22 (m, 5H), 6.92 (d, J=2.5 Hz, 1H), 6.83 (dd, J=8.6, 2.5 Hz, 1H), 6.73 (t, J=55.3 Hz, 1H), 4.42 (s, 2H), 4.37 (s, 2H), 3.96 (t, J=5.6 Hz, 2H), 2.98 (t, J=7.8 Hz, 2H), 2.65 (t, J=7.6 Hz, 2H), 2.48 (m, 2H), 2.35 (s, 3H). Mass spectrum (ESI, m/z): Calculated for C₂₃H₂₄F₂O₄, 425.4 [M+Na], Measured 425.0.

### Example 51

### 3-[4-[[4-(D-tolyl)-3,6-dihydro-2H-pyran-5-yl]methoxy]-2,6-bis(trifluoromethyl)phenyl]propanoic acid

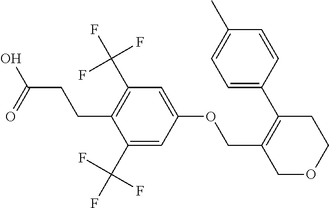 [SMILES: Cc1ccc(C2=C(COc3cc(C(F)(F)F)c(CCC(=O)O)c(C(F)(F)F)c3)COCC2)cc1]

¹H NMR (CHLOROFORM-d) δ: 7.24 (s, 2H), 7.16-7.20 (m, 2H), 7.07-7.11 (m, 2H), 4.49 (s, 2H), 4.33 (s, 2H), 3.94 (t, J=5.6 Hz, 2H), 3.15-3.27 (m, 2H), 2.58-2.68 (m, 2H), 2.49 (br s, 2H), 2.36 (s, 3H). Mass spectrum (ESI, m/z): Calculated for C₂₄H₂₂F₆O₄, 511.1 [M+Na], Measured 511.0.

### Example 52

### 3-[4-[[4-(4-chloro-2-fluoro-phenyl)-3,6-dihydro-2H-pyran-5-yl]methoxy]-2,3-dimethyl-phenyl]propanoic acid

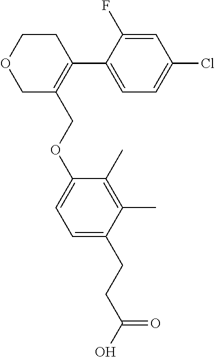 [SMILES: Cc1c(CCC(=O)O)ccc(OCC2=C(c3ccc(Cl)cc3F)CCOC2)c1C]

¹H NMR (300 MHz, CD₃OD) δ: 7.15-7.25 (m, 3H), 6.85 (d, J=8.4 Hz, 1H), 6.42 (d, J=8.4 Hz, 1H), 4.35 (s, 2H), 4.25 (s, 2H), 3.89 (t, J=5.10 Hz, 2H), 2.86 (t, J=7.6 Hz, 2H), 2.40-2.49 (m, 4H), 2.19 (s, 3H), 2.13 (s, 3H). ¹⁹F NMR (300 MHz, CD₃OD) δ: −114.41. Mass spectrum (ESI, m/z): Calculated for C₂₃H₂₄ClFO₄, 417.1 [M−H], Measured 417.1.

### Example 53

### 3-[4-[[4-(D-tolyl)-3,6-dihydro-2H-pyran-5-yl]methoxy]-2-(trifluoromethyl)phenyl]propanoic acid

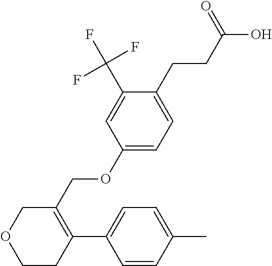 [SMILES: Cc1ccc(C2=C(COc3ccc(CCC(=O)O)c(C(F)(F)F)c3)COCC2)cc1]

¹H NMR (MeOH) δ: 7.29 (d, J=8.6 Hz, 1H), 7.07-7.20 (m, 4H), 7.00 (d, J=2.5 Hz, 1H), 6.92 (dd, J=8.1, 2.5 Hz, 1H), 4.45 (s, 2H), 4.30 (br d, J=2.0 Hz, 2H), 3.88 (t, J=5.6 Hz, 2H), 2.93-3.04 (m, 2H), 2.48-2.58 (m, 2H), 2.44 (br s, 2H), 2.32 (s, 3H). Mass spectrum (ESI, m/z): Calculated for C₂₃H₂₃F₃O₄, 443.1 [M+Na], Measured 443.0.

### Example 54

### 3-[2-chloro-4-[[4-(2,6-dimethylphenyl)-3,6-dihydro-2H-pyran-5-yl]methoxy]phenyl]propanoic acid

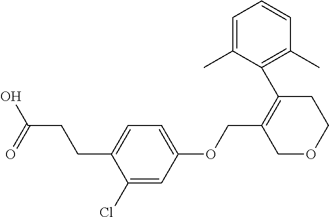 [SMILES: Cc1cccc(C)c1C1=C(COc2ccc(CCC(=O)O)c(Cl)c2)COCC1]

¹H NMR (CHLOROFORM-d) δ: 7.00-7.15 (m, 4H), 6.73 (d, J=2.5 Hz, 1H), 6.59 (dd, J=8.6, 2.5 Hz, 1H), 4.44 (br s, 2H), 4.11 (s, 2H), 3.99 (br t, J=5.1 Hz, 2H), 2.95 (t, J=7.6 Hz, 2H), 2.64 (t, J=7.6 Hz, 2H), 2.28 (br s, 2H), 2.20 (s, 6H). Mass spectrum (ESI, m/z): Calculated for C₂₃H₂₅ClO₄, 423.1 [M+Na], Measured 423.0.

### Example 55

### 3-[4-[[4-(5,5-dimethylcyclopenten-1-yl)-3,6-dihydro-2H-pyran-5-yl]methoxy]-2-(trifluoromethyl)phenyl]propanoic acid

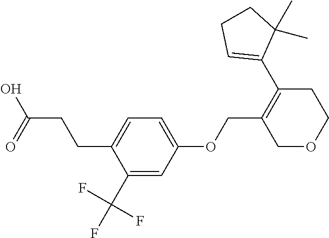 [SMILES: CC1(C)CCC=C1C1=C(COc2ccc(CCC(=O)O)c(C(F)(F)F)c2)COCC1]

¹H NMR (MeOH) δ: 7.35 (d, J=8.6 Hz, 1H), 7.11 (d, J=2.5 Hz, 1H), 7.06 (dd, J=8.3, 2.3 Hz, 1H), 5.37-5.43 (m, 1H), 4.49 (s, 2H), 4.27 (br d, J=2.0 Hz, 2H), 3.76-3.83 (m, 2H), 2.94-3.06 (m, 2H), 2.50-2.61 (m, 2H), 2.21-2.36 (m, 4H), 1.76 (t, J=7.1 Hz, 2H), 1.09 (s, 6H). Mass spectrum (ESI, m/z): Calculated for C₂₃H₂₇F₃O₄, 447.1 [M+Na], Measured 447.0.

### Example 56

### 3-[4-[[4-(2,6-dimethylphenyl)-3,6-dihydro-2H-pyran-5-yl]methoxy]-2-(trifluoromethyl)phenyl]propanoic acid

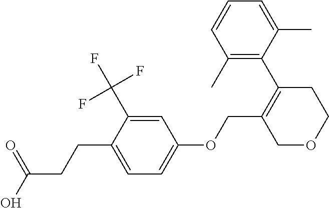 [SMILES: Cc1cccc(C)c1C1=C(COc2ccc(CCC(=O)O)c(C(F)(F)F)c2)COCC1]

¹H NMR (CHLOROFORM-d) δ: 7.17 (d, J=8.6 Hz, 1H), 6.98-7.14 (m, 4H), 6.82 (dd, J=8.6, 2.5 Hz, 1H), 4.47 (br s, 2H), 4.15 (s, 2H), 4.02 (br t, J=5.1 Hz, 2H), 3.03 (br t, J=7.6 Hz, 2H), 2.62 (br t, J=7.8 Hz, 2H), 2.30 (br s, 2H), 2.20 (s, 6H). Mass spectrum (ESI, m/z): Calculated for C₂₄H₂₅F₃O₄, 457.1 [M+Na], Measured 457.3.

### Example 57

### 3-[4-[[4-(2,2-difluoro-1,3-benzodioxol-4-yl)-3,6-dihydro-2H-pyran-5-yl]methoxy]-2-(trifluoromethyl)phenyl]propanoic acid

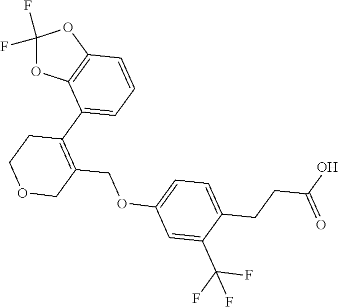 [SMILES: O=C(O)CCc1ccc(OCC2=C(c3cccc4c3OC(F)(F)O4)CCOC2)cc1C(F)(F)F]

¹H NMR (CHLOROFORM-d) δ: 7.21 (d, J=8.1 Hz, 1H), 6.99-7.12 (m, 3H), 6.93 (dd, J=7.8, 1.3 Hz, 1H), 6.86 (dd, J=8.6, 2.5 Hz, 1H), 4.42 (s, 2H), 4.39 (s, 2H), 3.95 (t, J=5.6 Hz, 2H), 3.05 (br t, J=7.8 Hz, 2H), 2.63 (t, J=7.8 Hz, 2H), 2.50 (br s, 2H). Mass spectrum (ESI, m/z): Calculated for C₂₃H₁₉F₅O₆, 509.1 [M+Na], Measured 509.0.

### Example 58

### 3-[4-[[4-[2-(trifluoromethoxy)phenyl]-3,6-dihydro-2H-pyran-5-yl]methoxy]-2-(trifluoromethyl)phenyl]propanoic acid

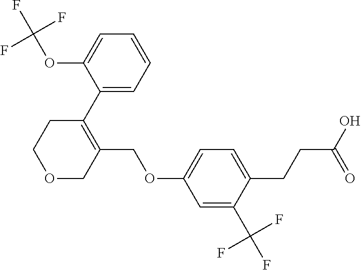 [SMILES: O=C(O)CCc1ccc(OCC2=C(c3ccccc3OC(F)(F)F)CCOC2)cc1C(F)(F)F]

¹H NMR (CHLOROFORM-d) δ: 7.16-7.40 (m, 5H), 6.99 (d, J=2.5 Hz, 1H), 6.85 (dd, J=8.6, 2.5 Hz, 1H), 4.32-4.45 (m, 2H), 4.28 (s, 2H), 3.82-4.05 (m, 2H), 3.03 (t, J=7.8 Hz, 2H), 2.57-2.69 (m, 2H), 2.41 (br s, 2H). Mass spectrum (ESI, m/z): Calculated for C₂₃H₂₀F₆O₅, 513.1 [M+Na], Measured 513.0.

### Example 59

### 3-[4-[[4-(2-methoxyphenyl)-3,6-dihydro-2H-pyran-5-yl]methoxy]-2-(trifluoromethyl)phenyl]propanoic acid

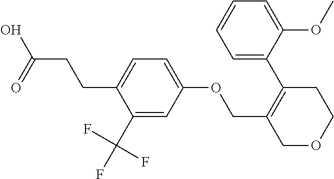 [SMILES: COc1ccccc1C1=C(COc2ccc(CCC(=O)O)c(C(F)(F)F)c2)COCC1]

¹H NMR (CHLOROFORM-d) δ: 7.29 (td, J=7.8, 1.5 Hz, 1H), 7.17 (d, J=8.6 Hz, 1H), 7.07 (dd, J=7.3, 1.8 Hz, 1H), 7.01 (d, J=2.5 Hz, 1H), 6.90-6.97 (m, 2H), 6.86 (dd, J=8.6, 2.5 Hz, 1H), 4.31-4.46 (m, 4H), 3.90-4.02 (m, 2H), 3.81 (s, 3H), 3.03 (br t, J=7.8 Hz, 2H), 2.63 (t, J=7.8 Hz, 2H). Mass spectrum (ESI, m/z): Calculated for C₂₃H₂₃F₃O₅, 459.1 [M+Na], Measured 459.0.

### Example 60

### 3-[4-[[4-(2-isobutoxyphenyl)-3,6-dihydro-2H-pyran-5-yl]methoxy]-2-(trifluoromethyl)phenyl]propanoic acid

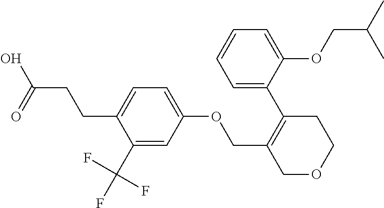 [SMILES: CC(C)COc1ccccc1C1=C(COc2ccc(CCC(=O)O)c(C(F)(F)F)c2)COCC1]

¹H NMR (CHLOROFORM-d) δ: 7.22-7.31 (m, 1H), 7.17 (d, J=8.6 Hz, 1H), 7.07 (dd, J=7.6, 1.5 Hz, 1H), 7.00 (d, J=2.5 Hz, 1H), 6.86-6.95 (m, 2H), 6.84 (dd, J=8.6, 2.5 Hz, 1H), 4.25-4.49 (m, 4H), 3.81-4.09 (m, 2H), 3.72 (br s, 2H), 3.03 (br t, J=7.6 Hz, 2H), 2.62 (br t, J=7.8 Hz, 2H), 2.27-2.57 (m, 2H), 2.05 (dquin, J=13.1, 6.6 Hz, 1H), 1.00 (d, J=7.1 Hz, 6H). Mass spectrum (ESI, m/z): Calculated for C₂H₂₉F₃O₅, 501.1 [M+Na], Measured 501.3.

### Example 61

### 3-[4-[(4-benzyl-3,6-dihydro-2H-pyran-5-yl)methoxy]-2-(trifluoromethyl)phenyl]propanoic acid

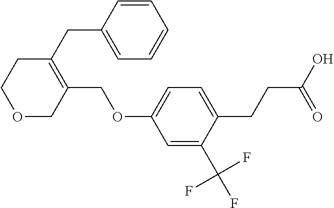 [SMILES: O=C(O)CCc1ccc(OCC2=C(Cc3ccccc3)CCOC2)cc1C(F)(F)F]

¹H NMR (CHLOROFORM-d) δ: 7.13-7.35 (m, 7H), 7.02 (dd, J=8.6, 2.5 Hz, 1H), 4.59 (s, 2H), 4.29 (s, 2H), 3.78 (t, J=5.6 Hz, 2H), 3.52 (s, 2H), 3.07 (br t, J=7.8 Hz, 2H), 2.64 (t, J=7.8 Hz, 2H), 2.08 (br s, 2H). Mass spectrum (ESI, m/z): Calculated for C₂₃H₂₃F₃O₄, 443.1 [M+Na], Measured 443.0.

### Example 62

### 3-[4-[[4-(4-chloro-3-methyl-phenyl)-3,6-dihydro-2H-pyran-5-yl]methoxy]-2-(trifluoromethyl)phenyl]propanoic acid

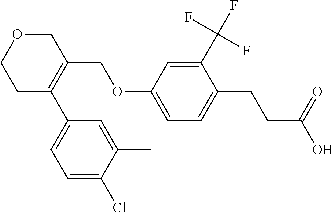 [SMILES: Cc1cc(C2=C(COc3ccc(CCC(=O)O)c(C(F)(F)F)c3)COCC2)ccc1Cl]

¹H NMR (CHLOROFORM-d) δ: 7.31 (d, J=8.1 Hz, 1H), 7.22 (d, J=8.6 Hz, 1H), 7.07 (d, J=2.0 Hz, 1H), 7.03 (d, J=2.5 Hz, 1H), 6.97 (dd, J=8.1, 2.0 Hz, 1H), 6.88 (dd, J=8.6, 2.5 Hz, 1H), 4.38 (s, 2H), 4.33 (s, 2H), 3.92 (t, J=5.6 Hz, 2H), 3.05 (t, J=7.8 Hz, 2H), 2.57-2.68 (m, 2H), 2.45 (br s, 2H), 2.34 (s, 3H). Mass spectrum (ESI, m/z): Calculated for C₂₂H₂₂ClF₃O₄, 477.1 [M+Na], Measured 477.0.

### Example 63

### 3-[4-[[4-(3,4-dichlorophenyl)-3,6-dihydro-2H-pyran-5-yl]methoxy]-2-trifluoromethyl)phenyl]propanoic acid

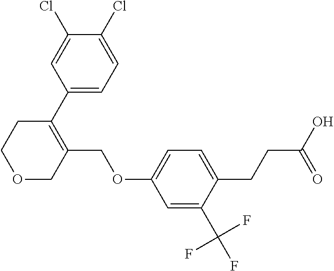 [SMILES: O=C(O)CCc1ccc(OCC2=C(c3ccc(Cl)c(Cl)c3)CCOC2)cc1C(F)(F)F]

¹H NMR (CHLOROFORM-d) δ: 7.42 (d, J=8.1 Hz, 1H), 7.32 (d, J=2.0 Hz, 1H), 7.23 (d, J=8.6 Hz, 1H), 7.01-7.09 (m, 2H), 6.89 (dd, J=8.6, 2.5 Hz, 1H), 4.36 (s, 4H), 3.95 (t, J=5.6 Hz, 2H), 3.06 (t, J=7.8 Hz, 2H), 2.59-2.69 (m, 2H), 2.45 (br s, 2H). Mass spectrum (ESI, m/z): Calculated for C₂₂H₁₉Cl₂F₃O₄, 497.1 [M+Na], Measured 497.0.

### Example 64

### 3-[2-(trifluoromethyl)-4-[[4-(2,4,5-trimethylphenyl)-3,6-dihydro-2H-pyran-5-yl]methoxy]phenyl]propanoic acid

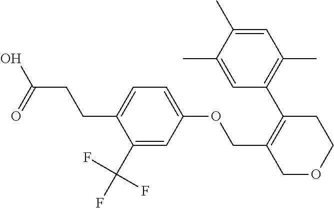 [SMILES: Cc1cc(C)c(C2=C(COc3ccc(CCC(=O)O)c(C(F)(F)F)c3)COCC2)cc1C]

¹H NMR (CHLOROFORM-d) δ: 7.18 (d, J=8.1 Hz, 1H), 7.00 (d, J=2.5 Hz, 1H), 6.97 (s, 1H), 6.84 (dd, J=8.3, 2.8 Hz, 1H), 6.81 (s, 1H), 4.31-4.47 (m, 2H), 4.27 (s, 2H), 3.95 (t, J=5.6 Hz, 2H), 3.03 (t, J=7.8 Hz, 2H), 2.56-2.68 (m, 2H), 2.34 (br s, 2H), 2.22 (s, 3H), 2.20 (s, 3H), 2.14 (s, 3H). Mass spectrum (ESI, m/z): Calculated for C₂₅H₂₇F₃O₄, 471.1 [M+Na], Measured 471.0.

### Example 65

### 3-[4-[[4-(4-chloro-2-methyl-phenyl)-3,6-dihydro-2H-pyran-5-yl]methoxy]-2-(trifluoromethyl)phenyl]propanoic acid

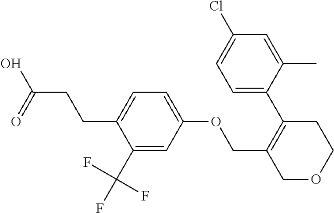 [SMILES: Cc1cc(Cl)ccc1C1=C(COc2ccc(CCC(=O)O)c(C(F)(F)F)c2)COCC1]

¹H NMR (CHLOROFORM-d) δ: 7.12-7.23 (m, 3H), 6.97-7.03 (m, 2H), 6.96 (s, 1H), 6.83 (dd, J=8.6, 2.5 Hz, 1H), 4.31-4.49 (m, 2H), 4.21 (s, 2H), 3.96 (t, J=5.6 Hz, 2H), 3.04 (t, J=7.8 Hz, 2H), 2.55-2.69 (m, 2H), 2.32 (br d, J=1.5 Hz, 2H), 2.21 (s, 3H). Mass spectrum (ESI, m/z): Calculated for C₂₃H₂₂ClF₃O₄, 477.1 [M+Na], Measured 477.0.

### Example 66

### 3-[4-[[4-(3,4-dimethylphenyl)-3,6-dihydro-2H-pyran-5-yl]methoxy]-2-(trifluoromethyl)phenyl]propanoic acid

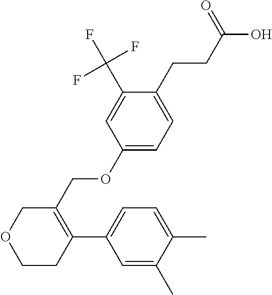 [SMILES: Cc1ccc(C2=C(COc3ccc(CCC(=O)O)c(C(F)(F)F)c3)COCC2)cc1C]

¹H NMR (CHLOROFORM-d) δ: 7.20 (d, J=8.6 Hz, 1H), 7.11 (d, J=8.1 Hz, 1H), 7.03 (d, J=2.5 Hz, 1H), 6.98 (s, 1H), 6.94 (d, J=7.6 Hz, 1H), 6.88 (dd, J=8.6, 2.5 Hz, 1H), 4.43 (s, 2H), 4.35 (s, 2H), 3.93 (t, J=5.6 Hz, 2H), 3.04 (br t, J=8.1 Hz, 2H), 2.58-2.68 (m, 2H), 2.47 (br s, 2H), 2.26 (s, 3H), 2.23 (s, 3H). Mass spectrum (ESI, m/z): Calculated for C₂₄H₂₅F₃O₄, 457.1 [M+Na], Measured 457.0.

### Example 67

### 3-[4-[[4-(2-fluoro-4-methyl-phenyl)-3,6-dihydro-2H-pyran-5-yl]methoxy]-2-(trifluoromethyl)phenyl]propanoic acid

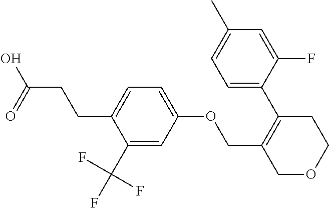 [SMILES: Cc1ccc(C2=C(COc3ccc(CCC(=O)O)c(C(F)(F)F)c3)COCC2)c(F)c1]

¹H NMR (CHLOROFORM-d) δ: 7.20 (d, J=8.6 Hz, 1H), 6.99-7.08 (m, 2H), 6.89-6.97 (m, 2H), 6.86 (dd, J=8.6, 2.5 Hz, 1H), 4.35-4.43 (m, 4H), 3.96 (br t, J=5.1 Hz, 2H), 3.04 (br t, J=7.6 Hz, 2H), 2.64 (br t, J=7.6 Hz, 2H), 2.45 (br s, 2H), 2.35 (s, 3H). Mass spectrum (ESI, m/z): Calculated for C₂₃H₂₂F₄O₄, 461.1 [M+Na], Measured 461.0.

### Example 68

### 3-[4-[[4-(3-fluoro-4-methyl-phenyl)-3,6-dihydro-2H-pyran-5-yl]methoxy]-2-(trifluoromethyl)phenyl]propanoic acid

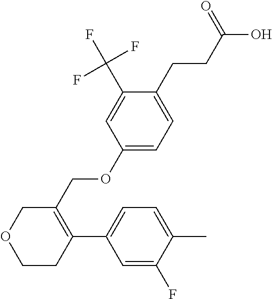 [SMILES: Cc1ccc(C2=C(COc3ccc(CCC(=O)O)c(C(F)(F)F)c3)COCC2)cc1F]

¹H NMR (CHLOROFORM-d) δ: 7.21 (d, J=8.6 Hz, 1H), 7.15 (t, J=7.8 Hz, 1H), 7.03 (d, J=2.5 Hz, 1H), 6.83-6.91 (m, 3H), 4.41 (s, 2H), 4.35 (s, 2H), 3.94 (t, J=5.6 Hz, 2H), 3.05 (t, J=7.8 Hz, 2H), 2.60-2.68 (m, 2H), 2.46 (br s, 2H), 2.27 (d, J=1.5 Hz, 3H). Mass spectrum (ESI, m/z): Calculated for C₂₃H₂₂F₄O₄, 461.1 [M+Na], Measured 461.0.

### Example 69

### 3-[4-[[4-[[4-(trifluoromethoxy)phenyl]methyl]-3,6-dihydro-2H-pyran-5-yl]methoxy]-2-(trifluoromethyl)phenyl]propanoic acid

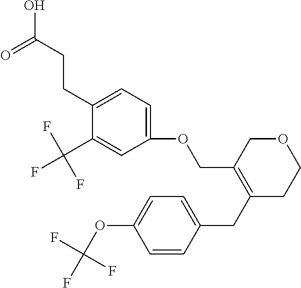 [SMILES: O=C(O)CCc1ccc(OCC2=C(Cc3ccc(OC(F)(F)F)cc3)CCOC2)cc1C(F)(F)F]

¹H NMR (CHLOROFORM-d) δ: 7.24-7.30 (m, 1H), 7.11-7.22 (m, 5H), 7.01 (dd, J=8.3, 2.3 Hz, 1H), 4.56 (s, 2H), 4.30 (br s, 2H), 3.80 (t, J=5.6 Hz, 2H), 3.51 (s, 2H), 3.07 (br t, J=7.6 Hz, 2H), 2.65 (br t, J=7.8 Hz, 2H), 2.07 (br s, 2H). Mass spectrum (ESI, m/z): Calculated for C₂₄H₂₂FO₅, 527.1 [M+Na], Measured 527.0.

### Example 70

### 3-[4-[[4-(1-phenylvinyl)-3,6-dihydro-2H-pyran-5-yl]methoxy]-2-(trifluoromethyl)phenyl]propanoic acid

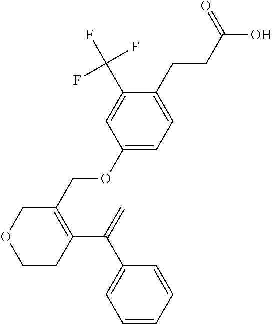 [SMILES: C=C(C1=C(COc2ccc(CCC(=O)O)c(C(F)(F)F)c2)COCC1)c1ccccc1]

¹H NMR (CHLOROFORM-d) δ: 7.28-7.42 (m, 5H), 7.20 (d, J=8.6 Hz, 1H), 7.09 (d, J=2.5 Hz, 1H), 6.92 (dd, J=8.3, 2.3 Hz, 1H), 5.59 (s, 1H), 5.14 (s, 1H), 4.53 (s, 2H), 4.40 (s, 2H), 3.87 (t, J=5.3 Hz, 2H), 3.04 (br t, J=7.8 Hz, 2H), 2.63 (t, J=7.8 Hz, 2H), 2.23 (br s, 2H). Mass spectrum (ESI, m/z): Calculated for C₂₄H₂₃F₃O₄, 455.1 [M+Na], Measured 455.0.

### Example 71

### 3-[4-[(4-benzyl-3,6-dihydro-2H-pyran-5-yl)methoxy]phenyl]propanoic acid

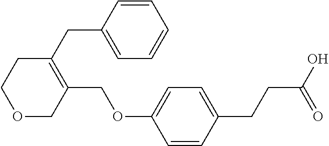 [SMILES: O=C(O)CCc1ccc(OCC2=C(Cc3ccccc3)CCOC2)cc1]

¹H NMR (CHLOROFORM-d) δ: 7.27-7.33 (m, 2H), 7.15-7.24 (m, 3H), 7.13 (d, J=8.1 Hz, 2H), 6.86 (d, J=8.6 Hz, 2H), 4.56 (s, 2H), 4.30 (s, 2H), 3.77 (t, J=5.6 Hz, 2H), 3.51 (s, 2H), 2.86-2.95 (m, 2H), 2.61-2.69 (m, 2H), 2.03-2.10 (m, 2H). Mass spectrum (ESI, m/z): Calculated for C₂₂H₂₄O₄, 375.1 [M+Na], Measured 375.0.

### Example 72

### 3-[4-[[4-(D-tolylmethyl)-3,6-dihydro-2H-pyran-5-yl]methoxy]phenyl]propanoic acid

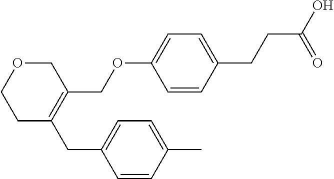 [SMILES: Cc1ccc(CC2=C(COc3ccc(CCC(=O)O)cc3)COCC2)cc1]

¹H NMR (CHLOROFORM-d) δ: 7.02-7.17 (m, 6H), 6.86 (d, J=8.6 Hz, 2H), 4.55 (s, 2H), 4.28 (s, 2H), 3.76 (t, J=5.6 Hz, 2H), 3.46 (s, 2H), 2.83-2.96 (m, 2H), 2.60-2.70 (m, 2H), 2.32 (s, 3H), 2.05 (br s, 2H). Mass spectrum (ESI, m/z): Calculated for C₂₃H₂₆O₄, 389.1 [M+Na], Measured 389.0.

### Example 73

### 3-[4-[[4-(p-tolylmethyl)-3,6-dihydro-2H-pyran-5-yl]methoxy]-2-(trifluoromethyl)phenyl]propanoic acid

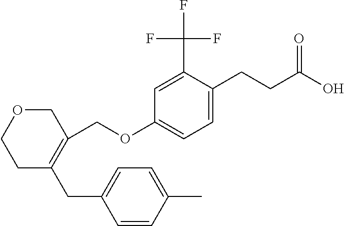 [SMILES: Cc1ccc(CC2=C(COc3ccc(CCC(=O)O)c(C(F)(F)F)c3)COCC2)cc1]

¹H NMR (CHLOROFORM-d) δ: 7.23-7.29 (m, 1H), 7.17 (d, J=2.5 Hz, 1H), 7.03-7.13 (m, 4H), 7.02 (dd, J=8.6, 2.5 Hz, 1H), 4.58 (s, 2H), 4.28 (s, 2H), 3.77 (t, J=5.6 Hz, 2H), 3.47 (s, 2H), 3.07 (t, J=7.8 Hz, 2H), 2.60-2.69 (m, 2H), 2.32 (s, 3H), 2.07 (br s, 2H). Mass spectrum (ESI, m/z): Calculated for C₂₄H₂₅F₃O₄, 457.1 [M+Na], Measured 457.0.

### Example 74

### 3-[4-[[4-[(4-chlorophenyl)methyl]-3,6-dihydro-2H-pyran-5-yl]methoxy]phenyl]propanoic acid

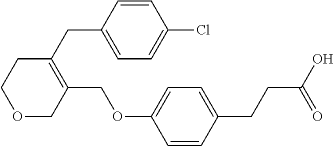 [SMILES: O=C(O)CCc1ccc(OCC2=C(Cc3ccc(Cl)cc3)CCOC2)cc1]

¹H NMR (CHLOROFORM-d) δ: 7.26 (m, 2H), 7.12 (t, J=8.6 Hz, 4H), 6.85 (d, J=8.6 Hz, 2H), 4.52 (s, 2H), 4.28 (s, 2H), 3.76 (t, J=5.6 Hz, 2H), 3.47 (s, 2H), 2.83-2.97 (m, 2H), 2.59-2.72 (m, 2H), 2.03 (br s, 3H). Mass spectrum (ESI, m/z): Calculated for C₂₂H₂₃ClO₄, 409.1 [M+Na], Measured 409.0.

### Example 75

### 3-[4-[[4-[(4-chlorophenyl)methyl]-3,6-dihydro-2H-pyran-5-yl]methoxy]-2-(trifluoromethyl)phenyl]propanoic acid

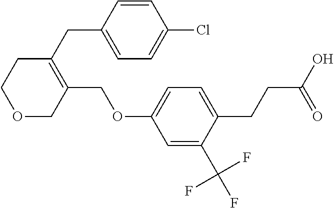 [SMILES: O=C(O)CCc1ccc(OCC2=C(Cc3ccc(Cl)cc3)CCOC2)cc1C(F)(F)F]

¹H NMR (CHLOROFORM-d) δ: 7.21-7.31 (m, 3H), 7.17 (d, J=2.5 Hz, 1H), 7.10 (d, J=8.1 Hz, 2H), 7.01 (dd, J=8.6, 2.5 Hz, 1H), 4.56 (s, 2H), 4.31 (br s, 2H), 3.81 (t, J=5.6 Hz, 2H), 3.48 (s, 2H), 3.08 (br t, J=7.8 Hz, 2H), 2.66 (t, J=7.8 Hz, 2H), 2.06 (br s, 2H). Mass spectrum (ESI, m/z): Calculated for C₂₃H₂₂ClF₃O₄, 477.1 [M+Na], Measured 477.0.

### Example 76

### 3-[4-[[4-[(4-methoxyphenyl)methyl]-3,6-dihydro-2H-pyran-5-yl]methoxy]phenyl]propanoic acid

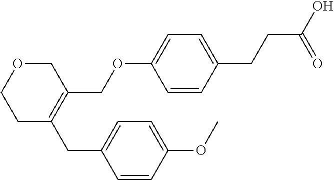 [SMILES: COc1ccc(CC2=C(COc3ccc(CCC(=O)O)cc3)COCC2)cc1]

¹H NMR (CHLOROFORM-d) δ: 7.10 (dd, J=16.7, 8.6 Hz, 4H), 6.84 (dd, J=12.9, 8.3 Hz, 4H), 4.55 (s, 2H), 4.28 (br s, 2H), 3.69-3.89 (m, 5H), 3.44 (s, 2H), 2.80-3.02 (m, 2H), 2.53-2.73 (m, 2H), 2.05 (br s, 2H). Mass spectrum (ESI, m/z): Calculated for C₂₃H₂O₅, 405.1 [M+Na], Measured 405.0.

### Example 77

### 3-[4-[[4-[(4-methoxyphenyl)methyl]-3,6-dihydro-2H-pyran-5-yl]methoxy]-2-(trifluoromethyl)phenyl]propanoic acid

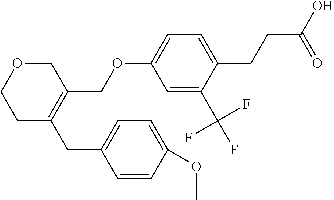 [SMILES: COc1ccc(CC2=C(COc3ccc(CCC(=O)O)c(C(F)(F)F)c3)COCC2)cc1]

¹H NMR (CHLOROFORM-d) δ: 7.24-7.31 (m, 1H), 7.18 (d, J=2.0 Hz, 1H), 7.08 (d, J=8.6 Hz, 2H), 7.02 (br d, J=8.6 Hz, 1H), 6.84 (d, J=8.6 Hz, 2H), 4.59 (s, 2H), 4.31 (br s, 2H), 3.74-3.83 (m, 5H), 3.45 (s, 2H), 3.07 (br t, J=7.6 Hz, 2H), 2.66 (t, J=7.8 Hz, 2H), 2.08 (br s, 2H). Mass spectrum (ESI, m/z): Calculated for C₂₄H₂₅F₃O₅, 473.1 [M+Na], Measured 473.0.

### Example 78

### 3-[4-[[4-(1-phenylvinyl)-3,6-dihydro-2H-pyran-5-yl]methoxy]phenyl]propanoic acid

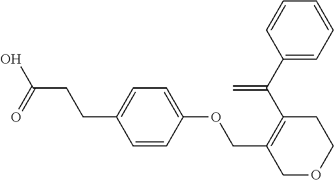 [SMILES: C=C(C1=C(COc2ccc(CCC(=O)O)cc2)COCC1)c1ccccc1]

¹H NMR (CHLOROFORM-d) δ: 7.35 (br d, J=11.1 Hz, 5H), 7.06 (br d, J=8.1 Hz, 2H), 6.77 (br d, J=8.1 Hz, 2H), 5.57 (s, 1H), 5.14 (s, 1H), 4.51 (s, 2H), 4.42 (br s, 2H), 3.86 (br s, 2H), 2.84-2.92 (m, 2H), 2.63 (br t, J=7.6 Hz, 2H), 2.21 (br s, 2H). Mass spectrum (ESI, m/z): Calculated for C₂₄H₂₅F₃O₅, 387.1 [M+Na], Measured 387.0.

### Example 79

### 3-[4-[[4-(1-phenylethyl)-3,6-dihydro-2H-pyran-5-yl]methoxy]-2-(trifluoromethyl)phenyl]propanoic acid

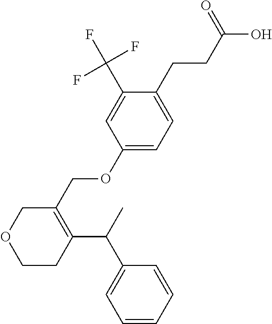 [SMILES: CC(C1=C(COc2ccc(CCC(=O)O)c(C(F)(F)F)c2)COCC1)c1ccccc1]

¹H NMR (CHLOROFORM-d) δ: 7.14-7.35 (m, 7H), 7.03 (dd, J=8.3, 2.8 Hz, 1H), 4.60 (s, 2H), 4.30 (s, 2H), 4.17 (q, J=7.1 Hz, 1H), 3.84 (dt, J=11.0, 5.4 Hz, 1H), 3.69 (ddd, J=11.2, 6.9, 4.5 Hz, 1H), 3.08 (br t, J=7.8 Hz, 2H), 2.61-2.70 (m, 2H), 2.17 (br d, J=17.2 Hz, 1H), 1.77 (br d, J=17.2 Hz, 1H), 1.44 (d, J=7.1 Hz, 3H).). Mass spectrum (ESI, m/z): Calculated for C₂₄H₂₅F₃O₄, 457.1 [M+Na], Measured 457.0.

### Example 80

### 3-[4-[[4-[1-(4-fluorophenyl)vinyl]-3,6-dihydro-2H-pyran-5-yl]methoxy]phenyl]propanoic acid

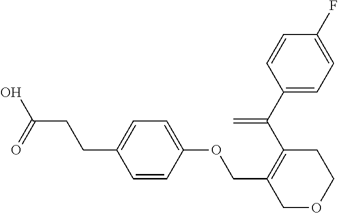 [SMILES: C=C(C1=C(COc2ccc(CCC(=O)O)cc2)COCC1)c1ccc(F)cc1]

¹H NMR (CHLOROFORM-d) δ: 7.30-7.39 (m, 2H), 7.07 (d, J=8.6 Hz, 2H), 6.95-7.05 (m, 2H), 6.76 (d, J=8.6 Hz, 2H), 5.50 (d, J=1.0 Hz, 1H), 5.11 (s, 1H), 4.49 (s, 2H), 4.40 (s, 2H), 3.84 (t, J=5.6 Hz, 2H), 2.80-2.93 (m, 2H), 2.52-2.68 (m, 2H), 2.18 (br s, 2H). Mass spectrum (ESI, m/z): Calculated for C₂₃H₂₃FO₄, 405.1 [M+Na], Measured 405.0.

### Example 81

### 3-[4-[[4-[1-(3-fluorophenyl)vinyl]-3,6-dihydro-2H-pyran-5-yl]methoxy]-2-(trifluoromethyl)phenyl]propanoic acid

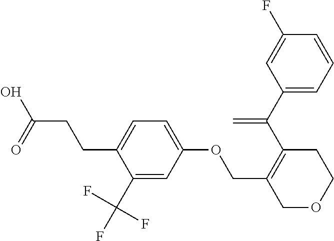 [SMILES: C=C(C1=C(COc2ccc(CCC(=O)O)c(C(F)(F)F)c2)COCC1)c1cccc(F)c1]

¹H NMR (CHLOROFORM-d) δ: 7.27-7.34 (m, 1H), 7.21 (d, J=8.6 Hz, 1H), 7.15 (d, J=7.6 Hz, 1H), 7.09 (d, J=3.0 Hz, 1H), 7.05 (dt, J=10.2, 2.0 Hz, 1H), 6.99 (td, J=8.3, 2.0 Hz, 1H), 6.92 (dd, J=8.6, 2.5 Hz, 1H), 5.61 (s, 1H), 5.19 (s, 1H), 4.50 (s, 2H), 4.41 (s, 2H), 3.89 (t, J=5.6 Hz, 2H), 3.05 (br t, J=7.8 Hz, 2H), 2.64 (t, J=7.8 Hz, 2H), 2.23 (br s, 2H). Mass spectrum (ESI, m/z): Calculated for C₂₄H₂₂F₄O₄, 473.1 [M+Na], Measured 473.1.

### Example 82

### 3-[2-(trifluoromethyl)-4-[[4-[1-[4-(trifluoromethyl)phenyl]vinyl]-3,6-dihydro-2H-pyran-5-yl]methoxy]phenyl]propanoic acid

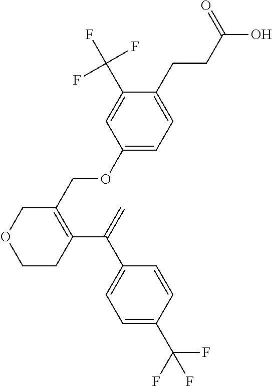 [SMILES: C=C(C1=C(COc2ccc(CCC(=O)O)c(C(F)(F)F)c2)COCC1)c1ccc(C(F)(F)F)cc1]

¹H NMR (CHLOROFORM-d) δ: 7.58 (d, J=8.1 Hz, 2H), 7.47 (d, J=8.1 Hz, 2H), 7.21 (d, J=8.6 Hz, 1H), 7.07 (d, J=2.5 Hz, 1H), 6.91 (dd, J=8.3, 2.8 Hz, 1H), 5.67 (d, J=1.0 Hz, 1H), 5.26 (d, J=1.0 Hz, 1H), 4.49 (s, 2H), 4.36-4.43 (m, 2H), 3.87 (t, J=5.6 Hz, 2H), 3.04 (t, J=7.8 Hz, 2H), 2.57-2.66 (m, 2H), 2.21 (br s, 2H). Mass spectrum (ESI, m/z): Calculated for C₂₅H₂₂F₆O₄, 523.1 [M+Na], Measured 523.1.

### Example 83

### 3-[4-[[4-[1-(2-chlorophenyl)vinyl]-3,6-dihydro-2H-pyran-5-yl]methoxy]-2-(trifluoromethyl)phenyl]propanoic acid

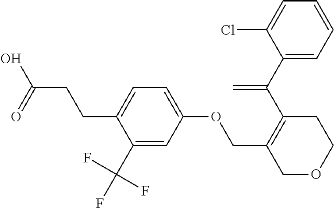 [SMILES: C=C(C1=C(COc2ccc(CCC(=O)O)c(C(F)(F)F)c2)COCC1)c1ccccc1Cl]

¹H NMR (CHLOROFORM-d) δ: 7.30-7.38 (m, 1H), 7.17-7.25 (m, 4H), 7.10 (d, J=2.5 Hz, 1H), 6.96 (dd, J=8.6, 2.5 Hz, 1H), 5.42 (d, J=1.5 Hz, 1H), 5.36 (d, J=1.5 Hz, 1H), 4.56 (s, 2H), 4.35 (s, 2H), 3.81 (t, J=5.6 Hz, 2H), 3.07 (br t, J=7.8 Hz, 2H), 2.59-2.69 (m, 2H), 2.14 (br s, 2H). Mass spectrum (ESI, m/z): Calculated for C₂₄H₂₂ClF₃O₄, 489.1 [M+Na], Measured 489.0.

### Example 84

### 3-[4-[(4-butyl-3,6-dihydro-2H-pyran-5-yl)methoxy]-2-(trifluoromethyl)phenyl]propanoic acid

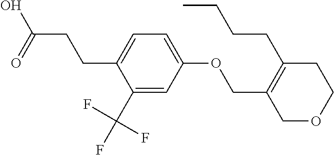 [SMILES: CCCCC1=C(COc2ccc(CCC(=O)O)c(C(F)(F)F)c2)COCC1]

¹H NMR (CHLOROFORM-d) δ: 7.21-7.30 (m, 1H), 7.17 (d, J=2.5 Hz, 1H), 7.01 (dd, J=8.1, 2.5 Hz, 1H), 4.47 (s, 2H), 4.19 (br s, 2H), 3.71-3.87 (m, 2H), 2.99-3.16 (m, 2H), 2.65 (t, J=7.8 Hz, 2H), 2.15 (br d, J=5.6 Hz, 4H), 1.17-1.47 (m, 4H), 0.90 (t, J=7.3 Hz, 3H). Mass spectrum (ESI, m/z): Calculated for C₂₀H₂₅F₃O₄, 409.1 [M+Na], Measured 409.0.

### Example 85

### 3-[2-chloro-4-[[4-[(4-chlorophenyl)methyl]-3,6-dihydro-2H-pyran-5-yl]methoxy]phenyl]propanoic acid

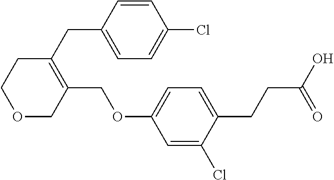 [SMILES: O=C(O)CCc1ccc(OCC2=C(Cc3ccc(Cl)cc3)CCOC2)cc1Cl]

¹H NMR (400 MHz, CD₃OD) δ: 7.19-7.30 (m, 5H), 7.01 (s, 1H), 6.86 (d, J=8.4 Hz, 1H), 4.63 (s, 2H), 4.25 (s, 2H), 3.74 (t, J=5.6 Hz, 2H), 3.53 (s, 2H), 2.99 (t, J=7.2 Hz, 2H), 2.98 (t, J=7.2 Hz, 2H), 2.04 (br s, 2H). Mass spectrum (ESI, m/z): Calculated for C₂₂H₂₂C₁₂O₄, 419.1 [M−H], Measured 419.0.

### Example 86

### 3-[4-[(4-benzyl-3,6-dihydro-2H-pyran-5-yl)methoxy]-2-chloro-phenyl]propanoic acid

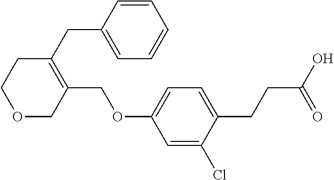 [SMILES: O=C(O)CCc1ccc(OCC2=C(Cc3ccccc3)CCOC2)cc1Cl]

¹H NMR (300 MHz, CD₃OD) δ: 7.15-7.29 (m, 6H), 6.84 (d, J=8.7 Hz, 1H), 4.90 (s, 2H), 4.23 (s, 2H), 3.72 (t, J=5.7 Hz, 2H), 3.52 (s, 2H), 2.96 (t, J=7.5 Hz, 2H), 2.57 (t, J=7.5 Hz, 2H), 2.03 (br s, 2H). Mass spectrum (ESI, m/z): Calculated for C₂₂H₂₃ClO₄, 385.1 [M−H], Measured 385.1.

### Example 87

### 3-[4-[(4-benzyl-3,6-dihydro-2H-pyran-5-yl)methoxy]-3,5-difluoro-phenyl]propanoic acid

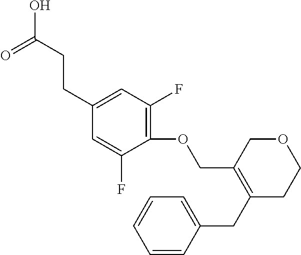 [SMILES: O=C(O)CCc1cc(F)c(OCC2=C(Cc3ccccc3)CCOC2)c(F)c1]

¹H NMR (300 MHz, CD₃OD) δ: 7.22-7.40 (m, 3H), 7.08-7.19 (m, 2H), 6.86-6.94 (m, 2H), 4.71 (s, 2H), 4.33 (s, 2H), 3.71 (t, J=5.7 Hz, 2H), 3.54 (s, 2H), 2.88 (t, J=7.8 Hz, 2H), 2.61 (t, J=7.8 Hz, 2H), 1.97-2.04 (m, 2H). ¹⁹F NMR (300 MHz, CD₃OD) δ: −76.99, −129.93. Mass spectrum (ESI, m/z): Calculated for C₂₂H₂₃F₂O₄, 387.1 [M−H], Measured 387.1.

### Example 88

### 3-[4-[(4-ethyl-3,6-dihydro-2H-pyran-5-yl)methoxy]-2-(trifluoromethyl)phenyl]propanoic acid

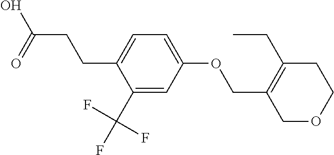 [SMILES: CCC1=C(COc2ccc(CCC(=O)O)c(C(F)(F)F)c2)COCC1]

¹H NMR (CHLOROFORM-d) δ: 7.26 (m, 1H), 7.17 (d, J=2.5 Hz, 1H), 6.98-7.05 (m, 1H), 4.48 (s, 2H), 4.19 (s, 2H), 3.82 (t, J=5.6 Hz, 2H), 3.08 (t, J=7.8 Hz, 2H), 2.60-2.71 (m, 2H), 2.11-2.23 (m, 4H), 1.03 (t, J=7.6 Hz, 3H). Mass spectrum (ESI, m/z): Calculated for C₁₈H₂₁F₃O₄, 381.1 [M+Na], Measured 381.1.

### Example 89

### 3-[4-[(4-isobutyl-3,6-dihydro-2H-pyran-5-yl)methoxy]-2-(trifluoromethyl)phenyl]propanoic acid

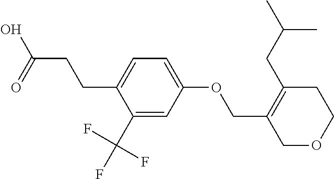 [SMILES: CC(C)CC1=C(COc2ccc(CCC(=O)O)c(C(F)(F)F)c2)COCC1]

¹H NMR (CHLOROFORM-d) δ: 7.26 (m, 1H), 7.16 (d, J=2.5 Hz, 1H), 7.00 (dd, J=8.6, 2.5 Hz, 1H), 4.47 (s, 2H), 4.22 (br s, 2H), 3.81 (t, J=5.6 Hz, 2H), 3.07 (br t, J=7.8 Hz, 2H), 2.65 (t, J=7.8 Hz, 2H), 2.14 (br s, 2H), 2.04 (d, J=7.1 Hz, 2H), 1.80 (dquin, J=13.6, 6.7 Hz, 1H), 0.90 (d, J=6.6 Hz, 6H). Mass spectrum (ESI, m/z): Calculated for C₂₀H₂₅F₃O₄, 409.1 [M+Na], Measured 409.1.

### Example 90

### 3-[4-[[4-(cyclohexylmethyl)-3,6-dihydro-2H-pyran-5-yl]methoxy]-2-(trifluoromethyl)phenyl]propanoic acid

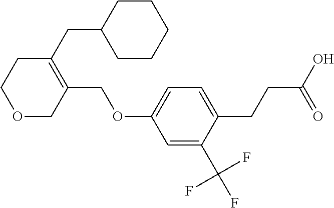 [SMILES: O=C(O)CCc1ccc(OCC2=C(CC3CCCCC3)CCOC2)cc1C(F)(F)F]

¹H NMR (CHLOROFORM-d) δ: 7.26-7.30 (m, 1H), 7.16 (d, J=2.5 Hz, 1H), 7.00 (dd, J=8.6, 2.5 Hz, 1H), 4.47 (s, 2H), 4.23 (br s, 2H), 3.83 (t, J=5.6 Hz, 2H), 3.08 (br t, J=7.6 Hz, 2H), 2.66 (t, J=7.8 Hz, 2H), 2.15 (br s, 2H), 2.04 (br d, J=7.1 Hz, 2H), 1.68 (br d, J=9.6 Hz, 5H), 1.43 (ddd, J=10.7, 7.5, 3.5 Hz, 1H), 1.04-1.29 (m, 3H), 0.77-0.95 (m, 2H).). Mass spectrum (ESI, m/z): Calculated for C₂₃H₂₉F₃O₄, 449.1 [M+Na], Measured 449.3.

### Example 91

### 3-[4-[(4-hexyl-3,6-dihydro-2H-pyran-5-yl)methoxy]-2-(trifluoromethyl)phenyl]propanoic acid

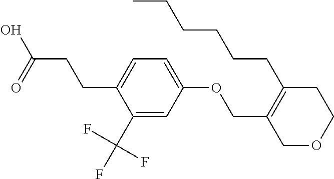 [SMILES: CCCCCCC1=C(COc2ccc(CCC(=O)O)c(C(F)(F)F)c2)COCC1]

¹H NMR (CHLOROFORM-d) δ: 7.24-7.29 (m, 1H), 7.16 (d, J=2.5 Hz, 1H), 7.01 (dd, J=8.6, 2.5 Hz, 1H), 4.47 (s, 2H), 4.20 (s, 2H), 3.81 (t, J=5.6 Hz, 2H), 3.07 (t, J=7.8 Hz, 2H), 2.57-2.71 (m, 2H), 2.15 (br d, J=7.6 Hz, 4H), 1.40 (br d, J=8.1 Hz, 2H), 1.27 (br s, 6H), 0.80-0.91 (m, 3H). Mass spectrum (ESI, m/z): Calculated for C₂₂H₂₉F₃O₄, 437.1 [M+Na], Measured 437.3.

### Example 92

### 3-(2-chloro-4-[[4-(1-phenylethenyl)-5,6-dihydro-2H-pyran-3-yl]methoxy]phenyl)propanoic acid

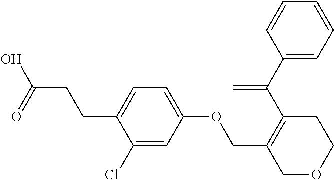 [SMILES: C=C(C1=C(COc2ccc(CCC(=O)O)c(Cl)c2)COCC1)c1ccccc1]

### Step 1. 4,4,5,5-tetramethyl-2-(1-phenylethenyl)-1,3,2-dioxaborolane

Into a 50-mL round-bottom flask purged and maintained with an inert atmosphere of nitrogen was added (1-bromoethenyl)benzene (800 mg, 4.370 mmol, 1.00 equiv), 4,4,5,5-tetramethyl-2-(tetramethyl-1,3,2-dioxaborolan-2-yl)-1,3,2-dioxaborolane (1.23 g, 4.840 mmol, 1.11 equiv), potassium phenolate (870 mg, 6.580 mmol, 1.51 equiv), Pd(PPh₃)₂Cl₂ (92 mg, 0.080 mmol, 0.02 equiv), PPh₃ (69 mg, 0.260 mmol, 0.06 equiv) and toluene (15 mL). The resulting mixture was stirred for 5 h at 50° C. in an oil bath and then concentrated under vacuum. The residue was dissolved in ethyl acetate and washed with water (10 mL) and concentrated under vacuum. The residue was applied onto a silica gel column with ethyl acetate/petroleum ether (5:95) to yield 4,4,5,5-tetramethyl-2-(1-phenylethenyl)-1,3,2-dioxaborolane as brown oil.

### Step 2. Ethyl 3-(2-chloro-4-[[4-(1-phenylethenyl)-5,6-dihydro-2H-pyran-3-yl]methoxy]phenyl)propanoate

Into a 50-mL round-bottom flask was added ethyl 3-[4-[(4-bromo-5,6-dihydro-2H-pyran-3-yl)methoxy]-2-chlorophenyl]propanoate (100 mg, 0.250 mmol, 1.00 equiv), 4,4,5,5-tetramethyl-2-(1-phenylethenyl)-1,3,2-dioxaborolane (68.7 mg, 0.300 mmol, 1.21 equiv), Pd(dppf)Cl₂ (18.2 mg, 0.020 mmol, 0.10 equiv), K₃PO₄ (105.4 mg, 0.500 mmol, 2.00 equiv), 1,4-dioxane (4 mL) and water (2 mL). The resulting mixture was stirred overnight at 80° C., then diluted with water (5 mL) and extracted with ethyl acetate (3×5 mL). The organic layers were combined and washed with brine (3×5 mL). The mixture was dried over anhydrous sodium sulfate and concentrated under vacuum. The residue was applied onto a silica gel column with ethyl acetate/petroleum ether (1:5) to yield ethyl 3-(2-chloro-4-[[4-(1-phenylethenyl)-5,6-dihydro-2H-pyran-3-yl]methoxy]phenyl)propanoate as colorless oil.

### Step 3. 3-(2-chloro-4-[[4-(1-phenylethenyl)-5,6-dihydro-2H-pyran-3-yl]methoxy]phenyl)propanoic acid

Into a 50-mL round-bottom flask was added ethyl 3-(2-chloro-4-[[4-(1-phenylethenyl)-5,6-dihydro-2H-pyran-3-yl]methoxy]phenyl)propanoate (45 mg, 0.110 mmol, 1.00 equiv), tetrahydrofuran (1 mL), LiOH (45 mg, 1.880 mmol, 17.83 equiv) and water (1 mL) and the reaction was stirred overnight at 20° C. The resulting mixture was diluted with ethyl acetate (5 mL) and the pH adjusted to 5-6 with 2N hydrochloric acid. The resulting solution was extracted with ethyl acetate (3×5 mL) and the organic layers combined and washed with brine (3×5 mL). The resulting mixture was concentrated under vacuum. The residue was purified by reversed-phase HPLC on a SunFire C18 column (19 mm×100 mm, 5 μM) with a linear gradient of 30-78% acetonitrile in water (0.05% TFA) in 10 mins to yield 3-(2-chloro-4-[[4-(1-phenylethenyl)-5,6-dihydro-2H-pyran-3-yl]methoxy] phenyl)propanoic acid as colorless oil.

¹H NMR (300 MHz, CD₃OD) δ: 7.25-7.41 (m, 5H), 7.15 (d, J=8.4 Hz, 1H), 6.86 (s, 1H), 6.72 (d, J=8.4 Hz, 1H), 5.62 (s, 1H), 5.13 (s, 1H), 4.52 (s, 2H), 4.35 (s, 2H), 3.83 (t, J=8.4 Hz, 2H), 2.91 (t, J=7.8 Hz, 2H), 2.56 (t, J=7.8 Hz, 2H), 2.17-2.22 (m, 2H). Mass spectrum (ESI, m/z): Calculated for C₂₃H₂₃ClO₄, 397.1 [M−H], Measured 397.1.

The following compounds were similarly prepared according to the procedure as described in Example 92 above, selecting and substituting suitably substituted reactants, as would be readily recognized by those skilled in the art.

### Example 93

### 3-[4-[[4-[(4-chlorophenyl)methyl]-3,6-dihydro-2H-pyran-5-yl]methoxy]-3,5-difluoro-phenyl]propanoic acid

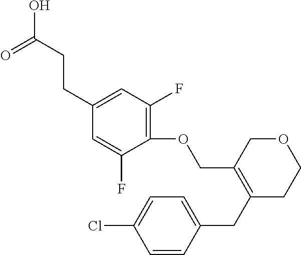 [SMILES: O=C(O)CCc1cc(F)c(OCC2=C(Cc3ccc(Cl)cc3)CCOC2)c(F)c1]

¹H NMR (300 MHz, CD₃OD) δ:7.24 (d, J=8.4 Hz, 2H), 7.10 (d, J=8.4 Hz, 2H), 6.86-6.94 (m, 2H), 4.68 (s, 2H), 4.33 (s, 2H), 3.72 (t, J=8.4 Hz, 2H), 3.44 (s, 2H), 2.88 (t, J=7.8 Hz, 2H), 2.61 (t, J=7.8 Hz, 2H), 1.95-1.99 (m, 2H). ¹⁹F NMR (300 MHz, CD₃OD) δ: −129.94. Mass spectrum (ESI, m/z): Calculated for C₂₂H₂₁ClF₂O₄, 421.1 [M−H], Measured 421.0.

### Example 94

### 3-[4-[(4-isopentyl-3,6-dihydro-2H-pyran-5-yl)methoxy]-2-(trifluoromethyl)phenyl]propanoic acid

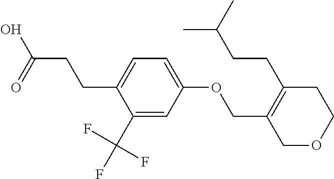 [SMILES: CC(C)CCC1=C(COc2ccc(CCC(=O)O)c(C(F)(F)F)c2)COCC1]

¹H NMR (CHLOROFORM-d) δ: 7.26-7.30 (m, 1H), 7.17 (d, J=3.0 Hz, 1H), 7.01 (dd, J=8.6, 2.5 Hz, 1H), 4.48 (s, 2H), 4.20 (s, 2H), 3.82 (t, J=5.6 Hz, 2H), 3.07 (br t, J=7.8 Hz, 2H), 2.66 (t, J=8.1 Hz, 2H), 2.06-2.21 (m, 3H), 1.54 (dquin, J=13.5, 6.6 Hz, 1H), 1.24-1.35 (m, 2H), 0.89 (d, J=6.6 Hz, 5H). Mass spectrum (ESI, m/z): Calculated for C₂₁H₂₇F₃O₄, 423.1 [M+Na], Measured 423.3.

### Example 95

### 3-[3,5-difluoro-4-[[4-(1-phenylvinyl)-3,6-dihydro-2H-pyran-5-yl]methoxy]phenyl]propanoic acid

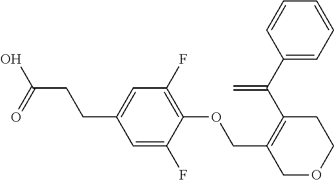 [SMILES: C=C(C1=C(COc2c(F)cc(CCC(=O)O)cc2F)COCC1)c1ccccc1]

¹H NMR (300 MHz, CD₃OD) δ: 7.72-7.28 (s, 5H), 6.82 (d, J=9.0 Hz, 2H), 5.51 (s, 1H), 4.90 (s, 1H), 4.61 (s, 2H), 4.45 (s, 2H), 3.8 (t, J=5.6 Hz, 2H), 2.85 (t, J=7.6 Hz, 2H), 2.58 (t, J=7.6 Hz, 2H), 2.14 (brs, 2H). ¹⁹F NMR (300 MHz, CD₃OD) δ: −129.84. Mass spectrum (ESI, m/z): Calculated for C_{23.}H₂₂F₂O₄, 399.1 [M−H], Measured 399.1.

### Example 96

### 3-[4-[(4-cyclobutyl-3,6-dihydro-2H-pyran-5-yl)methoxy]-2,3-dimethyl-phenyl]propanoic acid

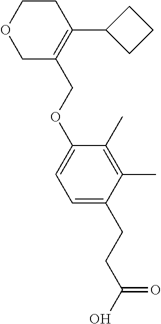 [SMILES: Cc1c(CCC(=O)O)ccc(OCC2=C(C3CCC3)CCOC2)c1C]

¹H NMR (CHLOROFORM-d) δ: 6.97 (d, J=8.1 Hz, 1H), 6.66 (d, J=8.1 Hz, 1H), 4.40 (s, 2H), 4.22 (br s, 2H), 3.83 (t, J=5.6 Hz, 2H), 3.45 (quin, J=8.8 Hz, 1H), 2.86-2.98 (m, 2H), 2.53-2.66 (m, 2H), 2.22 (s, 5H), 2.15 (s, 3H), 1.94-2.11 (m, 4H), 1.81-1.94 (m, 1H), 1.66-1.78 (m, 1H). Mass spectrum (ESI, m/z): Calculated for C₂₁H₂₈O₄, 367.1 [M+Na], Measured 367.3.

### Example 97

### 3-[2-chloro-4-[(4-cyclobutyl-3,6-dihydro-2H-pyran-5-yl)methoxy]phenyl]propanoic acid

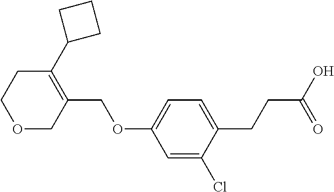 [SMILES: O=C(O)CCc1ccc(OCC2=C(C3CCC3)CCOC2)cc1Cl]

¹H NMR (CHLOROFORM-d) δ: 7.15 (d, J=8.1 Hz, 1H), 6.92 (d, J=2.5 Hz, 1H), 6.75 (dd, J=8.3, 2.3 Hz, 1H), 4.39 (s, 2H), 4.18 (br s, 2H), 3.83 (br t, J=5.3 Hz, 2H), 3.44 (quin, J=8.8 Hz, 1H), 3.00 (br t, J=7.6 Hz, 2H), 2.68 (br t, J=7.3 Hz, 2H), 2.24 (br s, 2H), 1.96-2.15 (m, 4H), 1.81-1.96 (m, 1H), 1.66-1.80 (m, 1H). Mass spectrum (ESI, m/z): Calculated for C₁₉H₂₃ClO₄, 373.1 [M+Na], Measured 373.0.

### Example 98

### 3-[2-chloro-4-[(4-cyclopentyl-3,6-dihydro-2H-pyran-5-yl)methoxy]phenyl]propanoic acid

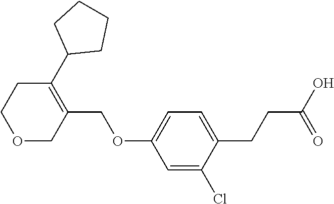 [SMILES: O=C(O)CCc1ccc(OCC2=C(C3CCCC3)CCOC2)cc1Cl]

¹H NMR (CHLOROFORM-d) δ: 7.16 (d, J=8.6 Hz, 1H), 6.93 (d, J=2.5 Hz, 1H), 6.76 (dd, J=8.6, 2.5 Hz, 1H), 4.44 (s, 3H), 4.20 (s, 2H), 3.81 (t, J=5.3 Hz, 2H), 2.87-3.05 (m, 3H), 2.67 (t, J=7.6 Hz, 2H), 2.13 (br s, 2H), 1.50-1.76 (m, 6H), 1.43 (td, J=11.2, 4.3 Hz, 2H). Mass spectrum (ESI, m/z): Calculated for C₂₀H₂₅ClO₄, 387.1 [M+Na], Measured 387.0.

### Example 99

### 3-[4-[(4-cyclohexyl-3,6-dihydro-2H-pyran-5-yl)methoxy]-2-(trifluoromethyl)phenyl]propanoic acid

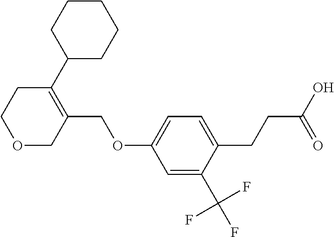 [SMILES: O=C(O)CCc1ccc(OCC2=C(C3CCCCC3)CCOC2)cc1C(F)(F)F]

### Step 1. Methyl 4-cyclohexyl-5,6-dihydro-2H-pyran-3-carboxylate

Into a 100-mL round-bottom flask purged and maintained with an inert atmosphere of nitrogen was added methyl 4-[(trifluoromethane)sulfonyloxy]-5,6-dihydro-2H-pyran-3-carboxylate (200 mg, 0.69-mmol, 1.00 equiv), Pd(PPh₃)₄ (398 mg, 0.340 mmol, 0.50 equiv), tetrahydrofuran (5 mL). This was followed by the addition of (cyclohexyl)zinc bromide solution (0.5 M in THF, 2.7 mL, 2.00 equiv) dropwise with stirring at 0° C. The resulting solution was stirred overnight at 50° C. in an oil bath and then quenched by the addition of water (20 mL). The resulting mixture was extracted with ethyl acetate (3×30 mL) and the organic layers combined and washed with brine (1×30 mL). The resulting mixture was concentrated under vacuum and the residue was applied onto a silica gel column with ethyl acetate/petroleum ether (1:4) to yield methyl 4-cyclohexyl-5,6-dihydro-2H-pyran-3-carboxylate as light yellow oil. Mass spectrum (EI, m/z): Calculated for C₁₃H₂₀O₃, 224.1 [M], Measured 224.1.

### Step 2. (4-cyclohexyl-5,6-dihydro-2H-pyran-3-yl)methanol

To a solution of methyl 4-cyclohexyl-5,6-dihydro-2H-pyran-3-carboxylate (100 mg, 0.450 mmol, 1.00 equiv) in dichloromethane (4 mL) at −78° C. was added DIBAL (1M in toluene, 0.9 mL, 2.00 equiv) dropwise with stirring. The resulting solution was stirred for 2 h at −78° C. and then quenched by the addition of methanol (10 mL). The resulting mixture was concentrated under vacuum and the residue was applied onto a silica gel column with ethyl acetate/petroleum ether (1:2) to yield (4-cyclohexyl-5,6-dihydro-2H-pyran-3-yl)methanol as colorless oil. Mass spectrum (EI, m/z): Calculated for C₁₂H₂₀O₂, 178.1 [M-18], Measured 178.1.

### Step 3. Ethyl 3-[4-[(4-cyclohexyl-5,6-dihydro-2H-pyran-3-yl)methoxy]-2-(trifluoromethyl)phenyl]propanoate

Into a 50-mL round-bottom flask purged and maintained with an inert atmosphere of nitrogen was added (4-cyclohexyl-5,6-dihydro-2H-pyran-3-yl)methanol (40 mg, 0.200 mmol, 1.00 equiv), ethyl 3-[4-hydroxy-2-(trifluoromethyl)phenyl]propanoate (64 mg, 0.240 mmol, 1.20 equiv), ADDP (127 mg, 0.510 mmol, 2.50 equiv), n-Bu₃P (62 mg, 0.310 mmol, 1.50 equiv), and toluene (5 mL). The resulting solution was stirred overnight at 60° C. in an oil bath and then concentrated under vacuum. The residue was applied onto a silica gel column with ethyl acetate/petroleum ether (1:4) to yield ethyl 3-[4-[(4-cyclohexyl-5,6-dihydro-2H-pyran-3-yl)methoxy]-2-(trifluoromethyl)phenyl]propanoate as light yellow oil.

### Step 4. ethyl 3-[4-[(4-cyclohexyl-5,6-dihydro-2H-pyran-3-yl)methoxy]-2-(trifluoromethyl)phenyl]propanoate

Into a 50-mL round-bottom flask was added ethyl 3-[4-[(4-cyclohexyl-5,6-dihydro-2H-pyran-3-yl)methoxy]-2-(trifluoromethyl)phenyl]propanoate (40 mg, 0.090 mmol, 1.00 equiv), tetrahydrofuran (2 mL), LiOH (11 mg, 0.460 mmol, 5.00 equiv) and water (1 mL) and the reaction was stirred for 2 h at 25° C. The pH was adjusted to 6 with 2N hydrochloric acid and the resulting solution was extracted with ethyl acetate (3×50 mL) and the organic layers combined and washed with brine (1×50 mL) and concentrated. The residue was purified by reversed-phase HPLC on a SunFire C18 column (19 mm×100 mm, 5 μM) with a linear gradient of 60-90% acetonitrile in water (0.05% TFA) in 10 mins to yield ethyl 3-[4-[(4-cyclohexyl-5,6-dihydro-2H-pyran-3-yl)methoxy]-2-(trifluoromethyl)phenyl]propanoate as light yellow oil.

¹H NMR (400 MHz, CD₃OD) δ: 7.39 (d, J=8.8 Hz, 1H), 7.20 (s, 1H), 7.14 (dd, J₁=2.4 Hz, J₂=8.4 Hz, 1H), 4.57 (s, 2H), 4.17 (s, 2H), 3.77 (t, J=5.6 Hz, 2H), 3.04 (t, J=7.6 Hz, 2H), 2.57-2.63 (m, 3H), 2.10-2.15 (m, 2H), 1.68-1.80 (m, 3H), 1.44-1.54 (m, 2H), 1.33-1.40 (m, 5H). ¹⁹F NMR (400 MHz, CD₃OD) δ: −61.35. Mass spectrum (ESI, m/z): Calculated for C₂₂H₂₇F₃O₄, 411.2 [M−H], Measured 411.1.

The following compounds were similarly prepared according to the procedure as described in Example 99 above, selecting and substituting suitably substituted reactants, as would be readily recognized by those skilled in the art.

### Example 100

### 3-[4-[(4-cyclopentyl-3,6-dihydro-2H-pyran-5-yl)methoxy]-2-(trifluoromethyl)phenyl]propanoic acid

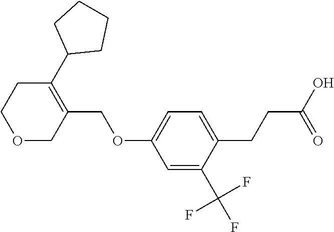 [SMILES: O=C(O)CCc1ccc(OCC2=C(C3CCCC3)CCOC2)cc1C(F)(F)F]

¹H NMR (300 MHz, CD₃OD) δ:7.34 (d, J=8.4 Hz, 1H), 7.07-7.15 (m, 2H), 4.54 (s, 2H), 4.14 (s, 2H), 3.74 (t, J=5.4 Hz, 2H), 2.96-3.08 (m, 3H), 2.52 (t, J=8.1 Hz, 2H), 2.05-2.10 (m, 2H), 1.55-1.68 (m, 6H), 1.43-1.51 (m, 2H). ¹⁹F NMR (300 MHz, CD₃OD) δ: −61.34. Mass spectrum (ESI, m/z): Calculated for C₂₁H₂₅F₃O₄, 397.2 [M−H], Measured 397.2.

### Example 101

### 3-[2-chloro-4-[(4-isobutyl-3,6-dihydro-2H-pyran-5-yl)methoxy]phenyl]propanoic acid

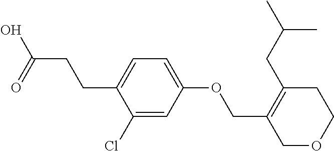 [SMILES: CC(C)CC1=C(COc2ccc(CCC(=O)O)c(Cl)c2)COCC1]

¹H NMR (400 MHz, CD₃OD) δ: 7.24 (d, J=8.4 Hz, 1H), 6.97 (s, 1H), 6.83 (dd, J₁=2.4 Hz, J₂=8.4 Hz, 1H), 4.51 (s, 2H), 4.20 (s, 2H), 3.79 (t, J=5.6 Hz, 2H), 2.80 (t, J=7.6 Hz, 2H), 2.59 (t, J=7.6 Hz, 2H), 2.15-2.20 (m, 2H), 2.09 (d, J=7.6 Hz, 2H), 1.80-1.87 (m, 1H), 0.92 (d, J=6.4 Hz, 6H). Mass spectrum (ESI, m/z): Calculated for C₁₉H₂₅ClO₄, 351.1 [M−H], Measured 351.1.

### Example 102

### 3-[3,5-difluoro-4-[(4-isobutyl-3,6-dihydro-2H-pyran-5-yl)methoxy]phenyl]propanoic acid

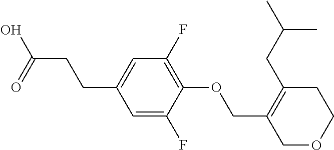 [SMILES: CC(C)CC1=C(COc2c(F)cc(CCC(=O)O)cc2F)COCC1]

¹H NMR (300 MHz, CD₃OD) δ: 6.81-6.89 (m, 2H), 4.52 (s, 2H), 4.25 (s, 2H), 3.73 (t, J=5.4 Hz, 2H), 2.83 (t, J=7.6 Hz, 2H), 2.55 (t, J=7.4 Hz, 2H), 2.07-2.09 (m, 2H), 1.94 (d, J=7.5 Hz, 2H), 1.68-1.77 (m, 1H), 0.81 (d, J=6.6 Hz, 6H). ¹⁹F NMR (300 MHz, CD₃OD) δ: −130.07. Mass spectrum (ESI, m/z): Calculated for C₁₉H₂₄F₂O₄, 353.2 [M−H], Measured 353.2.

### Example 103

### 3-[4-[(4-sec-butyl-3,6-dihydro-2H-pyran-5-yl)methoxy]-2-(trifluoromethyl)phenyl]propanoic acid

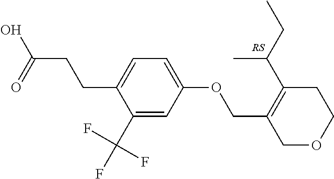 [SMILES: CCC(C)C1=C(COc2ccc(CCC(=O)O)c(C(F)(F)F)c2)COCC1]

### Step 1. Methyl 4-(butan-2-yl)-5,6-dihydro-2H-pyran-3-carboxylate

Into a 50-mL round-bottom flask purged and maintained with an inert atmosphere of nitrogen was added methyl 4-[(trifluoromethane)sulfonyloxy]-5,6-dihydro-2H-pyran-3-carboxylate (300 mg, 1.030 mmol, 1.00 equiv), Pd(OAc)₂ (25.4 mg, 0.110 mmol, 0.10 equiv), RuPhos (97.1 mg 0.208 mmol, 0.20 equiv), and tetrahydrofuran (5 mL). This was followed by the addition of (butan-2-yl)zinc bromide solution (0.624 mL, 1.250 mmol, 1.20 equiv) dropwise with stirring at 0° C. The resulting solution was stirred overnight at 20° C. and then quenched by the addition of water (5 mL). The resulting solution was extracted with ethyl acetate (2×10 mL) and the organic layers combined and washed with brine. The mixture was dried over anhydrous sodium sulfate and concentrated under vacuum. The residue was applied onto a silica gel column with ethyl acetate/petroleum ether (8:92) to yield methyl 4-(butan-2-yl)-5,6-dihydro-2H-pyran-3-carboxylate as colorless oil and recovered methyl 4-[(trifluoromethane)sulfonyloxy]-5,6-dihydro-2H-pyran-3-carboxylate as colorless oil. ¹H NMR (300 MHz, CDCl₃) δ: 4.27-4.38 (m, 2H), 3.76-3.78 (m, 2H), 3.75 (s, 3H), 3.51-3.53 (m, 1H), 2.52-3.56 (m, 1H), 2.17-2.30 (m, 2H), 1.33-1.48 (m, 3H), 1.03 (d, J=6.8 Hz, 3H), 0.87 (t, J=7.6 Hz, 3H).

### Step 2. [4-(butan-2-yl)-5,6-dihydro-2H-pyran-3-yl]methanol

To a solution of methyl 4-(butan-2-yl)-5,6-dihydro-2H-pyran-3-carboxylate (80 mg, 0.400 mmol, 1.00 equiv) in tetrahydrofuran (3 mL) at 0° C. was added LiAlH₄ (30.7 mg, 0.810 mmol, 2.00 equiv). The resulting mixture was stirred for 30 min at 0° C. in a water/ice bath. The reaction was then quenched by the addition of sodium sulfate.10H₂O. The solids were filtered out. The resulting mixture was concentrated under vacuum. The residue was applied onto a silica gel column with ethyl acetate/petroleum ether (25:75) to yield [4-(butan-2-yl)-5,6-dihydro-2H-pyran-3-yl]methanol as colorless oil.

### Step 3. 3-(4-((4-sec-butyl-5,6-dihydro-2H-pyran-3-yl)methoxy)-2-(trifluoromethyl)phenyl)propanoic acid

Into a 25-mL round-bottom flask purged and maintained with an inert atmosphere of nitrogen, was added [4-(butan-2-yl)-5,6-dihydro-2H-pyran-3-yl]methanol (50 mg, 0.290 mmol, 1.00 equiv), ethyl 3-[4-hydroxy-2-(trifluoromethyl)phenyl]propanoate (92.5 mg, 0.350 mmol, 1.20 equiv), ADDP (185.3 mg, 0.740 mmol, 2.52 equiv), n-Bu₃P (89.1 mg, 0.441 mmol, 1.50 equiv), and toluene (3 mL). The resulting solution was stirred overnight at 70° C. in an oil bath and the resulting mixture was concentrated under vacuum. The residue was applied onto a silica gel column with ethyl acetate/petroleum ether (1:4) to yield ethyl 3-(4-[[4-(butan-2-yl)-5,6-dihydro-2H-pyran-3-yl]methoxy]-2-(trifluoromethyl)phenyl)propanoate as colorless oil.

### Step 4. 3-(4-[[4-(butan-2-yl)-5,6-dihydro-2H-pyran-3-yl]methoxy]-2-(trifluoromethyl)phenyl)propanoic acid

Into a 25-mL round-bottom flask, was placed ethyl 3-(4-[[4-(butan-2-yl)-5,6-dihydro-2H-pyran-3-yl]methoxy]-2-(trifluoromethyl)phenyl)propanoate (60 mg, 0.140 mmol, 1.00 equiv), tetrahydrofuran (1.5 mL), LiOH (60 mg, 2.510 mmol, 17.31 equiv) and water (1.5 mL). The resulting solution was stirred overnight at 20° C. and then the pH of the solution was adjusted to 4 with 2M hydrochloric acid and the resulting solution was extracted with ethyl acetate (3×5 mL). The organic layers were combined and washed with brine solution (2×5 mL) and concentrated under vacuum. The residue was purified by reversed-phase HPLC on a SunFire C18 column (19 mm×100 mm, 5 μM) with a linear gradient of 65-95% acetonitrile in water (0.05% TFA) in 9 mins to yield 3-(4-[[4-(butan-2-yl)-5,6-dihydro-2H-pyran-3-yl]methoxy]-2-(trifluoromethyl)phenyl)propanoic acid as light yellow oil.

¹H NMR (400 MHz, CD₃OD) δ: 7.27 (d, J=8.8 Hz, 1H), 6.97-7.14 (m, 2H), 4.41-4.50 (m, 2H), 4.01-4.09 (m, 2H), 3.63-3.72 (m, 2H), 2.92 (t, J=8.0 Hz, 2H), 2.57-2.65 (m, 1H), 2.46 (t, J=8.0 Hz, 2H), 1.97-2.04 (m, 2H), 1.26-1.33 (m, 2H), 0.91 (d, J=6.8 Hz, 3H), 0.74 (t, J=7.4 Hz, 3H). ¹⁹F NMR (400 MHz, CD₃OD) δ: −61.35. Mass spectrum (ESI, m/z): Calculated for C₂₀H₂₅F₃O₄, 385.2 [M−H], Measured 385.1.

The following compounds were similarly prepared according to the procedure as described in Example 103 above, selecting and substituting suitably substituted reactants, as would be readily recognized by those skilled in the art.

### Example 104

### 3-[4-[(4-isopropyl-3,6-dihydro-2H-pyran-5-yl)methoxy]-2-(trifluoromethyl)phenyl]propanoic acid

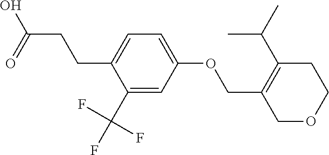 [SMILES: CC(C)C1=C(COc2ccc(CCC(=O)O)c(C(F)(F)F)c2)COCC1]

¹H NMR (300 MHz, CD₃OD) δ: 7.36 (d, J=8.4 Hz, 1H), 7.02-7.15 (m, 2H), 4.51 (s, 2H), 4.13 (s, 2H), 3.74 (t, J=5.5 Hz, 2H), 2.89-3.05 (m, 3H), 2.32-2.46 (m, 2H), 2.04-2.11 (m, 2H), 0.99 (d, J=6.9 Hz, 6H). ¹⁹F NMR (300 MHz, CD₃OD) δ: −61.24. Mass spectrum (ESI, m/z): Calculated for C₁₉H₂₃F₃O₄, 371.2 [M−H], Measured 371.1.

### Example 105

### 3-[4-[(4-cyclopentyl-3,6-dihydro-2H-pyran-5-yl)methoxy]-3,5-difluoro-phenyl]propanoic acid

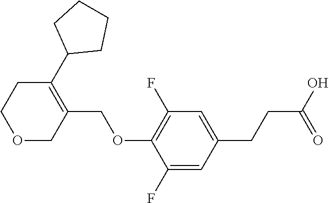 [SMILES: O=C(O)CCc1cc(F)c(OCC2=C(C3CCCC3)CCOC2)c(F)c1]

¹H NMR (CHLOROFORM-d) δ: 6.68-6.84 (m, 2H), 4.55 (s, 2H), 4.30 (t, J=2.0 Hz, 2H), 3.79 (t, J=5.6 Hz, 2H), 2.91-3.02 (m, 1H), 2.83-2.91 (m, 2H), 2.59-2.70 (m, 2H), 2.01-2.13 (m, 2H), 1.40-1.70 (m, 6H), 1.20-1.39 (m, 2H). Mass spectrum (ESI, m/z): Calculated for C₂₀H₂₄F₂O₄, 389.1 [M+Na], Measured 389.3.

### Example 106

### 3-[2-chloro-4-[(4-cyclopent-2-en-1-yl-3,6-dihydro-2H-pyran-5-yl)methoxy]phenyl]propanoic acid

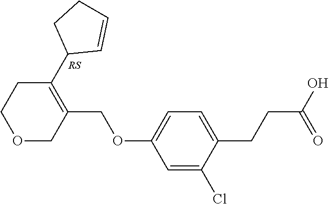 [SMILES: O=C(O)CCc1ccc(OCC2=C(C3C=CCC3)CCOC2)cc1Cl]

¹H NMR (CHLOROFORM-d) δ: 7.16 (d, J=8.1 Hz, 1H), 6.94 (d, J=2.5 Hz, 1H), 6.76 (dd, J=8.6, 2.5 Hz, 1H), 5.83-5.89 (m, 1H), 5.52 (dd, J=5.6, 2.5 Hz, 1H), 4.42-4.55 (m, 2H), 4.20 (d, J=1.5 Hz, 2H), 3.86-3.95 (m, 1H), 3.70-3.84 (m, 2H), 3.00 (t, J=7.6 Hz, 2H), 2.67 (t, J=7.8 Hz, 2H), 2.28-2.46 (m, 2H), 2.00-2.14 (m, 3H), 1.64 (ddt, J=13.3, 9.1, 6.8 Hz, 1H). Mass spectrum (ESI, m/z): Calculated for C₂₀H₂₃ClO₄, 385.1 [M+Na], Measured 385.3.

### Example 107

### 3-[4-[(4-cyclopentyl-3,6-dihydro-2H-pyran-5-yl)methoxy]phenyl]propanoic acid

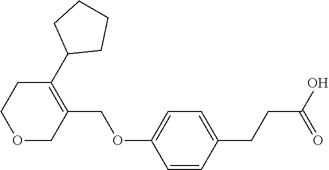 [SMILES: O=C(O)CCc1ccc(OCC2=C(C3CCCC3)CCOC2)cc1]

¹H NMR (CHLOROFORM-d) δ: 7.13 (d, J=8.6 Hz, 2H), 6.86 (d, J=8.6 Hz, 2H), 4.45 (s, 2H), 4.22 (s, 2H), 3.80 (t, J=5.6 Hz, 2H), 2.93-3.06 (m, 1H), 2.87-2.93 (m, 2H), 2.60-2.69 (m, 2H), 2.13 (br s, 2H), 1.49-1.74 (m, 6H), 1.43 (td, J=11.4, 4.0 Hz, 2H). Mass spectrum (ESI, m/z): Calculated for C₂₀H₂₆O₄, 353.1 [M+Na], Measured 353.2.

### Example 108

### 3-[4-[(4-cyclopent-2-en-1-yl-3,6-dihydro-2H-pyran-5-yl)methoxy]-2-(trifluoromethyl)phenyl]propanoic acid

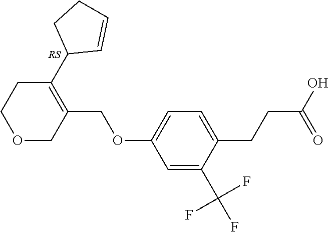 [SMILES: O=C(O)CCc1ccc(OCC2=C(C3C=CCC3)CCOC2)cc1C(F)(F)F]

¹H NMR (CHLOROFORM-d) δ: 7.25-7.30 (m, 1H), 7.17 (d, J=2.5 Hz, 1H), 7.02 (dd, J=8.6, 2.5 Hz, 1H), 5.85-5.91 (m, 1H), 5.51 (dq, J=5.9, 2.1 Hz, 1H), 4.45-4.61 (m, 2H), 4.23 (br d, J=1.5 Hz, 2H), 3.91 (br d, J=2.5 Hz, 1H), 3.74-3.86 (m, 2H), 3.07 (t, J=8.1 Hz, 2H), 2.60-2.69 (m, 2H), 2.22-2.54 (m, 2H), 2.03-2.18 (m, 3H), 1.64 (ddt, J=13.4, 9.3, 6.6 Hz, 1H). Mass spectrum (ESI, m/z): Calculated for C₂₁H₂₃F₃O₄, 419.1 [M+Na], Measured 419.3.

### Example 109

### 3-[4-[(4-cyclopent-2-en-1-yl-3,6-dihydro-2H-pyran-5-yl)methoxy]phenyl]propanoic acid

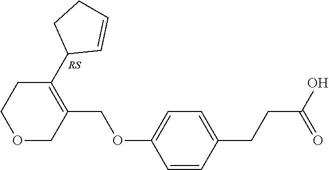 [SMILES: O=C(O)CCc1ccc(OCC2=C(C3C=CCC3)CCOC2)cc1]

¹H NMR (CHLOROFORM-d) δ: 7.12 (d, J=8.6 Hz, 2H), 6.85 (d, J=8.6 Hz, 2H), 5.80-5.91 (m, 1H), 5.51 (dd, J=5.6, 2.0 Hz, 1H), 4.41-4.57 (m, 2H), 4.22 (br d, J=2.0 Hz, 2H), 3.92 (br dd, J=4.5, 2.0 Hz, 1H), 3.70-3.86 (m, 2H), 2.84-2.97 (m, 2H), 2.60-2.71 (m, 2H), 2.20-2.53 (m, 2H), 1.99-2.16 (m, 3H), 1.63 (ddt, J=13.4, 9.3, 6.6 Hz, 1H).). Mass spectrum (ESI, m/z): Calculated for C₂₀H₂₄O₄, 351.1 [M+Na], Measured 351.3.

### Example 110

### 3-(4-((4-(4-chlorophenyl)-2,5-dihydrofuran-3-yl)methoxy)-2,3-dimethylphenyl)propanoic acid

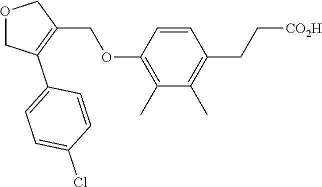 [SMILES: Cc1c(CCC(=O)O)ccc(OCC2=C(c3ccc(Cl)cc3)COC2)c1C]

### Step 1: methyl 4-(((trifluoromethyl)sulfonyl)oxy)-2,5-dihydrofuran-3-carboxylate

To a solution of DIEA (860 μL, 4.99 mmol) in dry DCM (20 mL) cooled to −78° C. under Argon was added a solution of methyl 4-oxotetrahydrofuran-3-carboxylate (628 mg, 4.63 mmol in 20 mL DCM) dropwise. The reaction mixture was stirred @ −78° C. for 30 minutes post addition and then treated dropwise with neat trifluoromethanesulfonic anhydride (820 μL, 4.98 mmol). After stirring the reaction mixture for 2 h @ 0° C., the reaction mixture was quenched by the cautious addition of cold water (11 mL). The reaction mixture was warmed to ambient temperature and diluted with water and diethyl ether. The aqueous layer was extracted with diethyl ether and the combined organic layers washed with sat. NaHCO₃ and brine, then dried over MgSO₄. The resulting residue was concentrated in vacuo followed by flash chromatography (SiO₂, 0-50% DCM/heptane) to yield a pale yellow oil.

### Step 2: methyl 4-(4-chlorophenyl)-2,5-dihydrofuran-3-carboxylate

A suspension of anhydrous potassium phosphate (1.07 g, 5.04 mmol), tetrakis(triphenylphosphine) palladium (143 mg, 0.124 mmol), 4-chlorophenylboronic acid (268 mg, 1.714 mmol) and methyl 4-(((trifluoromethyl)sulfonyl)oxy)-2,5-dihydrofuran-3-carboxylate (390 mg, 1.412 mmol) in 1,4-dioxane (20 mL) was sparged with a stream of argon for 20 minutes. The reaction was then placed under an Argon atmosphere and heated to 90° C. for 16 hours, before cooling to ambient temperature. The mixture was diluted with ether and filtered through a plug of CELITE®. The filtrate was concentrated in vacuo and purified by flash chromatography (SiO₂, 0-100% DCM/heptane) to yield a residue.

### Step 3: (4-(4-chlorophenyl)-2,5-dihydrofuran-3-yl)methanol

A solution of methyl 4-(4-chlorophenyl)-2,5-dihydrofuran-3-carboxylate (268 mg, 1.123 mmol) in dry toluene (15 mL) was cooled to −78° C. under an Argon atmosphere. The reaction mixture was treated dropwise with DiBA-H (2.5 mL, 2.5 mmol in toluene) and stirred, allowing the reaction mixture to reach ambient temperature over 2 hours. After stirred one additional hour, the mixture was cooled to ˜−10° C. and quenched by the dropwise addition of sat. Rochelle's salt (12 mL). The aqueous layer was extracted with diethyl ether and the combined organic layers washed with sat. NaHCO₃ and brine, then dried over MgSO₄. The resulting residue was concentrated in vacuo followed by flash chromatography (SiO₂, diethyl ether) to yield a colorless crystalline solid.

### Step 4: ethyl 3-(4-((4-(4-chlorophenyl)-2,5-dihydrofuran-3-yl)methoxy)-2,3-dimethylphenyl)propanoate

A solution of (4-(4-chlorophenyl)-2,5-dihydrofuran-3-yl)methanol (105 mg, 0.498 mmol), DEAD (110 μL, 0.701 mmol) and methyl 3-(4-hydroxy-3-methylphenyl)propanoate (121 mg, 0.548 mmol) in toluene (7 mL) was placed under an argon atmosphere, cooled to ˜0° C. and treated with triphenylphosphine (183 mg, 0.698 mmol). The reaction was stirred warming gradually to ambient temperature at for ˜18 hours and then concentrated in vacuo. The residue was triturated with diethyl ether, the resulting white precipitate was filtered off and the filtrated concentrated and purified by flash chromatography (SiO₂, 6:7 DCM:heptane) to yield a residue.

### Step 5: 3-(4-((4-(4-chlorophenyl)-2,5-dihydrofuran-3-yl)methoxy)-2,3-dimethylphenyl)propanoic acid

A solution of ethyl 3-(4-((4-(4-chlorophenyl)-2,5-dihydrofuran-3-yl)methoxy)-2,3-dimethylphenyl)propanoate (123 mg, 0.296 mmol) was dissolved in THF (7 mL), water (3 mL) and methanol (3 mL) before being treated with a solution of KOH (0.600 mL, 3 mmol, SM). After stirring at ambient temperature overnight, the reaction mixture was adjusted to pH 4 with 1N HCl, then extracted with ethyl acetate. The organic extracts were dried (MgSO₄) and concentrated in vacuo to yield the title compound.

¹H NMR (CHLOROFORM-d) δ: 7.32-7.39 (m, 2H), 7.18 (d, J=8.6 Hz, 2H), 6.90-6.97 (m, 1H), 6.52-6.63 (m, 1H), 4.95-5.07 (m, 4H), 4.71 (s, 2H), 2.87-2.98 (m, 2H), 2.53-2.64 (m, 2H), 2.22 (s, 3H), 2.15 (s, 3H)

### Example 111

### 3-(2,3-dimethyl-4-((4-phenyl-2,5-dihydrofuran-3-yl)methoxy)phenyl)propanoic acid

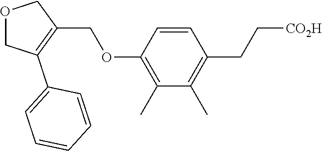 [SMILES: Cc1c(CCC(=O)O)ccc(OCC2=C(c3ccccc3)COC2)c1C]

The title compound was prepared as describe in General Synthesis Scheme 1, selecting and substituting suitable starting materials and reagents as would be readily recognized by those skilled in the art.

¹H NMR (CHLOROFORM-d) δ: 7.30-7.41 (m, 3H), 7.21-7.27 (m, 2H), 6.89-6.96 (m, 1H), 6.54-6.62 (m, 1H), 4.97-5.12 (m, 4H), 4.76 (s, 2H), 2.87-2.96 (m, 2H), 2.54-2.62 (m, 2H), 2.22 (s, 3H), 2.12-2.19 (m, 3H).

### Example 112

### 3-(3-methyl-4-((4-phenyl-2,5-dihydrofuran-3-yl)methoxy)phenyl)propanoic acid

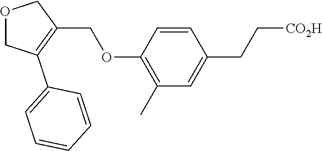 [SMILES: Cc1cc(CCC(=O)O)ccc1OCC1=C(c2ccccc2)COC1]

The title compound was prepared as describe in General Synthesis Scheme 1, selecting and substituting suitable starting materials and reagents as would be readily recognized by those skilled in the art.

¹H NMR (CHLOROFORM-d) δ: 7.29-7.44 (m, 3H), 7.26 (s, 2H), 6.86-7.04 (m, 2H), 6.60-6.68 (m, 1H), 4.97-5.12 (m, 4H), 4.77 (s, 2H), 2.81-2.91 (m, 2H), 2.56-2.69 (m, 2H), 2.19 (s, 3H).

### Example 113

### 3-(4-((4-(4-fluorophenyl)-2,5-dihydrothiophen-3-yl)methoxy)-3-methylphenyl)propanoic acid

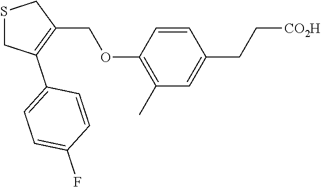 [SMILES: Cc1cc(CCC(=O)O)ccc1OCC1=C(c2ccc(F)cc2)CSC1]

The title compound was prepared as describe in General Synthesis Scheme 1, selecting and substituting suitable starting materials and reagents as would be readily recognized by those skilled in the art.

¹H NMR (CHLOROFORM-d) δ: 7.15-7.24 (m, 2H), 7.03-7.11 (m, 2H), 6.96-7.01 (m, 1H), 6.85-6.94 (m, 1H), 6.47-6.59 (m, 1H), 4.51 (s, 2H), 4.12 (br s, 4H), 2.77-2.90 (m, 3H), 2.57-2.69 (m, 3H), 2.20 (s, 3H).

### Example 114

### 3-(3,5-difluoro-4-((4-(4-fluorophenyl)-2,5-dihydrothiophen-3-yl)methoxy)phenyl)propanoic acid

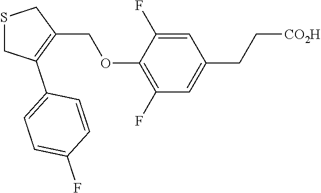 [SMILES: O=C(O)CCc1cc(F)c(OCC2=C(c3ccc(F)cc3)CSC2)c(F)c1]

The title compound was prepared as describe in General Synthesis Scheme 2, selecting and substituting suitable starting materials and reagents as would be readily recognized by those skilled in the art.

¹H NMR (CHLOROFORM-d) δ: 7.14-7.22 (m, 2H), 6.98-7.08 (m, 2H), 6.61-6.81 (m, 2H), 4.56 (s, 2H), 4.01-4.26 (m, 4H), 2.88 (s, 3H), 2.65 (s, 3H).

### Example 115

### 3-(4-((4-(4-fluorophenyl)-1-oxido-2,5-dihydrothiophen-3-yl)methoxy)-3-methylphenyl)propanoic acid

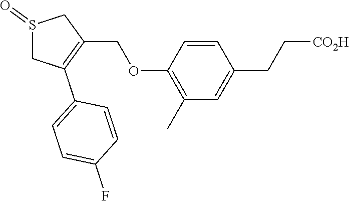 [SMILES: Cc1cc(CCC(=O)O)ccc1OCC1=C(c2ccc(F)cc2)CS(=O)C1]

The title compound was prepared as describe in General Synthesis Scheme 2, selecting and substituting suitable starting materials and reagents as would be readily recognized by those skilled in the art.

¹H NMR (CHLOROFORM-d) δ: 7.26 (s, 2H), 7.09 (s, 2H), 6.95-7.02 (m, 1H), 6.86-6.95 (m, 1H), 6.48-6.58 (m, 1H), 4.56-4.73 (m, 2H), 4.19-4.36 (m, 2H), 3.87-4.01 (m, 2H), 2.84 (s, 2H), 2.60 (s, 2H), 2.19 (s, 3H).

### Example #116

### 3-(4-((4-(4-fluorophenyl)-1,1-dioxido-2,5-dihydrothiophen-3-yl)methoxy)-3-methylphenyl)propanoic acid

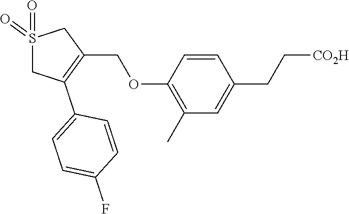 [SMILES: Cc1cc(CCC(=O)O)ccc1OCC1=C(c2ccc(F)cc2)CS(=O)(=O)C1]

The title compound was prepared as describe in General Synthesis Scheme 1, selecting and substituting suitable starting materials and reagents as would be readily recognized by those skilled in the art.

¹H NMR (CHLOROFORM-d) δ: 7.18-7.25 (m, 2H), 7.08-7.17 (m, 2H), 6.97-7.03 (m, 1H), 6.87-6.95 (m, 1H), 6.47-6.57 (m, 1H), 4.62 (s, 2H), 4.18 (d, J=8.1 Hz, 4H), 2.77-2.93 (m, 2H), 2.56-2.68 (m, 2H), 2.19 (s, 3H).

### Example #117

### 3-(3,5-difluoro-4-((4-(4-fluorophenyl)-1,1-dioxido-2,5-dihydrothiophen-3-yl)methoxy)phenyl)propanoic acid

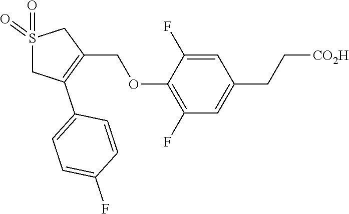 [SMILES: O=C(O)CCc1cc(F)c(OCC2=C(c3ccc(F)cc3)CS(=O)(=O)C2)c(F)c1]

The title compound was prepared as describe in General Synthesis Scheme 1, selecting and substituting suitable starting materials and reagents as would be readily recognized by those skilled in the art.

¹H NMR (CHLOROFORM-d) δ: 7.13-7.23 (m, 2H), 7.10 (d, J=8.6 Hz, 2H), 6.76 (d, J=8.6 Hz, 2H), 4.66 (s, 2H), 4.24 (s, 2H), 4.16 (s, 2H), 2.80-2.99 (m, 2H), 2.66 (s, 2H).

### Biological Example 1: In Vitro Assay

### Human GPR120 DiscoveRx PathHunter® Beta-Arrestin Assay

### Assay Principle:

The binding of an agonist (medium/long chain fatty acids or small molecule agonists) to the G-protein-coupled receptor GPR120 activates phospholipase C, leading to release of intracellular Ca⁺² through the generation of inositol 1,4,5-trisphosphate (InsP3 or IP3). GPR120 activation can also trigger intracellular signaling via recruitment of Beta-Arrestin. In the present method, agonist-induced activation of the human GPR120 receptor is monitored through the use of PathHunter® CHO-K1 GPR120 Beta-Arrestin Cell Line engineered by DiscoveRx, as detailed below. The cell lines were designed to co-express both the ProLink/Enzyme Donor (PK)-tagged GPCR and the Enzyme Activator (EA)-tagged Beta-Arrestin fusion proteins. Upon GPR120 receptor stimulation/activation, the EA-tagged Beta-Arrestin portion is translocated to the tagged receptor, where the two enzyme fragments are brought within close proximity. Under these conditions, these fragments can interact and form an active Beta-gal enzyme complex through Enzyme Fragment Complementation (EFC). This active Beta-gal complex can enzymatically hydrolyse the substrate to produce a detectable light signal; therefore, activation as a function of agonist concentration can be expressed as an EC₅₀ value to determine relative compound activities. This in vitro assay therefore serves to assess compound agonist activity of the GPR120.

### Procedure β-Arrestin A:

In Procedure β-arrestin A, the cell used were PathHunter® CHO-K1 GPR120 β-Arrestin Cell Line, expressing the long form of human GPR120 (Genbank accession number NM_181745), with 3000 cells per well.

### Procedure β-Arrestin B:

In Procedure β-arrestin B the cells used were PathHunter® CHO-K1 GPR120S β-Arrestin Cell Line, expressing the short form of the GPR120 receptor (Accession #NM_181745), with 5000 cells/well.

### Assay Procedure:

The selected CHO-K1 GPR120 β-Arrestin cells were cultured in Ham's F12 media supplemented with 10% fetal bovine serum (FBS), 1% Glutamine, 1× p/s, 800 μg/mL G418 and 300 μg/mL Hygromycin B (for selection). Cell stocks were maintained and grown in a sub-confluent state using standard cell culture procedures. The day before the experiment, the cells were harvested with non-enzymatic cell dissociation buffer and re-suspended in complete growth media at the desired concentration. A Corning 384-plate was then seeded with the proper number of cells in a volume of 25 μL, per well. The seeded plates were incubated overnight at 37° C.

On the day of the experiment, the Assay Buffer containing (a) HBSS with Ca⁺⁺ and Mg⁺⁺, (b) 20 mM HEPES, and (c) 0.1% BSA stabilizer (pH 7.4) was prepared. The growth medium was gently removed from the cell plates and 20 μL of Assay Buffer added to each well. The plate was then incubated at 37° C. for 60 min. Test compounds were serially diluted in Assay Buffer to desired concentrations (more particularly to one or more of the following μM concentrations: 25, 12.5, 6.25, 3.12, 1.56, 0.78, 0.39, 0.19, 0.10, 0.05, 0.02, 0.01). Five μL of compound dilution was then added to each well and the plate incubated at 37° C. for 90 min. The detection reagents were prepared according to the manufacture's instruction. Twelve μL of the detection reagents were added to each well and the plate incubated at room temperature for 60 min.

The plates were read on an EnVision instrument, using Protocol name: Luminescence, Plate type: 384 Costar, Measurement height: 3 mm, Measurement time: 1 s, Aperture: 384 Plate aperture. The % Activity relative to the positive control was calculated using the following equation:

$$\% Activity = \frac{{Count}_{compound} - {Count}_{vehicle}}{{Count}_{positive control} - {Count}_{vehicle}} \times 100 \%$$

The % Activity values were plotted versus the concentration of test compound and fitted to a sigmoidal dose-response curve with a Hill slope=1 (fixed value) using nonlinear regression with GraphPad Prism 5.0 to calculate the EC₅₀ values. The Fitting Equation was: Y=Bottom+(Top−Bottom)/(1+10\hat{ }((Log EC₅₀−X)*HillSlope)), where X is the log of the concentration and Y is the response.

### Biological Example 2: In Vitro Assay

### In Vitro Assay: Human GPR120 in Calcium Flux Assay

### Assay Principle

This in vitro assay serves to assess test compound agonist activity against the short splice variant (SVS with Accession number NM_001195755.1 confirmed by sequencing data) of the GPR120 receptor. The Human Short splice variant #2 (NM_001195755.1) is missing an in-frame coding exon compared to variant 1 (the Human Long splice variant NM_181745.3), resulting in a shorter isoform (GPR120-S) lacking a 16 aa protein segment compared to isoform GPR120-L. The assay platform utilizes HEK-293 cells stably transfected to express the Human GPR120 short form. These cells are first loaded with the Ca⁺² sensitive dye, Fluo-4 NW. Upon stimulation, intracellular released Ca⁺² can bind to the dye and alter its fluorescence intensity. This increase in fluorescence signal, and thus the flux in intracellular [Ca²⁺], is detected and quantitated by fluorescence imaging using a FLIPR reader. The effect of the agonist is measured as a function of concentration and used to calculate an EC₅₀ based upon a response curve.

### Procedure Calcium A:

In this procedure 2500 cells/well were employed.

### Procedure Calcium B:

In this procedure 4200 cells/well were employed.

### Assay Procedure:

A Human GPR120 clone (Genbank accession number NM_001195755.1) was placed into the pcDNA3.1 mammalian expression vector carrying the neomycin resistance gene. A stable mammalian cell was generated by placing the above clone into a HEK293 background. Clonal cells responding to long chain fatty acids had expression levels of GPR120 confirmed by RT-qPCR. Human HEK-GPR120 cells were cultured in Dulbecco's Modified Eagle's Medium (DMEM)/F12 medium supplemented with 10% fetal bovine serum (FBS), 1% L-Glutamine and 1% penicillin/streptomycin and 0.5 mg/ml G-418. Cells were split 2 times a week to keep the cells in the log-phase growth.

In preparation for the assay, HEK cells stably transfected with Human GPR120 (2.5K cells per well in 25 uL growth medium) were seeded into 384-well plates and then incubated overnight (37° C., 5% CO₂). The next day, the media was changed to 20 μL assay buffer and the cell starved for 1 h at 37° C. The dye loading solution (2× dye) was prepared using 10 mL assay buffer, 100 μL of 250 mM probenecid, 1 bottle of Component A, and 20 μl of dye in DMSO. Twenty μL of the 2× dye loading buffer was then added to each well. The plates were incubated at 37° C. for 30 min, then at room temperature for an additional 15 minutes, before performing the assay on FLIPR.

Test compounds were prepared in assay buffer (2 μL of compound+198 μL assay buffer, final DMSO in assay plate is 0.2%) at the desired concentration, more particularly at 100, 50, 25, 12.5, 6.25, 3.125, 1.562, 0.781, 0.391, 0.195, 0.098, 0.049, 0.024 and 0.012 μM.

The assay was performed on a FLIPR plate reader using the following parameters. Baseline was read for 10 seconds at 1 sec intervals. The program was set to transfer 10 μL of ligand from compound plate to cell plate after baseline reading. Aspiration was executed at: 10 μL/sec speed, 4.6 μL height; Dispensing was executed at: 30 μL/sec speed, 45 μL height. After compound addition, each well was read for 300 sec, with measurements collected at 1 sec intervals.

The kinetic data from the FLIPR was based upon a 5 minute window for data collection. The fluorescence of each sample well was used for individual calculations of a normalized RFU value, which was defined as maximum response minus the minimum response. The normalized fluorescence reading (RFU) was calculated as follows:

RFU=F max−F min

The data were fitted to a sigmoidal dose-response curve with a variable Hill slope (<2) using nonlinear regression with GraphPad Prism 5.0 to calculate the EC₅₀ values. The Fitting Equation was: Y=Bottom+(Top−Bottom)/(1+10\hat{ }((Log EC₅₀−X)*HillSlope)), where X is the log of the concentration and Y is the response.

### Biological Example 3: In Vitro Assay

### GPR40 Calcium Flux Assay

Compounds were tested in a calcium flux assay using transfected HEK293 cells stably expressing either human GPR40 or rat GPR40. Human GPR40 expressing cells were cultured in DMEM-High Glucose media supplemented with 10% fetal bovine serum, 1×L-Glutamine, 1× Penicillin/Streptomycin and 500 μg/mL G418. Rat GPR40 expressing cells were cultured in DMEM-High Glucose media supplemented with 10% fetal bovine serum and 1 μg/mL puromycin. Cells were plated into poly-D-lysine coated 384-well plates and cultured overnight in a 37° C. humidified tissue culture incubator under 5% CO₂/90% O₂ atmosphere.

On the day of the experiment, the culture media was replaced with assay buffer (HBSS, 20 mM HEPES, 0.1% BSA) and the cells incubated at 37° C. for 1 h. Calcium-sensitive fluorescent dye (Fluo 8 No-Wash Calcium Dye, ABD Bioquest) was then added and the cells incubated for another 30 min at 37° C. followed by 15 min at room temperature while protected from the light. The cell plate and a plate of diluted compounds of Formula (I) were loaded into a fluorescent plate reader that added compounds onto the cells while measuring the fluorescence intensity of each well. The plate reader recorded fluorescence intensity at 1 second intervals for 8 min and provided the data for analysis in an Excel format. EC₅₀ values were calculated using Prism (GraphPad) software.

Representative compounds of the present invention were tested according to the procedure as described in Biological Examples 1, 2 and 3, with results as listed in Table 5, below.

TABLE 5
Biological Assays: GPR40 and GPR120

| | GPR40 | Human GPR120 | | Human GPR120 | |
|---|---|---|---|---|---|
| | Calcium | Calcium Flux Assay | | Beta-Arrestin Assay | |
| | Flux | Procedure | Procedure | Procedure | Procedure |
| | Assay | A | B | A | B |
| ID No. | EC₅₀ (μM) | EC₅₀ (μM) | EC₅₀ (μM) | EC₅₀ (μM) | EC₅₀ (μM) |
| 620 | | 1.042 | 0.226 | 0.446 | |
| 621 | | 0.387 | 0.092 | 0.101 | |
| 625 | | | 0.124 | | 0.11 |
| 626 | | | 0.309 | | 0.317 |
| 627 | | | >5 | | |
| 628 | | | >5 | | |
| 629 | | | 3.984 | | |
| 630 | | | 0.229 | | |
| 631 | 0.364 | | 1.447 | | |
| 632 | 2.584 | | 0.422 | | |
| 633 | 1.127 | | 0.709 | | |
| 634 | 1.235 | | 0.503 | | |
| 640 | | | 0.538 | | 0.729 |
| 641 | | | 0.172 | | 0.204 |
| 642 | | | 0.176 | | 0.623 |
| 645 | | | 0.269 | | 0.275 |
| 646 | | | 0.628 | | 3.648 |
| 647 | | | >5 | | 0.74 |
| 650 | | | 0.316 | | 0.658 |
| 651 | | | 0.202 | | |
| 654 | | | >5 | | |
| 655 | | | 0.094 | | |
| 656 | | | 0.868 | | |
| 658 | | | 0.142 | | 0.169 |
| 659 | >10 | | 0.066 | | |
| 660 | | | 0.149 | | |
| 661 | | | 0.527 | | |
| 662 | | | 2.196 | | |
| 663 | | | 0.17 | | |
| 664 | | | >5 | | |
| 665 | | | 0.832 | | |
| 666 | | | 2.746 | | |
| 667 | | | 0.115 | | |
| 668 | | | 0.498 | | |
| 669 | | | >5 | | |
| 670 | | | >5 | | |
| 671 | | | >5 | | |
| 673 | | | >5 | | |
| 674 | | | >5 | | |
| 676 | | | >5 | | |
| 677 | | | >5 | | |
| 678 | | | >5 | | |
| 679 | | | 1.916 | | |
| 680 | | | 2.629 | | |
| 681 | | | >5 | | |
| 682 | | | >5 | | |
| 683 | | | 1.058 | | |
| 684 | 2.702 | | 0.786 | | |
| 685 | 0.829 | | 0.97 | | |
| 686 | 0.74 | | >5 | | |
| 687 | 0.054 | | >5 | | |
| 688 | 3.247 | | 2.936 | | |
| 691 | 3.624 | | 2.4 | | |
| 692 | 0.324 | | >5 | | |
| 695 | 3.187 | | >5 | | |
| 696 | 0.218 | | >5 | | |
| 697 | ~18.90 | | >5 | | |
| 698 | 0.335 | | >5 | | |
| 699 | 1.739 | | >5 | | |
| 701 | 0.145 | | >5 | | |
| 702 | 1.562 | | >5 | | |
| 703 | 0.068 | | >5 | | |
| 705 | 0.073 | | >5 | | |
| 706 | 0.038 | | >5 | | |
| 707 | 0.326 | | 0.829 | | |
| 709 | 2.297 | | >5 | | |
| 713 | 0.703 | | >5 | | |
| 714 | >10 | | 1.926 | | |
| 716 | 0.571 | | 1.836 | | |
| 717 | 0.133 | | 1.304 | | |
| 718 | 1.426 | | >5 | | |
| 719 | 1.018 | | >5 | | |
| 720 | 0.339 | | >5 | | |
| 721 | >10 | | 2.416 | | |
| 722 | 0.973 | | 1.191 | | |
| 723 | >10 | | >5 | | |
| 724 | >10 | | 0.275 | | |
| 725 | 0.588 | | 2.336 | | |
| 727 | 0.386 | | 1.598 | | |
| 729 | 0.847 | | 2.683 | | |
| 731 | 0.093 | | 0.818 | | |
| 732 | 1.064 | | >5 | | |
| 733 | >10 | | 1.807 | | |
| 734 | 0.744 | | 10.13 | | |
| 735 | 0.492 | | 2.187 | | |
| 736 | >10 | | 0.615 | | |
| 737 | 0.272 | | 0.515 | | |
| 738 | 2.68 | | 0.927 | | |
| 739 | 0.15 | | 1.02 | | |
| 740 | 2.078 | | 1.139 | | |
| 745 | >10 | | 0.11 | | 0.243 |
| 752 | | | 0.193 | | 0.159 |
| 753 | | | 0.178 | | 0.296 |
| 754 | | | 0.599 | | 0.894 |
| 755 | | | 0.778 | | |
| 756 | | | 0.429 | | |
| 757 | | | >5 | | |
| 758 | | | >5 | | |
| 759 | | | 1.121 | | |
| 760 | | | >5 | | |
| 761 | | | 2.897 | | |
| 762 | | | >5 | | |
| 763 | | | 0.546 | | |
| 764 | 1.435 | | 0.455 | | |
| 765 | 0.07 | | 0.478 | | |
| 766 | >10 | | 0.155 | | |
| 767 | 4.324 | | 0.131 | | |
| 768 | >10 | | 0.16 | | |
| 769 | >10 | | 1.093 | | |
| 775 | >10 | | 0.094 | | |
| 776 | | | 0.684 | | |
| 777 | | | 0.621 | | |
| 778 | 3.666 | | 0.379 | | |
| 779 | 2.032 | | 0.948 | | |
| 780 | 2.659 | | 1.31 | | |
| 781 | 4.143 | | 0.67 | | |
| 783 | 7.881 | | 1.357 | | |
| 784 | 0.187 | | 0.452 | | |
| 785 | 2.594 | | 0.274 | | |

### Biological Examide 4: In Vivo Assay

### GPR120 DIO Mice OGTT Screening

### Assay Procedure

18-22 week old, C57Bl6 mice on a high fat diet (60% HFD) for 12-16 weeks (average body weight ˜37-41 g) were fasted for 6 hr, with removal of food occurring at 7 am on the morning of the study. The animals were sorted into treatment groups the day before the study by body weight. Animals outside the bounds of ˜30-50 g were left out of the study. The animals had been handled and shammed a total of 5-8 days (1-3 days immediately prior to the study). Glucose (in 1 mL syringes) was drawn up the morning of the study. Test compounds were kept spinning and were only drawn into 1 ml syringes prior to study commencement. Animals were bled via tail snip to determine basal glucose levels prior to dosing of treatments. An Ascensia BREEZE Blood Glucose Monitoring System by Bayer was used for determining glucose levels.

Animals were moved into the testing room at ˜9-11 am, to yield them time to acclimate. The bleeds and dosing started at approximately 1 pm in 30-second intervals per animal. All groups were dosed 30 minutes prior to glucose administration at a dose volume of 10 ml/kg (the dose volume was calculated separately for each individual animal). Test compounds were administered at one or more of the following dosages: 0.1 mg/kg, 0.3 mg/kg, 1 mg/kg, 3 mg/kg and 10 mg/kg.

Thirty minutes after the first dose (with test compound) animals were bled again for a second baseline, or T=0, and immediately dosed with glucose (20% solution; TEKNOVA, 250 ml sterile bottle w/catalogue number G0525) via a PO injection. The exact dose volume for glucose was also calculated separately for each individual animal.

Blood glucose was measured at 15, 30, 45, 60, and 90 minutes post-glucose administration via the snipped tail. If an animal reached a value of “HI”, the upper limit of the glucometer (600 mg/dl) was substituted as the blood glucose value and the study was analyzed as normal with no exclusions. If 50% or more of any treatment group reaches a “HI”, value at least once, the study was considered invalid and repeated. Glucose values were typed into an EXCEL spreadsheet where they were used to calculate glucose AUC and delta AUC post-compound and post-glucose. The glucose excursion curves and the different versions of the AUC's were graphed in GraphPad Prism 5.

### Statistical Methods:

Note: All statistics completed in this study were completed using the statistical software package GraphPad Prism 5. Standard procedures for analyzing data sets from screening GPR120 compounds in DIO mouse OGTT's were as listed here below. In addition to the statistics that were run using GraphPad Prism 5, Microsoft Excel was used to calculate the percent changes in AUC from vehicle groups as detailed below.

Change from −30 to 0 Baseline Glucose, Raw Glucose AUC −30 to 90 min, Delta Glucose AUC −30 to 90 min, Raw Glucose AUC 0 to 90 min, Delta Glucose AUC 0 to 90 min were analyzed using Column Statistics Analysis, with mean values used to calculate % change from the vehicle mean group, as well as mean, SEM and/or % change from vehicle, where appropriate; and using One-Way ANOVA w/ a Tukey Post-Test (Comparing All Pairs of Columns) with each treatment group examined to see if it was statistically significant compared to vehicle (*=P<0.05, *=P<0.01, **=P<0.001).

Compound #745 was tested according to the procedure as described above, at a dosage of 3 mg/kg. DIO lowering (−30 to 90) was calculated at −63%.

### Biological Example 5: In Vivo Assay (Prophetic Example)

### A: GPR120 C57bl6 Mouse IPGTT

Male, C57bl6J Mice are ordered in at 8 weeks of age from Jackson Labs. Individual mice weigh anywhere in the range of 25-30 grams on study day. The mice are fasted, with removal of food occurring at 7 am on the morning of the study. Animals are moved into the room at 10:00 am, to give them time to acclimate. Glucose (insulin syringes) is drawn up either the night before or the morning of the study. Glucose is dosed (IP) at 1.5 g/kg at 7.5 ml/kg (20% glucose straight TEKNOVA, 250 ml sterile bottle w/catalogue number G0525). Test compounds are kept spinning and are only drawn into the syringes prior to study commencement. Animals are bled via tail snip to determine basal glucose levels prior to dosing of treatments. An Ascensia BREEZE Blood Glucose Monitoring System by Bayer (using unique 10-test disks) is used for determining glucose levels. The bleeds start at approximately 12:45 pm and dosing starts, at 1-minute intervals, immediately after. All groups are dosed 30 minutes prior to glucose administration at a dose volume of 10 ml/kg (the dose volume was calculated separately for each individual animal). Thirty minutes after the first dose animals are bled again for a second baseline, or T=0, and immediately dosed with glucose via an i.p. injection. The exact dose volume for glucose is also calculated separately for each individual animal. Glucose measurements are taken at −30 min prior to compound dose, at t=0 (immediately prior to glucose dose), and at 15, 30, 45, 60, 90 min post glucose dose.

Glucose values are entered into an Excel sheet and graphed in GraphPad Prism. The following can be calculated from GraphPad Prism: Change from −30 to 0 Baseline Glucose, Raw Glucose AUC −30 to 90 min, Delta Glucose AUC −30 to 90 min, Raw Glucose AUC 0 to 90 min, Delta Glucose AUC 0 to 90 min.

### Formulation Example 1 (Prophetic Example)

### Solid, Oral Dosage Form

As a specific embodiment of an oral composition, 100 mg of the Compound #745, prepared as described in Example 13 is formulated with sufficient finely divided lactose to provide a total amount of 580 to 590 mg to fill a size O hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

Throughout this application, various publications are cited. The disclosure of these publications is hereby incorporated by reference into this application to describe more fully the state of the art to which this invention pertains.

What is claimed:

1. A compound of formula (I) 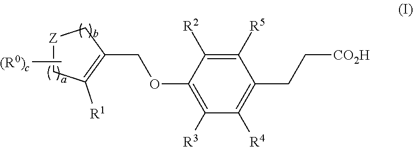
wherein
a is an integer from 1 to 2;
b is an integer from 1 to 2;
Z is selected from the group consisting of —O—, —S— and —SO₂—;
provided that when a is 1 and b is 2, or when a is 2 and b is 1 or when a is 2 and b is 2, then Z is —O—;
c is an integer from 0 to 2;
each R⁰ is independently selected from the group consisting of halogen, oxo, hydroxy, cyano, C₁₋₄alkyl, fluorinated C₁₋₂alkyl, C₁₋₄alkoxy and fluorinated C₁₋₂alkoxy;
R¹ is selected from the group consisting of C₁₋₆alkyl, C₂₋₆alkynyl, C₃₋₆cycloalkyl, —(C₁₋₂alkyl)-C₃₋₆cycloalkyl, C₅₋₆cycloalkenyl, bicyclo[3.1.0]hexy-2-yl, (1S,4S)-2-methyl-bicyclo[2.2.1]hept-2-yl, (1R,2R,4S)-bicyclo[2.2.1]heptan-2-yl, 2,2-difluoro-benzo[d][1,3]dioxol-4-yl, phenyl, —(C₁₋₂alkyl)-phenyl, —C(═CH)-phenyl, —C(O)-phenyl, tetrahydropyranyl, furanyl, pyrimidinyl, pyridyl, thienyl, thiazolyl, —(C₁₋₂alkyl)-thiazolyl, 3,6-dihydro-pyran-4-yl and 1-methyl-imidazol-4-yl;
wherein the C₃₋₆cycloalkyl or C₅₋₆cycloalkenyl, whether alone or as part of a substituent group is optionally substituted with one or more (preferably one to two) substituents independently selected from the group consisting of halogen, C₁₋₄alkyl and fluorinated C₁₋₂alkyl;
and wherein the phenyl, furanyl, pyrimidinyl, pyridyl, thienyl, thiazolyl, or 3,6-dihydro-pyran-4-yl, whether alone or as part of a substituent group is optionally substituted with one or more (preferably one to three) substituents independently selected from the group consisting of halogen, hydroxy, cyano, C₁₋₄alkyl, fluorinated C₁₋₂alkyl, C₁₋₄alkoxy, fluorinated C₁₋₂alkoxy, —S—(C₁₋₂alkyl), —SO—(C₁₋₂alkyl), —SO₂—(C₁₋₂alkyl), nitro, —NR^{A}R^{B}, —NH—C(O)—(C₁₋₄alkyl) and phenyl; wherein R^{A} and R^{B} are each independently selected from the group consisting of hydrogen and C₁₋₄alkyl;
R² is selected from the group consisting of hydrogen, halogen, C₁₋₄alkyl, fluorinated C₁₋₂alkyl, —(C₁₋₄alkyl)-S—(C₁₋₂alkyl), —(C₁₋₄alkyl)-SO—(C₁₋₂alkyl) and —(C₁₋₄alkyl)-SO₂—(C₁₋₂alkyl);
R³ is selected from the group consisting of hydrogen, halogen, C₁₋₄alkyl and fluorinated C₁₋₂alkyl;
R⁴ is selected from the group consisting of hydrogen, halogen, C₁₋₄alkyl and fluorinated C₁₋₂alkyl;
R⁵ is selected from the group consisting of hydrogen, cyano, halogen, C₁₋₄alkyl, fluorinated C₁₋₂alkyl, vinyl, halogen substituted vinyl, ethynyl, hydroxy substituted C₁₋₂alkyl, C₁₋₄alkoxy, fluorinated C₁₋₂alkoxy and cyclopropyl-methyl-;
alternatively, R² and R⁵ or R³ and R⁴ are taken together with the carbon atoms to which they are bound to form cyclopenten-1-yl;
or a pharmaceutically acceptable salt thereof.

2. A compound as in claim 1, wherein
a is an integer from 1 to 2;
b is an integer from 1 to 2;
Z is selected from the group consisting of —O—, —S—, —SO— and —SO₂—; provided that when a is 1 and b is 2, or when a is 2 and b is 1 or when a is 2 and b is 2, then Z is —O—;
c is an integer from 0 to 2;
each R⁰ is independently selected from the group consisting of halogen, oxo, hydroxy, C₁₋₄alkyl, fluorinated C₁₋₂alkyl, C₁₋₂alkoxy and fluorinated C₁₋₂alkoxy;
R¹ is selected from the group consisting of C₁₋₆alkyl, C₃₋₆cycloalkyl, —(C₁₋₂alkyl)-C₃₋₆cycloalkyl, C₅₋₆cycloalkenyl, 2,2-difluoro-benzo[d][1,3]dioxol-4-yl, phenyl, —(C₁₋₂alkyl)-phenyl, —C(═CH)-phenyl, —C(O)-phenyl, pyrimidinyl, pyridyl, thienyl, and 1-methyl-imidazol-4-yl;
wherein the C₃₋₆cycloalkyl or C₅₋₆cycloalkenyl, whether alone or as part of a substituent group is optionally substituted with one to two substituents independently selected from the group consisting of halogen, C₁₋₂alkyl and fluorinated C₁₋₂alkyl;
and wherein the phenyl, pyrimidinyl or pyridyl, whether alone or as part of a substituent group is optionally substituted with one to three substituents independently selected from the group consisting of halogen, hydroxy, C₁₋₄alkyl, fluorinated C₁₋₂alkyl, C₁₋₄alkoxy, fluorinated C₁₋₂alkoxy, and phenyl;
R² is selected from the group consisting of hydrogen, halogen, C₁₋₄alkyl and fluorinated C₁₋₂alkyl;
R³ is selected from the group consisting of hydrogen, halogen, C₁₋₂alkyl and fluorinated C₁₋₂alkyl;
R⁴ is selected from the group consisting of hydrogen, halogen, C₁₋₄alkyl and fluorinated C₁₋₂alkyl;
R⁵ is selected from the group consisting of hydrogen, halogen, C₁₋₄alkyl, fluorinated C₁₋₂alkyl, hydroxy substituted C₁₋₂alkyl, C₁₋₄alkoxy, fluorinated C₁₋₂alkoxy and cyclopropyl-methyl-;
alternatively, R² and R⁵ or R³ and R⁴ are taken together with the carbon atoms to which they are bound to form cyclopenten-1-yl;
or a pharmaceutically acceptable salt thereof.

3. A compound as in claim 2, wherein
a is an integer from 1 to 2;
b is an integer from 1 to 2;
Z is selected from the group consisting of —O—, —S—, —SO— and —SO₂—; provided that when a is 1 and b is 2, or when a is 2 and b is 1 or when a is 2 and b is 2, then Z is —O—;
c is an integer from 0 to 2;
each R⁰ is independently selected from the group consisting of C₁₋₄alkyl;
R¹ is selected from the group consisting of C₁₋₆alkyl, —C(═CH)-phenyl, C₃₋₆cycloalkyl, C₅₋₆cycloalkenyl, —(C₁₋₂alkyl)-C₅₋₆cycloalkyl, phenyl, —(C₁₋₂alkyl)-phenyl, pyrid-3-yl, and 2,2-difluoro-benzo[d][1,3]dioxol-4-yl;
wherein the C₅₋₆cycloalkylenyl, is optionally substituted with one to two C₁₋₂alkyl;
wherein the phenyl, whether alone or as part of a substituent group, is optionally substituted with one to three substituents independently selected from the group consisting of halogen, C₁₋₄alkyl, fluorinated C₁₋₂alkyl, C₁₋₄alkoxy, fluorinated C₁₋₂alkoxy and phenyl;
R² is selected from the group consisting of hydrogen, halogen and C₁₋₄alkyl;
R³ is selected from the group consisting of hydrogen and halogen;
R⁴ is selected from the group consisting of hydrogen, C₁₋₄alkyl and fluorinated C₁₋₂alkyl;
R⁵ is selected from the group consisting of hydrogen, halogen, C₁₋₄alkyl, fluorinated C₁₋₂alkyl and cyclopropyl-methyl-;
or a pharmaceutically acceptable salt thereof.

4. A compound as in claim 3, wherein
a is an integer from 1 to 2;
b is an integer from 1 to 2;
Z is selected from the group consisting of —O—, —S—, —SO— and —SO₂—; provided that when a is 1 and b is 2, or when a is 2 and b is 1 or when a is 2 and b is 2, then Z is —O—;
c is an integer from 0 to 2;
each R⁰ is methyl;
R¹ is selected from the group consisting of ethyl, isopropyl, n-butyl, 1-methyl-n-propyl, isobutyl, isopentyl, n-hexyl, cyclobutyl, cyclopentyl, cyclohexyl, cyclopent-2-en-1-yl, 5,5-dimethyl-cyclopenten-1-yl, cyclohexyl-methyl-, phenyl, 2-fluorophenyl, 4-fluorophennyl, 4-chlorophenyl, 4-methylphenyl, 2-methoxyphenyl, 3-methoxyphenyl, 4-methoxyphenyl, 2-isopropyloxyphenyl, 2-trifluoromethoxy-phenyl, 4-trifluoromethoxy-phenyl, 2,4-difluorophenyl, 3,4-difluorophenyl, 3,4-dichlorophenyl, 2-fluoro-4-chloro-phenyl, 2-fluoro-4-methyl-phenyl, 3-fluoro-4-chloro-phenyl, 3-fluoro-4-methyl-phenyl, 2-methyl-4-chloro-phenyl, 3-methyl-4-fluoro-phenyl, 3-methyl-4-chloro-phenyl, 2-isopropyl-4-methyl-phenyl, 3-isopropyl-4-methyl-phenyl, 2-methoxy-4-chloro-phenyl, 3-methoxy-4-chloro-phenyl, 2,4-dimethyl-phenyl, 2,6-dimethyl-phenyl, 3,4-dimethyl-phenyl, 2,4,5-trimethyl-phenyl, 2-phenyl-4-methyl-phenyl, 3-phenyl-4-methyl-phenyl, benzyl, 4-chloro-benzyl, 4-methyl-benzyl, 4-methoxy-benzyl, 1-phenyl-ethyl-, 1-phenyl-vinyl-, 1-(4-fluorophenyl)-vinyl-, 1-(2-chloro-phenyl)-vinyl-, 1-(4-trifluoromethyl-phenyl)-vinyl-, pyrid-3-yl, and 2,2-difluoro-benzo[d][1,3]dioxol-4-yl;
R² is selected from the group consisting of hydrogen, fluoro and methyl;
R³ is selected from the group consisting of hydrogen and fluoro;
R⁴ is selected from the group consisting of hydrogen, methyl and trifluoromethyl;
R⁵ is selected from the group consisting of hydrogen, fluoro, chloro, methyl, difluoromethyl, trifluoromethyl and cyclopropyl-methyl-;
or a pharmaceutically acceptable salt thereof.

5. A compound as in claim 4, wherein
a is 1;
b is 1;
Z is selected from the group consisting of —O—, —S—, —SO— and —SO₂—;
c is 0;
R¹ is selected from the group consisting of phenyl, 4-chlorophenyl, 4-fluorophenyl and cyclopentyl;
R² is selected from the group consisting of hydrogen, fluoro and methyl;
R³ is selected from the group consisting of hydrogen and fluoro;
R⁴ is hydrogen;
R⁵ is selected from the group consisting of hydrogen, fluoro, methyl and trifluoromethyl;
or a pharmaceutically acceptable salt thereof.

6. A compound as in claim 4, wherein
a is 1;
b is 2;
Z is —O—;
c is an integer from 0 to 2;
each R⁰ is 6-methyl;
R¹ is selected from the group consisting of ethyl, isopropyl, n-butyl, 1-methyl-n-propyl, isobutyl, isopentyl, n-hexyl, 2-fluorophenyl, 4-fluorophennyl, 4-chlorophenyl, 4-methylphenyl, 2-methoxyphenyl, 3-methoxyphenyl, 4-methoxyphenyl, 2-isopropyloxyphenyl, 2-trifluoromethoxy-phenyl, 4-trifluoromethoxy-phenyl, 2,4-difluorophenyl, 3,4-difluorophenyl, 3,4-dichlforophenyl, 2-fluoro-4-chloro-phenyl, 2-fluoro-4-methyl-phenyl, 3-fluoro-4-chloro-phenyl, 3-fluoro-4-methyl-phenyl, 2-methyl-4-chloro-phenyl, 3-methyl-4-fluoro-phenyl, 3-methyl-4-chloro-phenyl, 2-methoxy-4-chloro-phenyl, 3-methoxy-4-chloro-phenyl, 2,6-dimethyl-phenyl, 3,4-dimethyl-phenyl, 2,4,5-trimethyl-phenyl, benzyl, 4-chloro-benzyl, 4-methyl-benzyl, 4-methoxy-benzyl, 1-phenyl-ethyl-, 1-phenyl-vinyl-, 1-(4-fluorophenyl)-vinyl-, 1-(2-chloro-phenyl)-vinyl-, 1-(4-trifluoromethyl-phenyl)-vinyl-, cyclobutyl, cyclopentyl, cyclohexyl, cyclopent-2-en-1-yl, 5,5-dimethyl-cyclopenten-1-yl, cyclohexyl-methyl-, pyrid-3-yl, and 2,2-difluoro-benzo[d][1,3]dioxol-4-yl;
R² is selected from the group consisting of hydrogen, fluoro and methyl;
R³ is selected from the group consisting of hydrogen and fluoro;
R⁴ is selected from the group consisting of hydrogen and trifluoromethyl;
R⁵ is selected from the group consisting of hydrogen, chloro, methyl, difluoromethyl, and trifluoromethyl;
or a pharmaceutically acceptable salt thereof.

7. A compound as in claim 4, wherein
a is 2;
b is 1;
Z is —O—;
c is 0;
R¹ is selected from the group consisting of phenyl, 4-chlorophenyl, 4-methylphenyl, 2-fluoro-4-methyl-phenyl, 3-fluoro-4-methyl-phenyl, 2,4-dimethyl-phenyl, 2-isopropyl-4-methyl-phenyl, 3-isopropyl-4-methyl-phenyl, 2-phenyl-4-methyl-phenyl, 3-phenyl-4-methyl-phenyl, and cyclopentyl;
R² is selected from the group consisting of hydrogen, fluoro and methyl;
R³ is selected from the group consisting of hydrogen and fluoro;
R⁴ is hydrogen;
R⁵ is selected from the group consisting of hydrogen, fluoro, methyl, trifluoromethyl, and cyclopropyl-methyl-;
or a pharmaceutically acceptable salt thereof.

8. A compound as in claim 4, wherein
a is 2;
b is 2;
Z is —O—;
c is 0;
R¹ is selected from the group consisting of 4-chlorophenyl, 4-methylphenyl, 3-fluoro-4-methyl-phenyl, and cyclopentyl;
R² is selected from the group consisting of hydrogen, fluoro and methyl;
R³ is selected from the group consisting of hydrogen and fluoro;
R⁴ is selected from the group consisting of hydrogen and methyl;
R⁵ is selected from the group consisting of hydrogen, fluoro, chloro, methyl and trifluoromethyl;
or a pharmaceutically acceptable salt thereof.

9. A compound as in claim 4, wherein
a is an integer from 1 to 2;
b is an integer from 1 to 2;
Z is —O—;
c is 0;
R¹ is selected from the group consisting of ethyl, isopropyl, 1-methyl-n-propyl, n-butyl, isobutyl, isopentyl, cyclobutyl, cyclopentyl, cyclohexyl, cyclopent-2-en-1-yl, 4-trifluoromethoxy-phenyl, 3-fluoro-4-methyl-phenyl, benzyl, 4-chloro-benzyl, 4-methyl-benzyl, 4-methoxy-benzyl, 1-phenyl-vinyl, 1-(2-chloro-phenyl)-vinyl, 1-(4-fluoro-phenyl)-vinyl and 1-(4-trifluoromethyl-phenyl)-vinyl;
R² is hydrogen;
R³ is hydrogen;
R⁴ is hydrogen;
R⁵ is selected from the group consisting of chloro and trifluoromethyl;
or a pharmaceutically acceptable salt thereof.

10. A compound as in claim 4, wherein
a is an integer from 1 to 2;
b is an integer from 1 to 2;
Z is —O—;
c is 0;
R¹ is selected from the group consisting of isopropyl, n-butyl, isobutyl, cyclopentyl, cyclopent-2-en-1-yl, 4-chloro-benzyl, 4-methyl-benzyl, 4-methoxy-benzyl, 1-phenyl-vinyl, 1-(2-chloro-phenyl)-vinyl, 1-(4-fluoro-phenyl)-vinyl and 1-(4-trifluoromethyl-phenyl)-vinyl;
R² is hydrogen;
R³ is hydrogen;
R⁴ is hydrogen;
R⁵ is selected from the group consisting of chloro and trifluoromethyl;
or a pharmaceutically acceptable salt thereof.

11. A compound as in claim 4, wherein
a is an integer from 1 to 2 and b is 2;
alternatively a is 2 and b is an integer from 1 to 2;
Z is —O—;
c is 0;
R¹ is selected from the group consisting of isobutyl, cyclopentyl, cyclopent-2-en-1-yl, 4-chloro-benzyl, 1-phenyl-vinyl, 1-(2-chloro-phenyl)-vinyl, 1-(4-fluoro-phenyl)-vinyl and 1-(4-trifluoromethyl-phenyl)-vinyl;
R² is hydrogen;
R³ is hydrogen;
R⁴ is hydrogen;
R⁵ is trifluoromethyl;
or a pharmaceutically acceptable salt thereof.

12. A compound as in claim 4, wherein
a is 1 and b is 2;
alternatively a is 2 and b is 1;
Z is —O—;
c is 0;
R¹ is selected from the group consisting of cyclopentyl, 1-phenyl-vinyl, 1-(2-chloro-phenyl)-vinyl, 1-(4-fluoro-phenyl)-vinyl and 1-(4-trifluoromethyl-phenyl)-vinyl;
R² is hydrogen;
R³ is hydrogen;
R⁴ is hydrogen;
R⁵ is trifluoromethyl;
or a pharmaceutically acceptable salt thereof.

13. A compound as in claim 4, wherein
a is an integer from 1 to 2;
b is an integer from 1 to 2;
Z is selected from the group consisting of —O— and —S—; provided that when a is 1 and b is 2, or when a is 2 and b is 1 or when a is 2 and b is 2, then Z is —O—;
c is 0;
R¹ is selected from the group consisting of cyclobutyl, cyclopentyl, phenyl, 4-chlorophenyl, 2-fluorophenyl, 4-fluorophenyl, 4-methylphenyl, 2,4-difluorophenyl, 2-fluoro-4-chloro-phenyl, 3,4-difluorophenyl, 3-fluoro-4-chloro-phenyl and 3-fluoro-4-methyl-phenyl;
R² is selected from the group consisting of hydrogen, fluoro and methyl;
R³ is selected from the group consisting of hydrogen and fluoro;
R⁴ is hydrogen;
R⁵ is selected from the group consisting of hydrogen, fluoro, methyl and trifluoromethyl;
or a pharmaceutically acceptable salt thereof.

14. A compound as in claim 4, wherein
a is an integer from 1 to 2;
b is an integer from 1 to 2;
Z is selected from the group consisting of —O— and —S—; provided that when a is 1 and b is 2, or when a is 2 and b is 1 or when a is 2 and b is 2, then Z is —O—;
c is 0;
R¹ is selected from the group consisting of phenyl, 4-chlorophenyl, 2-fluorophenyl, 4-fluorophenyl, 4-methylphenyl, 2,4-difluorophenyl, 2-fluoro-4-chloro-phenyl, 3,4-difluorophenyl, 3-fluoro-4-chloro-phenyl and 3-fluoro-4-methyl-phenyl;
R² is selected from the group consisting of fluoro and methyl;
R³ is selected from the group consisting of hydrogen and fluoro;
R⁴ is hydrogen;
R⁵ is selected from the group consisting of hydrogen, fluoro and methyl;
or a pharmaceutically acceptable salt thereof.

15. A compound as in claim 4, wherein
a is 1 and b is 1; alternatively, a is 2 and b is 1; alternatively, a is 2 and b is 2;
Z is —O—;
c is 0;
R¹ is selected from the group consisting of 4-chlorophenyl, 4-methylphenyl and 3-fluoro-4-methyl-phenyl;
R² is methyl;
R³ is hydrogen;
R⁴ is hydrogen;
R⁵ is methyl;
or a pharmaceutically acceptable salt thereof.

16. A compound as in claim 4, selected from the group consisting of
3-[4-[[5-(4-chlorophenyl)-2,3,6,7-tetrahydrooxepin-4-yl]methoxy]-2,3-dimethyl-phenyl]propanoic acid;
3-[4-[[5-(p-tolyl)-2,3,6,7-tetrahydrooxepin-4-yl]methoxy]-2-(trifluoromethyl)phenyl]propanoic acid;
3-[4-[(5-cyclopentyl-2,3,6,7-tetrahydrooxepin-4-yl)methoxy]-2-(trifluoromethyl)phenyl]propanoic acid;
3-[4-[[5-(4-chlorophenyl)-3,6-dihydro-2H-pyran-4-yl]methoxy]-2,3-dimethyl-phenyl]propanoic acid;
3-[4-[[5-(4-chlorophenyl)-3,6-dihydro-2H-pyran-4-yl]methoxy]-2-(trifluoromethyl)phenyl]propanoic acid;
3-[4-[[5-(p-tolyl)-3,6-dihydro-2H-pyran-4-yl]methoxy]-2-(trifluoromethyl)phenyl]propanoic acid;
3-[4-[(5-phenyl-3,6-dihydro-2H-pyran-4-yl)methoxy]-2-(trifluoromethyl)phenyl]propanoic acid;
3-[4-[(5-cyclopentyl-3,6-dihydro-2H-pyran-4-yl)methoxy]-2-(trifluoromethyl)phenyl]propanoic acid;
3-[4-[[5-(3-fluoro-4-methyl-phenyl)-3,6-dihydro-2H-pyran-4-yl]methoxy]-2,3-dimethyl-phenyl]propanoic acid;
3-[4-[[4-(4-chlorophenyl)-3,6-dihydro-2H-pyran-5-yl]methoxy]-2,3-dimethyl-phenyl]propanoic acid;
3-[4-[[4-(4-chlorophenyl)-3,6-dihydro-2H-pyran-5-yl]methoxy]-2-(trifluoromethyl)phenyl]propanoic acid;
3-[4-[(4-ethyl-3,6-dihydro-2H-pyran-5-yl)methoxy]-2-(trifluoromethyl)phenyl]propanoic acid;
3-[2-chloro-4-[(4-cyclopentyl-3,6-dihydro-2H-pyran-5-yl)methoxy]phenyl]propanoic acid;
3-[4-[(4-cyclopentyl-3,6-dihydro-2H-pyran-5-yl)methoxy]-2-(trifluoromethyl)phenyl]propanoic acid;
and pharmaceutically acceptable salts thereof.

17. A pharmaceutical composition comprising a pharmaceutically acceptable carrier and a compound of claim 1.

18-19. (canceled)

20. A method of treating a disorder which (a) is modulated by the GPR120 receptor, (b) is modulated by the GPR40 receptor, or (c) responds to dual agonism of the GPR120 and GPR40 receptors comprising administering to a subject in need thereof a therapeutically effective amount of the compound of claim 1.

21. The method of claim 20, wherein the disorder is selected from the group consisting of obesity, obesity induced inflammation, impaired glucose tolerance, elevated fasting glucose, insulin resistance, hyperglycemia, hyperinsulinemia, Type II Diabetes Mellitus, metabolic syndrome (also known as Syndrome X), gestational diabetes, diabetic retinopathy, kidney disease, ketoacidosis, diabetic nephropathy, dyslipidemia, elevated LDL, hyperipidemia, hyperlipoproteinemia, hypertriglyceridemia, non-alcoholic steatohepatitis (NASH), non-alcoholic fatty liver disease (NAFLD), liver fibrosis and cardiovascular disorders.

22. The method of claim 20, wherein the disorder is selected from the group consisting of obesity, impaired glucose tolerance, elevated fasting glucose, insulin resistance, hyperglycemia, hyperinsulinemia, Type II Diabetes Mellitus, metabolic syndrome (also known as Syndrome X), diabetic retinopathy, diabetic nephropathy, dyslipidemia, elevated LDL, hyperipidemia, hypertriglyceridemia, non-alcoholic steatohepatitis (NASH) and non-alcoholic fatty liver disease (NAFLD).

23. The method of claim 20, wherein the disorder is selected from the group consisting of obesity, Type II diabetes, metabolic syndrome (also known as Syndrome X), dyslipidemia. hypertriglyceridemia (i.e. elevated triglycerides), non-alcoholic steatohepatitis (NASH) and non-alcoholic fatty liver disease (NAFLD).

24. The method of claim 20, wherein the disorder is selected from the group consisting of obesity, Type II diabetes, metabolic syndrome (also known as Syndrome X), dyslipidemia and hypertriglyceridemia.

25. A method of treating a disorder selected from the group consisting of obesity, obesity induced inflammation, impaired glucose tolerance, elevated fasting glucose, insulin resistance, hyperglycemia, hyperinsulinemia, Type II Diabetes Mellitus, metabolic syndrome (also known as Syndrome X), gestational diabetes, diabetic retinopathy, kidney disease, ketoacidosis, diabetic nephropathy, dyslipidemia, elevated LDL, hyperipidemia, hyperlipoproteinemia, hypertriglyceridemia, non-alcoholic steatohepatitis (NASH), non-alcoholic fatty liver disease (NAFLD), liver fibrosis and cardiovascular disorders comprising administering to a subject in need thereof a therapeutically effective amount of a compounds as in claim 1.

26-39. (canceled)
